(19) United States
(12) Patent Application Publication
(10) Pub. No.: US 2019/0331682 A1
(43) Pub. Date: Oct. 31, 2019

(54) METHODS AND KITS FOR PREDICTING THE SENSITIVITY OF A SUBJECT TO IMMUNOTHERAPY

(71) Applicant: INSTITUT GUSTAVE ROUSSY, VILLEJUIF (FR)

(72) Inventors: LAURENCE ZITVOGEL, PARIS (FR); NICOLAS JACQUELOT, THIAIS (FR); DAVID ENOT, CREULLY (FR); SYLVIE SEIJO-RUSAKIEWICZ, EVIAN LES BAINS (FR)

(21) Appl. No.: 16/077,747

(22) PCT Filed: Feb. 17, 2017
(86) PCT No.: PCT/EP2017/053577

§ 371 Date: Aug. 14, 2018

(30) Foreign Application Priority Data
Feb. 18, 2016 (EP) 16305185.7
Apr. 15, 2016 (EP) 16305445.5

(51) Int. Cl. G01N 33/574

(52) U.S. Cl. CPC G01N 33/5743; G01N 2333/70514; G01N 2333/70517; G01N 2333/70532; G01N 2333/7155; G01N 2333/7156; G01N 2333/70596

(57) ABSTRACT

The present invention relates to a method of predicting assessing or monitoring the sensitivity of a subject having a cancer to an immunotherapy, and to corresponding kits. The method of predicting, assessing or monitoring the sensitivity of a subject having a tumor to an immunotherapy typically comprises a step a) of determining, in a biological sample from said subject, the presence, absence or expression level of at least one biomarker, for example at least two biomarkers, and when the expression level is determined a step b) of comparing said expression level to reference expression level(s) or to reference expression ratio(s), thereby predicting, assessing or monitoring whether the subject having a tumor is responsive or resistant to the proposed immunotherapy.

### CROSS-REFERENCE TO RELATED APPLICATION

This application is the U.S. national stage application of International Patent Application No. PCT/EP2017/053577, filed Feb. 17, 2017.

### FIELD OF THE INVENTION

The present invention relates to a method of predicting, assessing or monitoring the sensitivity of a subject having a cancer or malignant tumor to immunotherapy, and to corresponding kits. The method of predicting, assessing or monitoring the sensitivity of a subject having a cancer or malignant tumor to a proposed immunotherapy typically comprises a step a) of determining, in a biological sample from said subject, the presence, absence or expression level of at least one biomarker, for example at least two biomarkers, and when the expression level is determined a step b) of comparing said expression level to reference expression level(s) or to reference expression ratio(s), thereby predicting, assessing or monitoring whether the subject having a tumor is responsive or resistant to the proposed immunotherapy.

### BACKGROUND OF THE INVENTION

In the past decade, progress in tumor biology, genomics technology, computational analysis and drug discovery has propelled advances in translational and clinical cancer research. Inventors learned from genetic analyses of many cancers that specific cancer types often depend on deregulation of a limited set of signaling pathways, such as BRAF mutations in melanoma, c-KIT/PDGFRA tyrosine kinase activation in GIST, or Rb, KRAS, HER2 oncogene products in SCLC, pancreatic or breast cancers respectively. Despite the heterogeneity of different tumor subclones, the convergence of distinct aberrant signaling pathways lead to the general notion of “oncogene addiction” which prevails to support the current strategies of “personalized medicine approaches” (1). Lung cancer has become the prototype for genetically tailored targeted therapies (EGFR, KRAS, BRAF, CRAF, PI3KCA, PTEN, LKB1, RAC1, p53, etc.). However, only a small fraction of lung cancers are “oncogene-addicted” and may benefit from this molecular alteration-guided “personalized” medicine approach (2). The recent development of immunotherapeutic compounds rekindled the field of cancer immunotherapy (3, 4), bypassing the need for a driving mutation. Cancer vaccines (5) (Sipuleucel T), adoptive T cell transfer and CAR T cells (6, 7), bispecific antibodies (8), immune checkpoint blockers (9, 10) and oncolytic viruses (11) came of age and entered the oncological armamentarium.

However, to date immunotherapy has been shown to induce durable clinical benefit in only a fraction of patients. The recent characterization of multiple immune resistance mechanisms used by the tumor to evade the immune system fueled the development of novel agents that circumvent those limitations, targeting new “immune checkpoints”. Therefore, it is likely that the use of combination strategies will increase the number of patients that might benefit from immunotherapy (12).

Several critical issues remain open conundrums. First, the scientific rationale lending support to distinct combinatorial regimen needs to be outlined. Second, one has to determine whether the future of immuno-oncology (I-O) will rely on patients' stratification or will be personalized. Indeed, depending on tumor criteria (for instance PD-L1 or PD-1 expression on tumor cells for anti-PD-1Ab (13-15) or HMGB1 and LC3B for immunogenic immunotherapy (16) or tumor microenvironmental hallmarks [IDO expression (17), macrophages density (18), TIL or TH1 fingerprints (19)], one might be inclined to envisage more specific and individualized I-O clinical management. Thirdly, predictive immune profiles or biomarkers will have to be validated prospectively to guide I-O in a personalized or stratified manner.

Inventors herein address some of these questions in particular in melanoma, given that i) adjuvant efficient I-O remain an unmet medical need, ii) metastatic melanoma represent the clinical niche for the development of most if not all mAb and immune checkpoint blockers (ICB), iii) metastatic lymph nodes (mLN) are surgically resected, enabling for immunological investigations, iv) they already reported immune prognostic parameters in stage III/IV melanoma (Jacquelot N et al, JID in press, Jacquelot N et al, JCI in press).

The complexity of the regulation of the tumor microenvironment, and the various and independent mechanisms of action of all these checkpoint/costimulatory axes, call for comprehensive analyses of their mode of action in the tumor microenvironment of a given patient to design appropriate combinatorial approaches and discover specific biomarkers of response. Using a system biology approach aimed at defining immunometrics relevant for prediction of in situ response to classical therapeutic cytokines and novel mAb (agonistic and blockers of immune checkpoints) on freshly resected stage III melanoma, inventors herein describe in particular a suitable “ex vivo mLN assay” and unravel immune biomarkers of interest for a prospective clinical validation of the concept of personalized I-O.

### SUMMARY OF THE INVENTION

Personalized therapy of cancer currently relies on the identification of drug targetable tumor cell autonomous signaling pathways. However, immunomodulation of the tumor microenvironment may also be amenable to a more personalized management and predictive tools for this decision making are awaited.

Inventors herein identify predictive biomarkers that are able to secure identification of cancer patients proned to respond or resist to a proposed immunotherapy. The present invention includes methods and kits for predicting, assessing or monitoring the response of a subject having cancer (herein equivalent to “malignant tumor”) to a particular chemotherapeutic treatment using these biomarkers.

A first method herein described is an in vitro or ex vivo method of predicting, assessing or monitoring the sensitivity of a subject having a cancer to a proposed immunotherapy. The method typically comprises a step a) of determining, in a biological sample from said subject, the presence, absence or expression level of at least one biomarker, for example at least two biomarkers, and when the expression level is determined a step b) of comparing said expression level to reference expression level(s) or to reference expression ratio(s), thereby predicting, assessing or monitoring whether the subject having a tumor is responsive or resistant to the proposed immunotherapy.

A particular method herein described is an in vitro method of assessing the sensitivity of a subject having a cancer to an immunotherapy selected from anti-PD-1 monoclonal antibody, anti-PD-L1 monoclonal antibody, anti-CTLA-4 monoclonal antibody, anti-CD137 monoclonal antibody, anti-CD137L monoclonal antibody, anti-TIM3 monoclonal antibody, IFNα2a (ROF), IL-2, a combination of anti-PD-1 and anti-CTLA-4 monoclonal antibodies, a combination of anti-PD-1 monoclonal antibody and ROF, a combination of anti-CTLA-4 monoclonal antibody and ROF, and a combination of anti-PD-1 and anti-TIM3 monoclonal antibodies, in particular to an immunotherapy selected from anti-PD-1 monoclonal antibody, anti-PD-L1 monoclonal antibody, anti-CTLA-4 monoclonal antibody and a combination of anti-PD-1 and anti-CTLA-4 monoclonal antibodies, which method comprises a step a) of determining, in a biological sample from said subject which is a blood sample or a sample comprising tumor cells, the presence, absence or expression level of at least one biomarker, for example at least two biomarkers, selected from PD-1⁺CD4⁺ T cells, CD8+ T cells and CD25⁺CD127⁻CD4⁺ T cells, CD95⁺CD4⁺ T cells, CD95⁺CD8⁺ T cells, PD-L1⁺CD4⁺ T cells, PD-L1⁺CD8⁺ T cells, CLA⁺CD8⁺ TEM cells, CD137L⁺CD4⁺ T cells, CD137L⁺CD8⁺ T cells, CD137⁺CD4⁺ T cells and CD137⁺CD8⁺ T cells, and, when the expression level is determined, a step b) of comparing said at least one expression level to a reference expression level or to a reference expression ratio, thereby predicting, assessing or monitoring whether the subject having a cancer is responsive or resistant to the immunotherapy.

Also herein described is an assay for determining whether a patient is sensitive or resistant to a cancer therapy (also herein identified as ex vivo “mLN assay”), wherein the assay comprises:
a first step wherein suspensions of metastatic lymph nodes samples are incubated ex vivo in duplicate wells, each well of each set of the duplicate being in contact with medium, with a control antibody, or with a test immunotherapeutic antibody defining a cancer therapy, said antibody being preferably selected from an anti-PD-1 monoclonal antibody, an anti-PD-L1 monoclonal antibody, an anti-CTLA-4 monoclonal antibody, an anti-CD137 monoclonal antibody, an anti-CD137L monoclonal antibody, an anti-TIM3 monoclonal antibody, an IFNα2a (ROF), an IL-2, a combination of anti-PD-1 and anti-CTLA-4 monoclonal antibodies, a combination of anti-PD-1 monoclonal antibody and ROF, a combination of anti-CTLA-4 monoclonal antibody and ROF, or a combination of anti-PD-1 and anti-TIM3 monoclonal antibodies, the first set of wells being incubated for 18h-24h, and the second set of wells being incubated for 4 to 5 days,
a second step of measuring T cells, NK cells and/or Treg cells parameters, said parameters consisting in cell biomarker(s) expression, cytokine cell release, interferon cell release, chemokine cell release and/or interleukin cell release in the first set of wells, and Ki67 cell expression and Treg cell proportion in the second set of wells, and
a third step of comparing measures obtained in each well with the corresponding measure obtained from the medium and control wells, a 1.5 fold variation of at least two parameters indicating that the patient is sensitive to the cancer therapy.

Another particular method herein described is a method of selecting an appropriate chemotherapeutic treatment for a subject, which method comprises a step of predicting or assessing the sensitivity of a subject having a cancer or a malignant tumor to an immunotherapy using a method according to the present invention as described herein above. If the subject is identified as resistant to the proposed immunotherapy, the method further advantageously comprises an additional step of selecting a distinct chemotherapeutic treatment of cancer more appropriate for the subject.

Also herein described is a method for screening or identifying a compound suitable for improving the treatment of a cancer in a subject having a cancer or a malignant tumor, said method comprising determining the ability of a test compound to modify the expression of at least one of the herein described biomarkers of response or resistance to immunotherapy, or compensate an abnormal expression thereof.

A further embodiment relates to a kit for predicting, assessing or monitoring the sensitivity of a subject having a cancer or a malignant tumor to a cancer therapy, wherein the kit comprises, as detection means, possibly in suitable container means, at least two agents, each of said agents specifically recognizing one of the herein described biomarkers. These at least two agents are typically at least two antibodies selected from the group consisting of an antibody specific to PD-1⁺CD4⁺ T cells, CD8+ T cells and CD25⁺CD127⁻CD4⁺ T cells, CD95⁺CD4⁺ T cells, CD95⁺CD8⁺ T cells, PD-L1⁺CD4⁺ T cells, PD-L1⁺CD8⁺ T cells, CLA⁺CD8⁺ TEM cells, CD137L⁺CD4⁺ T cells, CD137L⁺CD8⁺ T cells, CD137⁺CD4⁺ T cells and CD137⁺CD8⁺ T cells, and, optionally, a leaflet providing the corresponding reference expression levels.

The kit may also comprise a positive control or several positive controls that can be used to determine whether a given agent is capable of specifically recognizing its corresponding biomarker. The kit may also include other reagents that allow visualization or other detection of anyone of the herein described biomarkers, such as reagents for colorimetric or enzymatic assays.

### DETAILED DESCRIPTION OF THE INVENTION

Inventors herein identify predictive biomarkers that are able to secure identification of cancer patients proned to respond or resist to a proposed immunotherapy.

A comprehensive dynamic and functional immunophenotyping gathering 779 blood and tumor parameters was first performed by inventors in 37 stage III melanoma patients for whom ex vivo responses of tumors to monoclonal therapeutic antibodies (mAb) targeting four axis (PD-1/PDL-1, CTLA-4, CD137/CD137L, TIM3) and their combination were tested. Inventors in particular discovered that high expression levels of CD95 as well as PD-L1 on circulating CD4⁺ and CD8⁺ T cells predicted resistance to anti-CTLA-4 Ab in the herein described ex vivo assays and in ipilimumab-treated patients, and that high expression levels of CD137L on circulating CD4⁺ and CD8⁺ T cells predicted resistance to the combination of anti-CTLA4 and anti-PD1 mAbs in said ex vivo assays.

A first object of the invention is an in vitro or ex vivo method of predicting, assessing or monitoring the sensitivity of a subject having a cancer to a proposed immunotherapy. The method typically comprises a step a) of determining, in a biological sample from said subject, the presence, absence or expression level of at least one biomarker, for example at least two biomarkers, and when the expression level is determined a step b) of comparing said expression level to reference expression level(s) or to reference expression ratio(s), thereby predicting, assessing or monitoring whether the subject having a cancer is responsive or resistant to the proposed immunotherapy.

The immunotherapy (also herein identified as “chemotherapeutic drug” or “chemotherapeutic agent”) is typically selected from an antibody, preferably a monoclonal antibody, a chemokine and a cytokine.

The monoclonal antibody can be advantageously selected from anti-PD-1 monoclonal antibody, anti-PD-L1 (ligand) monoclonal antibody, anti-CTLA-4 monoclonal antibody, anti-CD137 monoclonal antibody, anti-CD137L (ligand) monoclonal antibody, and anti-TIM3 monoclonal antibody.

Relevant examples of anti-PD-1 monoclonal antibodies are nivolumab (BMS-936558, MDX-1106 or ONO-4538, Bristol-Myers Squib), pembrolizumab, also known as lambrolizumab (MK-3475, Merck), pidilizumab (formerly CT-011, CureTech Ltd). Preferred examples are nivolumab and pembrolizumab.

Relevant examples of anti-PD-L1 monoclonal antibodies are atezolizumab (MPDL 3280A, Genentech), BMS 936559 or MDX-1105 (Bristol-Myers Squibb), durvalumab (MEDI4736, Medlmmune LLC), avelumab (MSB0010718C, Merck Serono). A preferred example is atezolizumab.

Relevant examples of anti-CTLA-4 monoclonal antibodies are ipilimumab (Yervoy or MDX-010 Bristol-Myers Squibb), tremelimumab (formerly ticilimumab or CP-675,206, Pfizer). Preferred examples are ipilimumab and tremelimumab.

Relevant examples of anti-CD137 monoclonal antibodies is urelumab (BMS-663513, Bristol-Myers Squibb). A preferred example is urelumab.

A relevant example of anti-TIM3 monoclonal antibodies is MBG453 (Novartis).

Cytokines can be selected from pegylated interferon alpha 2a and alpha 2b, IL-2 (proleukin) and IL-2/mAb complexes (also termed IL-2 complexes or IL-2/anti-IL-2 mAb complexes consisting of IL-2 associated to a particular anti-IL-2 mAb). The cytokine is preferably selected from IFNα2a (ROF) and IL-2.

In a particular embodiment of the invention, the immunotherapy is a combined treatment. Preferred combined immunotherapy can be selected from a combination of anti-PD-1 monoclonal antibody and ROF, a combination of anti-CTLA-4 monoclonal antibody and ROF, a combination of anti-PD-1 and anti-TIM3 monoclonal antibodies, and a combination of anti-PD-1 and anti-CTLA-4 monoclonal antibodies. Other combined immunotherapies can involve anti-KIR, anti-OX40, anti-ICOS, anti-VISTA, anti-TIGIT, anti-CD96 and anti-BTLA for example.

In the present invention, the cancer is a cancer that is usually or conventionally treated with one of the herein above described immunotherapy, preferably with an anti-CTLA-4 monoclonal antibody, with an anti-PD-1 monoclonal antibody or with a combination thereof.

The cancer or tumor is typically selected from melanoma, lung, in particular non small cell lung cancer or small cell lung cancer, head and neck cancer, bladder cancer, in particular a bladder cancer with lymph nodes (LN) metastasis, mesothelioma cancer, oesophagus cancer, stomach cancer, hepatocarcinoma cancer, kidney or renal cancer, and breast cancer, in particular triple negative breast cancer, and more generally any cancer amenable to immune checkpoint blockade or leading to stimulation of the immune system. In a preferred embodiment the cancer is a melanoma, in particular a stage III or a stage IV melanoma, typically a stage IV melanoma affecting at least skin and LN.

The cancer or tumor is preferably selected from melanoma, lung, renal cancer, head and neck cancer, bladder cancer, and is even more preferably a melanoma, in particular a stage III melanoma.

In the context of the present invention, the patient or subject is a mammal. In a particular embodiment, the mammal is a human being, whatever its age or sex. The patient typically has a tumor. Unless otherwise specified in the present disclosure, the tumor is a cancerous or malignant tumor. Preferably the subject is a subject who has not been previously exposed to a treatment of cancer, or a subject who has received a chemotherapeutic drug but who has not been treated with an immunotherapy.

In a preferred embodiment of the present invention, the method of the invention is performed after at least partial, for example total, resection of the cancerous tumor and/or metastases thereof, in the subject. The method can however also be performed on the subject before any surgical step.

In the context of melanoma cancer, a particular subpopulation of subjects is composed of stage III or skin and LN positive stage IV melanoma, typically a subpopulation of subjects having undergone at least partial tumor resection. Another particular subpopulation of subjects is composed of subjects having metastases.

In the context of renal cancer, a particular subpopulation of subjects is composed of clear cell renal cancer. Another particular subpopulation of subjects is suffering of clear cell renal cancer and has not undergone surgery yet. A further particular subpopulation of subjects is suffering of clear cell renal cancer, has metastasis and has undergone surgery. Another particular subpopulation of subjects is composed of subjects having renal cancer, in particular clear cell renal cancer, and metastases.

In the context of lung cancer, a particular subpopulation of subjects is composed of locally advanced, non operable non small cell lung cancer (NSCLC), or metastatic lung cancer. Another particular subpopulation of subjects is composed of subjects having lung cancer, in particular NSCLC, and metastases.

As indicated previously, the method of predicting, assessing or monitoring the sensitivity of a subject having a cancer to a proposed immunotherapy according to the invention comprises a step a) of determining, in a biological sample from the subject, the presence, absence or expression level of at least one biomarker, for example at least two biomarkers.

The biomarker is preferably selected from PD-1⁺CD4⁺ T cells, CD8+ T cells and CD25⁺CD127⁻CD4⁺ T cells, CD95⁺CD4⁺ T cells, CD95⁺CD8⁺ T cells, PD-L1⁺CD4⁺ T cells, PD-L1⁺CD8⁺ T cells, CLA⁺CD8⁺ TEM cells, CD137L⁺CD4⁺ T cells, CD137L⁺CD8⁺ T cells, CD137⁺CD4⁺ T cells and CD137⁺CD8⁺ T cells.

Implementations of the methods of the invention involve obtaining a (biological) sample from a subject. The sample can be a fluid sample and may include any specimen containing immune cells such as blood, lymphatic fluid, spinal fluid, pleural effusion, ascites, or a combination thereof. The biological sample can also be a sample comprising tumor cells. Such a sample can be a tumor biopsy, a whole tumor piece, a tumor bed sample, a metastatic lymph node cells sample, or a combination thereof.

A particular method according to the invention is an in vitro or ex vivo method of predicting, assessing or monitoring the sensitivity of a subject having a cancer to an immunotherapy selected from anti-PD-1 monoclonal antibody, anti-PD-L1 monoclonal antibody, anti-CTLA-4 monoclonal antibody, anti-CD137 monoclonal antibody, anti-CD137L monoclonal antibody, anti-TIM3 monoclonal antibody, IFNα2a (ROF), IL-2, a combination of anti-PD-1 and anti-CTLA-4 monoclonal antibodies, a combination of anti-PD-1 monoclonal antibody and ROF, a combination of anti-CTLA-4 monoclonal antibody and ROF, and a combination of anti-PD-1 and anti-TIM3 monoclonal antibodies, which method comprises a step a) of determining, in a biological sample from said subject which is a blood sample or a sample comprising tumor cells, the presence, absence or expression level of at least one biomarker, for example at least two biomarkers, selected from PD-1⁺CD4⁺ T cells, CD8+ T cells and CD25⁺CD127⁻CD4⁺ T cells, CD95⁺CD4⁺ T cells, CD95⁺CD8⁺ T cells, PD-L1⁺CD4⁺ T cells, PD-L1⁺CD8⁺ T cells, CLA⁺CD8⁺ TEM cells, CD137L⁺CD4⁺ T cells, CD137L⁺CD8⁺ T cells, CD137⁺CD4⁺ T cells and CD137⁺CD8⁺ T cells, and, when the expression level is determined, a step b) of comparing said at least one expression level to a reference expression level or to a reference expression ratio, thereby predicting, assessing or monitoring whether the subject having a cancer is responsive or resistant to the immunotherapy.

By “sensitivity” or “responsiveness” is intended herein the likelihood that a patient will respond to a chemotherapeutic treatment as herein described.

By “resistant” is intended herein the likelihood that a patient will not respond to such a chemotherapeutic treatment.

Predictive methods of the invention can advantageously be used clinically to make treatment decisions by choosing as soon as possible the most appropriate treatment modalities for a particular patient and limit toxicities classically associated to immunotherapy.

If the subject is identified, using a method according to the present invention, as resistant to a particular treatment of cancer, the method advantageously further comprises a step of selecting a distinct cancer treatment, for example a distinct immunotherapy typically involving a “compensatory molecule” to be used alone or in combination with the originally preselected chemotherapeutic drug or with a distinct chemotherapeutic drug, as the appropriate therapeutic treatment of cancer for the subject.

Preferably, the step of determining the presence, absence or expression level of at least one biomarker, for example at least two biomarkers, in a biological sample of the subject is performed before any immunotherapeutic treatment step.

In a particular method of the invention, the at least one biomarker, for example at least two biomarkers, is(are)selected from PD-1⁺CD4⁺ T cells, CD8+ T cells and CD25⁺CD127⁻CD4⁺ T cells, CD95⁺CD4⁺ T cells, CD95⁺CD8⁺ T cells, PD-L1⁺CD4⁺ T cells, PD-L1⁺CD8⁺ T cells, CLA⁺CD8⁺ TEM cells, CD137L⁺CD4⁺ T cells, CD137L⁺CD8⁺ T cells, CD137⁺CD4⁺ T cells and CD137⁺CD8⁺ T cells, and the step of determining the presence, absence or expression level of the biomarker(s) in a biological sample of the subject is performed before any immunotherapeutic treatment step, and optionally after at least partial tumor resection in the subject.

In a particular embodiment, typically when CLA⁺CD8⁺ TEM cells are used as a biomarker, this step can be performed three weeks after the first administration (typically injection) of an immunotherapeutic drug (anti-CTLA4 monoclonal Ab, for example ipilimumab) to the subject. This step can also be performed after tumor surgical resection.

In a particular embodiment, the method according to the present invention is an in vitro or ex vivo method of assessing, predicting or monitoring the sensitivity of a subject having a melanoma, preferably a stage III melanoma, and the immunotherapy is selected from anti-CTLA-4 monoclonal antibody, anti-PD-1 monoclonal antibody and combination thereof.

Herein described is thus an in vitro or ex vivo method of predicting, assessing or monitoring the sensitivity of a subject having a cancer to an immunotherapy selected from anti-PD-1 monoclonal antibody, anti-PD-L1 monoclonal antibody, anti-CTLA-4 monoclonal antibody, and a combination of anti-PD-1 and anti-CTLA-4 monoclonal antibodies, wherein the cancer is a melanoma, in particular a stage III melanoma, and the method comprises a step a) of determining, in a biological sample from said subject which is a blood sample or a sample comprising tumor cells, the presence, absence or expression level of at least one biomarker, for example at least two biomarkers, selected from PD-1⁺CD4⁺ T cells, CD8+ T cells and CD25⁺CD127⁻CD4⁺ T cells, CD95⁺CD4⁺ T cells, CD95⁺CD8⁺ T cells, PD-L1⁺CD4⁺ T cells, PD-L1⁺CD8⁺ T cells, CLA⁺CD8⁺ TEM cells, CD137L⁺CD4⁺ T cells, CD137L⁺CD8⁺ T cells, CD137⁺CD4⁺ T cells and CD137⁺CD8⁺ T cells, and, when the expression level is determined, a step b) of comparing said at least one expression level to a reference expression level or to a reference expression ratio, thereby predicting, assessing or monitoring whether the subject having a cancer is responsive or resistant to the immunotherapy.

Typically, the “reference value” or “reference expression level” is the concentration of the biomarker in a control sample derived from one or more subjects (reference population) having a cancer, and is typically the median value obtained from the reference population. The reference value typically varies in a range of values defined for a given population. The reference value can further be a ratio involving two distinct biomarkers or a % or proportion of one several biomarkers in a control sample.

In a particular embodiment, when the patient is bearing anyone of a herein described cancer, preferably a melanoma cancer, typically a stage III melanoma, and when the proposed (candidate) immunotherapy or immunotherapeutic drug is the anti-PD-1 monoclonal antibody, the method of the invention of predicting, assessing or monitoring the sensitivity of a subject having a cancer to the immunotherapy comprises a step a) of determining, in a blood sample of the subject, the expression level of PD-1⁺CD4⁺ T cells, and a step b) of comparing said PD-1⁺CD4⁺ T cells level to a PD-1⁺CD4⁺ T cells reference expression level, an expression level of PD-1⁺CD4⁺ T cells above the PD-1⁺CD4⁺ T cells reference expression level being indicative of sensitivity of the subject to the immunotherapy and an expression level of PD-1⁺CD4⁺ T cells below the PD-1⁺CD4⁺ T cells reference expression level being indicative of resistance of the subject to the immunotherapy, and/or a step a′) of determining, in a blood sample of the subject, the expression levels of CD8⁺ T cells and of CD25⁺CD127⁻CD4⁺ T cells, and a step b′) of determining the ratio of CD8⁺ T cells/CD25⁺CD127⁻CD4⁺ T cells, a ratio above the reference expression ratio being indicative of sensitivity of the subject to the immunotherapy and a ratio below the reference expression ratio being indicative of resistance of the subject to the immunotherapy.

Typically, the PD-1⁺CD4⁺ T cells reference expression level is the percentage of CD4⁺ T cells expressing PD-1, an expression level of PD-1⁺CD4⁺ T cells in the subject corresponding to a percentage of CD4⁺ T cells expressing PD-1 above 21.06% being indicative of sensitivity of the subject to the immunotherapy, and an expression level of PD-1⁺CD4⁺ T cells in the subject corresponding to a percentage of CD4⁺ T cells expressing PD-1 below 7.45% being indicative of resistance of the subject to the immunotherapy.

Typically, a ratio above 5.4 is indicative of sensitivity of the subject to the immunotherapy and a ratio below 2.8 is indicative of resistance of the subject to the immunotherapy.

A typical cut-off percentage used to determine whether a subject is sensitive or resistant will be comprised in the range [7.45%-21.06%]. As well, a typical cut-off ratio used to determine whether a subject is sensitive or resistant will be comprised in the range [2.8-5.4].

In another particular embodiment, when the patient is bearing anyone of a herein described cancer, preferably a melanoma cancer, typically a stage III melanoma, and when the proposed (candidate) immunotherapy or immunotherapeutic drug is the anti-CTLA-4 monoclonal antibody, the method of the invention of predicting, assessing or monitoring the sensitivity of a subject having a cancer to the immunotherapy comprises a step a) of determining, in a biological sample of the subject, the expression level of CD95⁺CD4⁺ T cells, of determining in a blood sample of the subject the expression level of CD95⁺CD8⁺ T cells, of determining in a blood sample of the subject the expression level of PD-L1⁺CD4⁺ T cells, and/or of determining in a blood sample of the subject the expression level of PD-L1⁺CD8⁺ T cells, and a step b) of comparing said levels to their respective reference expression levels, an expression level above the reference expression level being indicative of resistance of the subject to the immunotherapy, and an expression level below the reference expression level being indicative of sensitivity of the subject to the immunotherapy.

Typically, the CD95⁺CD4⁺ T cells reference expression level is the percentage of CD4⁺ T cells expressing CD95, an expression level of CD95⁺CD4⁺ T cells in the subject corresponding to a percentage of CD4⁺ T cells expressing CD95 above 70.80% in a sample comprising tumor cells or above 68.1% in a blood sample being indicative of resistance of the subject to the immunotherapy, and an expression level of CD95⁺CD4⁺ T cells in the subject corresponding to a percentage of CD4⁺ T cells expressing CD95 below 43.79% in a sample comprising tumor cells or below 48.5% in a blood sample being indicative of sensitivity of the subject to the immunotherapy. A typical cut-off percentage used to determine whether a subject is sensitive or resistant will be comprised in the range [43.79%-70.80%] in a sample comprising tumor cells. As well, percentage used to determine whether a subject is sensitive or resistant will be comprised in the range [48.5%-68.1%] in a blood sample.

Typically, the CD95⁺CD8⁺ T cells reference expression level is the percentage of CD8⁺ T cells expressing CD95, an expression level of CD95⁺CD8⁺ T cells in the subject corresponding to a percentage of CD8⁺ T cells expressing CD95 above 74.48% being indicative of resistance of the subject to the immunotherapy, and an expression level of CD95⁺CD8⁺ T cells in the subject corresponding to a percentage of CD8⁺ T cells expressing CD95 below 44.13% being indicative of sensitivity of the subject to the immunotherapy.

A typical cut-off percentage used to determine whether a subject is sensitive or resistant will be comprised in the range [44.13%-74.48%].

Typically, the PD-L1⁺CD4⁺ T cells reference expression level is the percentage of CD4⁺ T cells expressing PD-L1, an expression level of PD-L1⁺CD4⁺ T cells in the subject corresponding to a percentage of CD4⁺ T cells expressing PD-L1 above 27.76% being indicative of resistance of the subject to the immunotherapy, and an expression level of PD-L1⁺CD4⁺ T cells in the subject corresponding to a percentage of CD4⁺ T cells expressing PD-L1 below 6.66% being indicative of sensitivity of the subject to the immunotherapy. A typical cut-off percentage used to determine whether a subject is sensitive or resistant will be comprised in the range [6.66%-27.76%]. The PD-L1⁺CD8⁺ T cells reference expression level is the percentage of CD8⁺ T cells expressing PD-L1, an expression level of PD-L1⁺CD8⁺ T cells in the subject corresponding to a percentage of CD8⁺ T cells expressing PD-L1 above 21.45% being indicative of resistance of the subject to the immunotherapy, and an expression level of PD-L1⁺CD8⁺ T cells in the subject corresponding to a percentage of CD8⁺ T cells expressing PD-L1 below 2.53% being indicative of sensitivity of the subject to the immunotherapy. A typical cut-off percentage used to determine whether a subject is sensitive or resistant will be comprised in the range [2.53%-21.45%].

A particular method herein described wherein the immunotherapy is anti-CTLA-4 monoclonal antibody is a method comprising a step of determining the expression levels of CD95⁺CD4⁺ T cells and PD-L1⁺CD8⁺ T cells in a blood sample of the subject, wherein an expression level of CD95⁺CD4⁺ T cells in the subject corresponding to a percentage of CD4⁺ T cells expressing CD95 above 70% together with an expression level of PD-L1⁺CD8⁺ T cells in the subject corresponding to a percentage of CD8⁺ T cells expressing PD-L1 above 11% is indicative of resistance of the subject to the immunotherapy.

In a further particular embodiment, when the patient is bearing anyone of a herein described cancer, preferably a melanoma cancer, typically a stage III or stage IV melanoma, and when the proposed (candidate) immunotherapy or immunotherapeutic drug is the anti-CTLA-4 monoclonal antibody, the method of the invention of predicting, assessing or monitoring the sensitivity of a subject having a cancer to the immunotherapy comprises a step a) of determining, in a blood sample of the subject three weeks after the first injection of the anti-CTLA4 monoclonal antibody, the percentage and/or absolute number of CLA⁺CD8⁺ TEM cells, and a step b) of comparing said percentage and/or absolute number with a reference percentage and/or absolute number of CLA⁺CD8⁺ TEM cells, a percentage and/or absolute number above the reference percentage and/or absolute number being indicative of sensitivity of the subject to the immunotherapy, and a percentage and/or absolute number below the reference percentage and/or absolute number being indicative of resistance of the subject to the immunotherapy.

Typically, a percentage of CLA⁺CD8⁺ TEM cells above 26.9 and/or absolute number above 33 cells per mm³ is indicative of sensitivity of the subject to the immunotherapy and a percentage of CLA⁺CD8⁺ TEM cells below 6 and/or absolute number below 14 cells per mm³ is indicative of resistance of the subject to the immunotherapy.

A typical cut-off percentage used to determine whether a subject is sensitive or resistant will be comprised in the range [6%-26.9%] and/or a typical cut-off absolute number used to determine whether a subject is sensitive or resistant will be comprised in the range [14-33].

In another particular embodiment, when the patient is bearing anyone of a herein described cancer, preferably a melanoma cancer, typically a stage III melanoma, and when the proposed (candidate) immunotherapy or immunotherapeutic drug is a combination of anti-PD-1 and anti-CTLA-4 monoclonal antibodies, the method of the invention of predicting, assessing or monitoring the sensitivity of a subject having a cancer to the immunotherapy comprises a step a) of determining, in a blood sample of the subject, the expression level of CD137L⁺CD4⁺ T cells, and/or of determining in a blood sample of the subject the expression level of CD137L⁺CD8⁺ T cells, and a step b) of comparing said level(s) to their respective reference expression level(s), an expression level above the reference expression level being indicative of resistance of the subject to the immunotherapy, and an expression level below the reference expression level being indicative of sensitivity of the subject to the immunotherapy.

Typically, the CD137L⁺CD4⁺ T cells reference expression level is the percentage of CD4⁺ T cells expressing CD137L, an expression level of CD137L⁺CD4⁺ T cells in the subject corresponding to a percentage of CD4⁺ T cells expressing CD137L above 25.19% being indicative of resistance of the subject to the immunotherapy, and an expression level of CD137L⁺CD4⁺ T cells in the subject corresponding to a percentage of CD4⁺ T cells expressing CD137L below 9.01% being indicative of sensitivity of the subject to the immunotherapy. A typical cut-off percentage used to determine whether a subject is sensitive or resistant will be comprised in the range [9.01%-25.19%]. The CD137L⁺CD8⁺ T cells reference expression level is the percentage of CD8⁺ T cells expressing CD137L, an expression level of CD137L⁺CD8⁺ T cells in the subject corresponding to a percentage of CD8⁺ T cells expressing CD137L above 16.65% being indicative of resistance of the subject to the immunotherapy, and an expression level of CD137L⁺CD8⁺ T cells in the subject corresponding to a percentage of CD8⁺ T cells expressing CD137L below 7.86% being indicative of sensitivity of the subject to the immunotherapy. A typical cut-off percentage used to determine whether a subject is sensitive or resistant will be comprised in the range [7.86%-16.65%].

In another particular embodiment, when the patient is bearing anyone of a herein described cancer, preferably a melanoma cancer, typically a stage III melanoma, and when the proposed (candidate) immunotherapy or immunotherapeutic drug is a combination of anti-PD-1 and anti-CTLA-4 monoclonal antibodies, the method of the invention of predicting, assessing or monitoring the sensitivity of a subject having a cancer to the immunotherapy comprises a step a) of determining, in a blood sample of the subject, the expression level of CD137⁺CD4⁺ T cells, and/or of determining in a blood sample of the subject the expression level of CD137⁺CD8⁺ T cells, and a step b) of comparing said level(s) to their respective reference expression level(s), an expression level above the reference expression level being indicative of sensitivity of the subject to the immunotherapy, and an expression level below the reference expression level being indicative of resistance of the subject to the immunotherapy.

Typically, the CD137⁺CD8⁺ T cells reference expression level is the percentage of CD8⁺ T cells expressing CD137, an expression level of CD137⁺CD8⁺ T cells in the subject corresponding to a percentage of CD8⁺ T cells expressing CD137 below 3% being indicative of resistance of the subject to the immunotherapy, and an expression level of CD137⁺CD8⁺ T cells in the subject corresponding to a percentage of CD8⁺ T cells expressing CD137 above 3% being indicative of sensitivity of the subject to the immunotherapy.

A further object of the invention is an assay for determining whether a patient is sensitive or resistant to a cancer therapy (also herein identified as ex vivo “mLN assay”), wherein the assay comprises:
a first step wherein suspensions of metastatic lymph nodes samples are incubated ex vivo in duplicate wells, each well of each set of the duplicate being in contact with medium, with a control antibody, or with a test immunotherapeutic antibody defining a cancer therapy, said antibody being preferably selected from an anti-PD-1 monoclonal antibody, an anti-PD-L1 monoclonal antibody, an anti-CTLA-4 monoclonal antibody, an anti-CD137 monoclonal antibody, an anti-CD137L monoclonal antibody, an anti-TIM3 monoclonal antibody, an IFNα2a (ROF), an IL-2, a combination of anti-PD-1 and anti-CTLA-4 monoclonal antibodies, a combination of anti-PD-1 monoclonal antibody and ROF, a combination of anti-CTLA-4 monoclonal antibody and ROF, or a combination of anti-PD-1 and anti-TIM3 monoclonal antibodies, the first set of wells being incubated for 18h-24h, and the second set of wells being incubated for 4 to 5 days,
a second step of measuring T cells, NK cells and/or Treg cells parameters, said parameters consisting in cell biomarker(s) expression, cytokine cell release, interferon γ cell release, chemokine cell release and/or interleukin cell release in the first set of wells, and Ki67 cell expression and Treg cell proportion in the second set of wells, and
a third step of comparing measures obtained in each well with the corresponding measure obtained from the medium and control wells, a 1.5 fold variation or more than a 1.5 fold variation, typically increase, of at least two parameters (“positive parameters”) indicating that the patient is sensitive to the cancer therapy.

As an exception among measured parameters herein identified, a 1.5 fold decrease or less than a 1.5 fold decrease of CD4⁺FoxP3⁺ Treg level (when compared to the CD4⁺FoxP3⁺ Treg level obtained from the medium and control wells) is typically considered as variation indicating that the patient is sensitive to the cancer therapy when combined to at least one other “positive parameter”).

Typically, a cell surface biomarker expression can easily be determined by FACS and a molecule cell release can easily be determined by ELISA as further explained below.

Cell biomarker(s) the expression of which can be measured in the herein above described “mLN assay” can be selected from anyone of the cell biomarkers identified on FIGS. 3-5, such as Foxp3, Ki67, IFNγ, TNFα, as well as any combination thereof.

Cytokine the expression of which can be measured in the herein above described “mLN assay” can be selected from GCSF, IFNγ, TNFα and any combination thereof.

Chemokine the expression of which can be measured in the herein above described “mLN assay” can be selected from CCL2, CCL3, CCL4, CCL5, CXCL8, CXCL9, CXCL10 and any combination thereof.

Interleukin the expression of which can be measured in the herein above described “mLN assay” can be selected from IL1B, IL2, IL6, IL10, IL12p70, IL13 and any combination thereof.

In some embodiments of the invention, identification of a biomarker of interest involves use of at least one binding agent. Furthermore, it is contemplated that a binding agent may be specific or not to the considered biomarker. For example, the CD95⁺CD4⁺ T cells binding agent may bind to a part of CD95 (e.g. an epitope) that is not available depending on whether it is expressed by/bound to circulating CD95⁺CD4⁺ T cells from a fluid sample or by CD95⁺CD4⁺ T cells from a biological sample comprising tumor cells as previously described. Alternatively, different conformations may serve the basis for binding agents capable of distinguishing between similar biomarkers.

The binding agent is typically a polypeptide. The polypeptide is, in particular embodiments, an antibody. In further embodiments, the antibody is a monoclonal antibody. The antibody can be bi-specific, recognizing two different epitopes. The antibody, in some embodiments, immunologically binds to more than one epitope from the same biomarker. In some embodiments of the invention, the binding agent is an aptamer.

In some embodiments of the invention, the binding agent is labeled. In further embodiments, the label is radioactive, fluorescent, chemiluminescent, an enzyme, or a ligand. It is also specifically contemplated that a binding agent is unlabeled, but may be used in conjunction with a detection agent that is labeled. A detection agent is a compound that allows for the detection or isolation of itself so as to allow detection of another compound that binds, directly or indirectly. An indirect binding refers to binding among compounds that do not bind each other directly but associate or are in a complex with each other because they bind the same compounds or compounds that bind each other.

When CD95 (Fas) is to be detected, the antibody to be used can be DX2 (BD Biosciences in APC—reference: 558814).

When CD137L (4-1BBL) is to be detected, the antibody to be used can be C65-485 (BD Biosciences in PE—Reference: 559446).

When CD137 (4-1BB) is to be detected, the antibody to be used can be 4B4-1 (Biolegend—Reference: 309810).

When CD4 is to be detected, the antibody to be used can be SK3 (BD Biosciences in PerCP—Reference: 3457703).

When CD8 is to be detected, the antibody to be used can be RPA-T8 (BD Biosciences in FITC—Reference: 555366).

When PD-1 is to be detected, the antibody to be used can be PD1.3.5 (Beckman Coulter in Pe-Cy7—Reference: A78885).

When PD-L1 (CD274) is to be detected, the antibody to be used can be 29E.2A3 (BioLegend in APC—Reference: 329708).

When CLA is to be detected, the antibody to be used can be HECA-452 (BD Biosciences in FITC—Reference: 561987).

When CD25 is to be detected, the antibody to be used can be M-A251 (BD Biosciences in PE—Reference: 555432).

When CD127 is to be detected, the antibody to be used can be MB15-18C9 (Miltenyi Biotec in APC—Reference: 130-094-890).

Other embodiments of the invention involve a second binding agent in addition to a first binding agent. The second binding agent may be any of the entities discussed above with respect to the first binding agent, such as an antibody. It is contemplated that a second antibody may bind to the same of different epitopes as the first antibody. It is also contemplated that the second antibody may bind the first antibody or another epitope than the one recognized by the first antibody.

As discussed earlier, binding agents may be labeled or unlabeled. Any polypeptide binding agent used in methods of the invention may be recognized using at least one detection agent. A detection agent may be an antibody that binds to a polypeptide binding agent, such as an antibody. The detection agent antibody, in some embodiments, binds to the Fc-region of a binding agent antibody. In further embodiments, the detection agent is biotinylated, which is incubated, in additional embodiments, with a second detection agent comprising streptavidin and a label. It is contemplated that the label may be radioactive, fluorescent, chemiluminescent, an enzyme, or a ligand. In some cases, the label is an enzyme, such as horseradish peroxidase.

The present invention also covers methods involving using flow cytometry or ELISA assay to detect biomarkers.

In some embodiments, the selected flow cytometry technology is FACS (Fluorescence-activated cell sorting). FACS can be used for distinguishing and separating into two or more containers specific cells from a heterogeneous mixture of biological cells, based upon the specific light scattering and fluorescent characteristics of each cell.

In other embodiments, the ELISA assay is a sandwich assay. In a sandwich assay, more than one antibody will be employed. Typically ELISA method can be used, wherein the wells of a microtiter plate are coated with a set of antibodies which recognize the protein of interest. A sample containing or suspected of containing the protein of interest is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

In the methods herein described of predicting, assessing or monitoring the sensitivity of a subject having a cancer to an immunotherapy as well as in the methods herein described of selecting an appropriate chemotherapeutic treatment, any classical method known by the skilled person of determining the presence or measuring the expression level of a compound of interest, such as typically FACS, ELISA and radioimmunoassay can be used.

A method of selecting an appropriate, preferably optimal, therapeutic treatment of cancer for a subject having a cancer as herein described, is in addition herein described, as well as appropriate chemotherapeutic treatment involving for example compensatory molecules for use in such a treatment of cancer, possibly in combination with the preselected chemotherapeutic drug, in a subject identified, using a method as herein described, as resistant to said preselected chemotherapeutic drug.

Also herein described is thus a method of selecting an appropriate chemotherapeutic treatment for a subject, which method comprises a step of assessing the sensitivity of a subject having a cancer to a proposed immunotherapy using a method according to the present invention as described herein above.

If the subject is identified as sensitive to the proposed immunotherapy, this means that said immunotherapy is an appropriate chemotherapeutic treatment for the subject.

If the subject is identified as resistant to the proposed immunotherapy, this means that said immunotherapy will not be efficient in the subject and will in addition possibly generate unwanted deleterious side effects in the subject. In such circumstances, the method further advantageously comprises an additional step of selecting a distinct chemotherapeutic treatment of cancer more appropriate for the subject.

For subject suffering of a melanoma, when anti-PD-1 monoclonal antibody is not efficient, or not efficient alone, in the subject, the distinct chemotherapeutic treatment can be a compound selected from any other immunostimulatory monoclonal antibody such as an antibody targeting CTLA4, TIM3, LAG3, VISTA, BTLA, CD137, OX40, ICOS, B7-H3, B7-H4, KIR, IDO, or TIGIT, and any combination thereof; or a combination of the anti-PD-1 monoclonal antibody and of such a distinct compound.

Methods of screening for candidate therapeutic agents for preventing or treating cancer are also included as part of the invention. The method is typically a method which is performed in vitro or ex vivo. When performed ex vivo, it can be performed for example on a sample from a subject who has been administered with a test compound.

A method herein described is typically a method for screening or identifying a compound suitable for improving the treatment of a cancer in a subject having a cancer, said method comprising determining the ability of a test compound to modify the expression of at least one of the herein described biomarkers of response or resistance to immunotherapy, or compensate an abnormal expression thereof.

The present invention also includes kits for predicting, assessing or monitoring the sensitivity of a subject having a cancer to a cancer therapy, in particular an immunotherapy, wherein the kit comprises, as detection means, possibly in suitable container means, at least two agents, for example three, four or five agents, each of said agent specifically recognizing one of the herein described biomarkers. These at least two agents are typically at least two antibodies selected from the group consisting of an antibody specific to PD-1⁺CD4⁺ T cells, CD8+ T cells and CD25⁺CD127⁻CD4⁺ T cells, CD95⁺CD4⁺ T cells, CD95⁺CD8⁺ T cells, PD-L1⁺CD4⁺ T cells, PD-L1⁺CD8⁺ T cells, CLA⁺CD8⁺ TEM cells, CD137L⁺CD4⁺ T cells, CD137L⁺CD8⁺ T cells, CD137⁺CD4⁺ T cells or CD137⁺CD8⁺ T cells and, optionally, a leaflet providing the corresponding reference expression levels.

In further embodiments, the binding agent is labeled or a detection agent is included in the kit. It is contemplated that the kit may include one, at least one or several, biomarker binding agents attached to a non-reacting solid support, such as a tissue culture dish or a plate with multiple wells. It is further contemplated that such a kit includes one or several detectable agents in certain embodiments of the invention. In some embodiments, the invention concerns kits for carrying out a method of the invention comprising, in suitable container means: (a) agent(s) that specifically recognizes all or part of a given biomarker; and, (b) at least one positive control, for example two positive controls, that can be used to determine whether the agent is capable of specifically recognizing all or part of said given biomarker. The kit may also include other reagents that allow visualization or other detection of the biomarkers, such as reagents for colorimetric or enzymatic assays.

The invention will be further illustrated by the following figures and examples. However, these examples and figures should not be interpreted in any way as limiting the scope of the present invention.

### FIGURES

FIG. 1. Experimental setting of the ex vivo “mLN assay”.

Metastatic lymph nodes (mLN) containing 4-98% melanoma tumor cells (CD45⁺ cells) were resected, freshly mechanically and enzymatically dissociated using the Gentle MACs Miltenyi equipment for 1 hour at 37° C. under rotation (2 incubation steps of 30 minutes). Whole cell suspensions were incubated in duplicate (one for the 18-24 hrs readout and one for the 4-5 days readout) wells at 0.3×10⁶/ml with medium, versus isotype control Ab or a series of antagonistic or agonistic mAb or combinations or recombinant cytokines as outlined. The ex vivo stimulation lasted 18-24 hrs (except in 2 cases where it lasted 48 hrs) before flow cytometric analyses of live CD45⁺ cells, within CD3⁺CD4⁺, CD3⁺CD8⁺ or CD3⁻CD56⁺ cell gates for intracellular staining of Th1 cytokines (IFNγ, TNFα) after a final 3-5 hr activation with PMA, ionomycine and GolgiStop. The 18-24 hr cytokine release was monitored by commercial ELISA or multiplex arrays. The day 4-5 time point was crucial for monitoring proliferation by flow cytometric analyses of Ki67 on T, NK and Tregs populations.

FIG. 2. Inter-individual variations in specimen handling and data harvesting in two patients lesions.

Exemplification of positive scoring in several immunometrics for two patients using different ex vivo stimulations performed in parallel and two independent experiments to cross-validate the findings and data mining approach. Flow cytometry dot plots are shown for the experiments performed on one patient (A) and CXCL10 dosage by ELISA on patients' lesions (B-E).

FIG. 3. Heatmap data sheets segregating responding versus non responding patient lesions in each stimulation axis (IFNα2a, IL-2)

Heatmap depicting the immunometrics scoring in the IFNα2a and IL2 simulation axes.

For improved readability of the scoring heatmap, patients are segregated according to their response lesions for the IFNα2a and IL-2 stimulation axes. Each column represents a patient and each row a parameter (an immunometric). Cells are coded in black if the fold change of the marker is above 1.5 (compared to the control) and in grey otherwise. White cells indicate that the marker could not be evaluated for the corresponding patient. The total sums of positive immunometrics (out of the number of evaluated immunometrics) are shown for each marker (y-axis) or patient (x-axis). The most representative data for each axis are also plotted on a graph appearing on FIG. 6.

FIG. 4. Heatmap data sheets segregating responding versus non responding patient lesions in each stimulation axis (anti-CTLA-4, anti-PD-1)

Heatmap depicting the immunometrics scoring in the anti-PD-1 and anti-CTLA-4 stimulation axes. Each column represents a patient and each line a parameter (an immunometric). Cells are coded in black if the fold change of the marker is above 1.5 (compared to the control) and in grey otherwise. White cells indicate that the marker could not be evaluated for the corresponding patient. The total sums of positive immunometrics (out of the number of evaluated immunometrics) are shown for each marker (y-axis) or patient (x-axis). The most representative data for each axis are also plotted on a graph appearing on FIG. 6.

FIG. 5. Heatmap data sheets segregating responding versus non responding patient lesions in each stimulation axis (anti-CTLA-4+anti-PD-1, anti-CD137 and/or anti-CD137L, anti-TIM3).

Heatmap depicting the immunometrics scoring in the anti-CTLA-4+anti-PD-1, anti-CD137/CD137L and anti-Tim-3 stimulation axes. Each column represents a patient and each line a parameter (an immunometric). Cells are coded in black if the fold change of the marker is above 1.5 (compared to the control) and in grey otherwise. White cells indicate that the marker could not be evaluated for the corresponding patient. The total sums of positive immunometrics (out of the number of evaluated immunometrics) are shown for each marker (y-axis) or patient (x-axis). The most representative data for each axis are also plotted on a graph appearing on FIG. 6.

FIG. 6. Typification of responses for each axis of stimulation.

Summary of FIGS. 3-5 showing mLN responding to each axis (anti-CTLA4, anti-PD-1, anti-TIM3, anti-CD137, anti-CD137L, IFNα2A, IL-2 or combinations thereof) exhibiting specific immunometrics such as activation of effector cells (proliferation or cytokine release) shared by at least 20% patients. M&M detail the experimental settings. Briefly, functional assays used flow cytometry determination of early (18-24 hrs post-stimulation) intracellular cytokine release in T and NK cells, late (day 4-5 post-stimulation) proliferation assays, chemokine and cytokine secretions in the supernatants at 18-24 hrs. A biological response to a given axis was scored “positive” when two independent readouts, reaching a >1.5 fold increase or decrease over two background levels (that of the medium and the isotype control Ab) were achieved.

FIG. 7. Global representation of the patterns of response to individual or combined stimulations for 37 MMel (also herein identified as “MM” or as metastatic melanoma).

A-B Venn diagram representing each stimulating axis alone (A) or in combinations (B) per circle, patients being identified by letters and numbers. C-D Frequencies of lesions that failed to respond to a given axis but could exhibit significant responses to alternative axis of stimulation or combination. The detailed patterns of responses are in Table 2. E Frequencies of patient lesions that failed to respond to a given axis (first bar,) but could exhibit significant responses to alternative axis of stimulation. (second and third bar). For instance (very left bars), in the non-responding lesions (NR) to anti-CTLA4 Ab, inventors annotated the percentages that could respond (or not) to anti-CTLA4+anti-PD1 Ab co-blockade (second bar), among which some of them could respond (or not) to anti-PD1 Ab alone (third bar). The detailed patterns of responses feature in Table 6. −: ND.

FIG. 8. CD95 expression on blood CD4⁺ T cells predict resistance to ipilimumab (anti-CTLA4 Ab).

A. Display of the likelihood ratio test versus the bootstrapped AUC of the markers used to assess the sensitivity to anti-CTLA-4 Ab in the blood (up) and tumor (down) samples. B-C. Expression levels of CD95 on tumor TIL (B) and blood (C) CD4⁺ T cells in lesions responding (R) or not (NR) to the ex vivo mLN assay using anti-CTLA-4 Ab. Each dot represents one patient. The absolute numbers of patients are indicated in parentheses in both groups. Graphs analyzed beta regression and Wilcoxon rank-sum test (left panel) or receiver operating characteristics (ROC) curves alongside the estimated area under the curve (AUC) statistics (right panel). D.

Distributions of CD95 expression on blood CD4⁺ T cells at diagnosis prior to CTLA-4 blockade across patient groups stratified based on objective responses at 3 months during Ipilimumab treatment. Pairwise comparisons were extracted from the beta regression model and presented after adjusting for multiple comparisons (up panel) or ROC curves alongside the estimated AUC statistics (down panel), PD: Progression Disease, SD: Stable Disease, PR: Partial Response, CR: Complete Response. E. Kaplan-Meier curves of the progression-free survival (PFS) (up panel) and overall survival (OS) (down panel) segregating a cohort of MM patients according to the median values (above in grey and below in black lines) of CD95 expression on CD4⁺ T cells. F. Kaplan-Meier curves of the OS (surviving patients) (up panel) and PFS (receive free patient) (down panel) segregating a cohort of MM patients who did not receive Ipilimumab treatment after surgery according to the median values (above in grey and below in black lines) of CD95 expression on CD4⁺ T cells (cohort detailed in Jacquelot et al, JID in press, aimed at analyzing prognostic factors in stage III MM). G. Display of the Wilcoxon rank sum test versus the empirical AUC of the markers used to assess the sensitivity to anti-CTLA-4 Ab in blood (up) or tumor (down) samples. Each dot represents one marker; selected biomarkers are shown with a cross while biomarkers with very low level of expression are shown in grey.

FIG. 9. CD95 expression on CD8⁺ T cells also predicts resistance to CTLA4 blockade.

A-D. Expression levels of CD95 on blood (A-B) and tumor (C-D) CD8⁺ T and TILs respectively in lesions responding (R) or not (NR) to the ex vivo mLN assay in the anti-CTLA4 Ab stimulatory condition. Each dot represents one patient. The absolute numbers of patients are indicated in both groups. p-values obtained by beta regression and Wilcoxon rank-sum test (A,C) or estimates of the AUC under the ROC curves (B,D) tests are shown.

FIG. 10. PD-L1 expression on circulating CD4⁺ and CD8⁺ T cells predicts resistance to ipilimumab.

A-H. Expression levels of PD-L1 on blood (A-D) and tumor (E-H) of CD8⁺ and CD4⁺ T cells and TILs respectively in lesions responding (R) or not (NR) to the ex vivo mLN assay in the anti-CTLA-4 Ab stimulatory condition. Each dot represents one patient. The absolute numbers of patients are indicated in parenthesis in both groups. I. Distribution of PD-L1 expression on blood CD8⁺ T cells at diagnosis prior to CTLA4 blockade across objective response rates at 3 months of ipilimumab (up and down panel), PD: Progression Disease, SD: Stable Disease. J. OS (up panel) and PFS (down panel) segregating the cohort according to the median values (above in grey and below in black lines). K. Correlations between PD-L1 expression (above the median value in grey and below in black lines) on blood CD8⁺ T cells at diagnosis and PFS (receive free patients) (up panel) or OS (surviving patients) (down panel) in stage III MM that did not receive ipilimumab after surgery (cohort detailed in Jacquelot et al, JID in press, aimed at analyzing prognostic factors in stage III MM). p-values obtained by beta regression and Wilcoxon rank-sum test (A,C,E,G,I up) or estimates of the AUC under the ROC curves (B,D,F,H,I down) tests are shown.

FIG. 11. CD95 expressing TEM and TCM are activated and exhausted cells.

(A) Expression of CD95 on various CD4⁺ (A1) and CD8⁺ (A2) T cell subsets (defined using CD45RA, CCR7, CD127 and CD25 markers by flow cytometry analyses) in blood and tumor beds in 7 individuals diagnosed with stage III MMel. (B) Expression of activation and exhaustion markers (indicated in the X axis) gating on CD95⁺ (+) or CD95⁻ (−) CD4⁺ T cells from blood or tumor lesions in 7 individuals. Each dot represents the value of one patient with the number of patients tested indicated in parentheses. p-values from linear mixed effect modeling are indicated. (C) CD95 expression according to PD1 and PD-L1 expression in T cells. Each dot represents the value of one patient with the number of patients tested indicated in parentheses. p-values from Wilcoxon rank sum test are indicated.

FIG. 12. PD-1 and PD-L1 expression on blood T cells and the CD8⁺ T cell/Treg ratio in blood predict responses to aPD-1 Ab in ex vivo mLN assays.

A1 Display of the likelihood ratio test versus the bootstrapped AUC of the markers used to assess the sensitivity to anti-CTLA-4+anti-PD-1 Ab in the blood. A2 Display of the Wilcoxon rank sum test versus the empirical AUC of the markers used to assess the sensitivity to anti-PD-1 mAb in the blood. Each dot represents one marker, selected biomarkers are shown with cross while biomarkers with low level of expression are shown in grey. B-E. Expression levels of PD-1 (B-C) on blood CD4⁺ T cells and CD8/Treg ratio (D-E), expression levels of PD-L1 on CD4⁺ and CD8⁺ T cells (H), in patients lesions responding (R) or not (NR) to the ex vivo mLN assay in aPD-1 mAb stimulatory condition. Each dot represents one patient. The absolute numbers of patients are indicated in both groups. p-values obtained by beta regression and Wilcoxon rank-sum test (B,D) or estimates of the AUC under the ROC curves (C,E) tests are shown. F-G. Kaplan Meier curves of PFS (up panels) or OS (down panels) in stage III MM that did not receive a PD-1 nor a PD-L1 Ab after surgery segregating the cohorts based on median values (above in grey and below in black lines) of PD-1 on CD4⁺ T cells (F) or CD8/Treg (G) in blood at diagnosis (cohort detailed in Jacquelot et al, JID in press, aimed at analyzing prognostic factors in stage III MM).

FIG. 13. CD137L/4-1BBL expression on blood CD4⁺ and CD8⁺ T cells predict resistance to the combination a CTLA-4+a PD-1 Ab.

A1 Display of the likelihood ratio test versus the bootstrapped AUC of the markers used to assess the sensitivity to anti-CTLA-4+anti-PD-1 Ab in the blood. A2 and F. Display of the Wilcoxon rank sum test versus the empirical AUC of the markers used to assess the sensitivity to anti-CTLA-4+anti-PD-1 Abs in the blood (A2) and tumor (F) in the ex vivo mLN assay. Each dot represents one marker, selected biomarkers are shown with a cross while biomarkers with low level of expression are shown in grey. B-C. Expression levels of CD137L/4-1BBL in blood (B) and tumor beds (C) of CD4⁺ T cells in lesions responding (R) or not (NR) to the ex vivo mLN assay in a CTLA-4+a PD-1 Ab stimulatory condition. Each dot represents one patient. The absolute numbers of patients are indicated in both groups in parenthesis. Graphs analyzed with LRT and Wilcoxon (left panels) or ROC curves with AUC (right panels) statistical tests are shown. D-E. Expression levels of CD137L/4-1BBL in blood (D) and tumor beds (E) in CD8⁺ T cells in lesions responding (R) or not (NR) to the ex vivo mLN assay in a CTLA-4+a PD-1 Ab stimulatory condition. Each dot represents one patient. Numbers of patients are indicated for each group in parentheses. p-values obtained by beta regression and Wilcoxon rank-sum test (left panels) or estimates of the AUC under the ROC curves (right panels) tests are shown.

FIG. 14. Ipilimumab-induced CLA⁺ CD8⁺ TEM cells are associated with favorable clinical outcome.

A-B left panels. Absolute numbers (A) and proportions (B) of CLA expressing CD8⁺ TEM cells over time are depicted in a cohort of 47 ipilimumab-treated MM patients then segregated into non-responders (NR) or responders (R) evaluated 3 months (4 injections) after therapy commencement. A-B right panels. ROC curves depicting the predictive properties of each parameter determined after 1 ipilimumab injection and associated area under the curve (AUC). C-D. Idem as A and B but for the absolute number (C) and proportions (D) of CLA expressing CD4⁺ TEM cells. Each point represents one patient specimen, and the total number is indicated for all subpopulations studied. Statistical analyses were performed by logistic regression and adjusted on investigation centers (A-D). p values are indicated.

FIG. 15. CD137/4-1BB expression on blood CD4⁺ and CD8⁺ T cells predicted sensitivity to the combination of anti-CTLA-4+anti-PD-1 Ab in the “ex vivo mLN assay”.

A-D. Expression levels of CD137/4-1BB in blood (A-B) and tumor bed (C-D) of CD4⁺ (A, C) and CD8⁺ T (B, D) cells, respectively, in patients' lesions responding (R) or not (NR) to the ex vivo mLN assay in the anti-CTLA-4+anti-PD1 mAbs stimulatory condition. Each dot represents one patient. The absolute numbers of patients are indicated in both groups in parenthesis. Graphs were analyzed by beta regression and Wilcoxon rank-sum test (left panels) or receiver operating characteristics (ROC) curves alongside the estimated area under the curve (AUC) statistics (right panel).

FIG. 16. 4-1BB expression on blood CD8⁺ T cells predicted resistance and sensitivity to ipilimumab+nivolumab therapies in retrospective analyses.

Prospective analysis of CD137/4-1BB expression on T cells prior to enrollment in a Phase II trial of high risk stage IIIc/IV MMel patients. A-C Distributions of CD137 expression on blood CD8⁺ T cells (A, B) and on blood CD4⁺ T cells (C) at diagnosis prior to PD1/CTLA-4 co-blockade (A and C) or PD1 blockade (B) across patients' groups stratified based on progression [relapse or no evidence of disease (NED)] with a median follow up of 13 months post enrolment in adjuvant therapy for stage III/IV resected MMel. Each dot represents the FACS analysis value of CD137 expression compared with a staining with an isotype control mAb prior to adjuvant therapy. Mean+SEM are represented. Unpaired t-test was applied for the statistical analyses.

FIG. 17. Melanoma patients express higher levels of PD-L1 on circulating T cells than healthy volunteers.

A-C. Percentages of CD95 (a) and/or PD-L1⁺ (b, c) cells among blood CD4⁺ (a, b) or CD8⁺ (c) T cells respectively at baseline prior to ipilimumab. Flow cytometric assessments of the proportions of blood CD3⁺CD4⁺ or CD8⁺ cells expressing PD-L1 after thawing in 8 cohorts of MMel (right columns) except CA and/or JE (not assessed), as well as 10-35 healthy volunteers (, left column). Each dot represents one healthy volunteer or patient. Mean and SEM are represented along with the box plots for each cohort described in Table 7. D. Expression of PD-L1 on CD8⁺ T cells according to the metastatic sites: 1 (skin, mLN, lung mets only), 2 (visceral metastases, soft tissues+/−group 1), 3 (bone metastases and +/−groups 1 and/or 2) and 4 (brain metastases and others). E-F. Spearman correlation between PD-L1⁺/CD8⁺ and PD-L1⁺/CD4⁺ or CD95⁺/CD8⁺ with rho index; each dot representing one patient.

FIG. 18. Predictive values of PD-L1⁺/CD8⁺ and CD95⁺/CD4⁺ for RR to ipilimumab.

A-B Statistical analyses of the clinical relevance of CD95 expression on CD4⁺ T cells as well as PD-L1 on CD8⁺ T cells according to RR (separating PD from SD, PR or CR) were performed in univariate and multivariate regression assays. Each dot represents one patient. The box plots indicating the mean and SEM of values for the binary separation are depicted (cf Table 9). The absolute numbers of patients are indicated in all groups in parentheses.

FIG. 19. Relative risk of death according to PD-L1 or CD95 on T cells.

Overall survival is fulfilled for n=189 patients including 121 events. A. Graphical visualization of the log relative risk according to the biomarkers' scores. PD-L1 expression (as a continuous variable) on CD8⁺ (a, p=0.021) T cells as a function of the relative risk to die under ipilimumab therapy at baseline. B. Kaplan-Meier OS curves segregating the whole cohort according to the median value of PD-L1 expression on blood CD8⁺ T cells at baseline (p=0.011 in multivariate analyses, Table 6). C. Idem for CD95 on CD8⁺ T cells (p=0.056). D. Kaplan-Meier OS curves segregating the whole cohort according to a cut-off value of 70% for the CD95 expression on blood CD4⁺ T cells at baseline (p=0.02 in multivariate analyses, Supplementary Table 5). p values indicated in this legend consider the biomarker as a median (binary) or a continuous variable in multivariate analysis (refer to the Table 6 and Table 10).

FIG. 20. PFS and OS in the 8 cohorts of MMel patients treated with ipilimumab and described in Table 7.

FIG. 21. Importance of the expression of CD95 and PDL1 on blood T cells for the prediction of the overall survival after ipilimumab therapy.

Kaplan-Meier OS curves segregating the whole cohort in 4 arms according to a cut-off value at 70% of CD95 expression on blood CD4⁺ T cells and the median value of PD-L1 expression on blood CD8⁺ T cells before ipilimumab therapy (refer to the Table 6 and Table 10). p-values are indicated.

Throughout this application, various references describe the state of the art to which this invention pertains. The disclosures of these references are hereby incorporated by reference into the present disclosure.

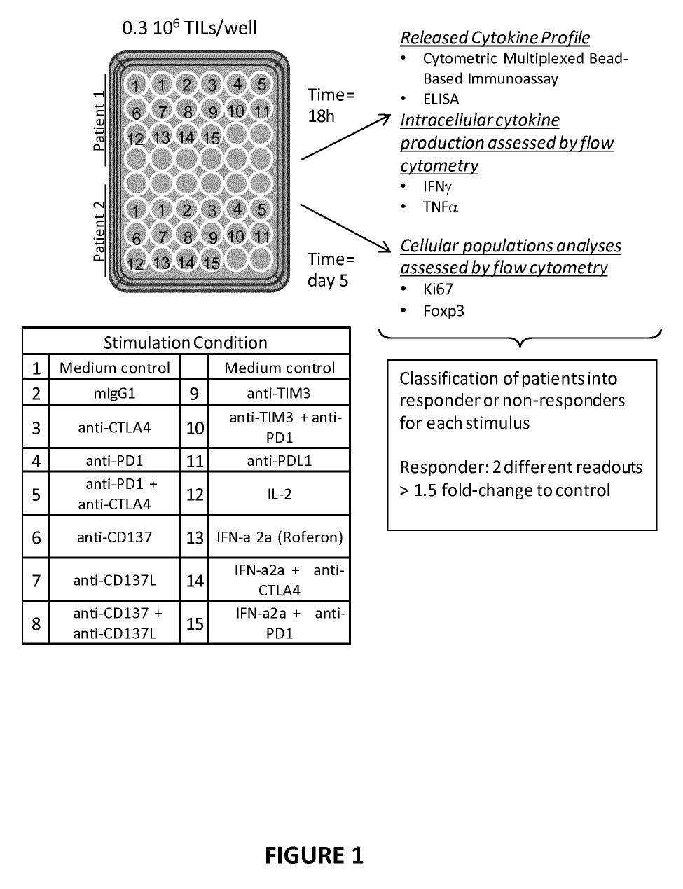

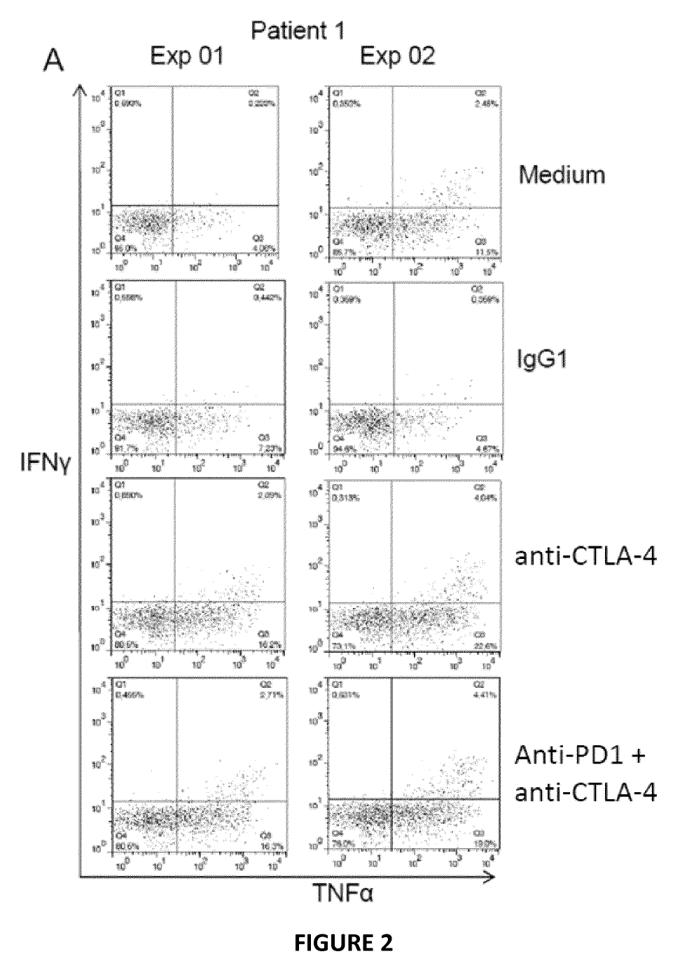

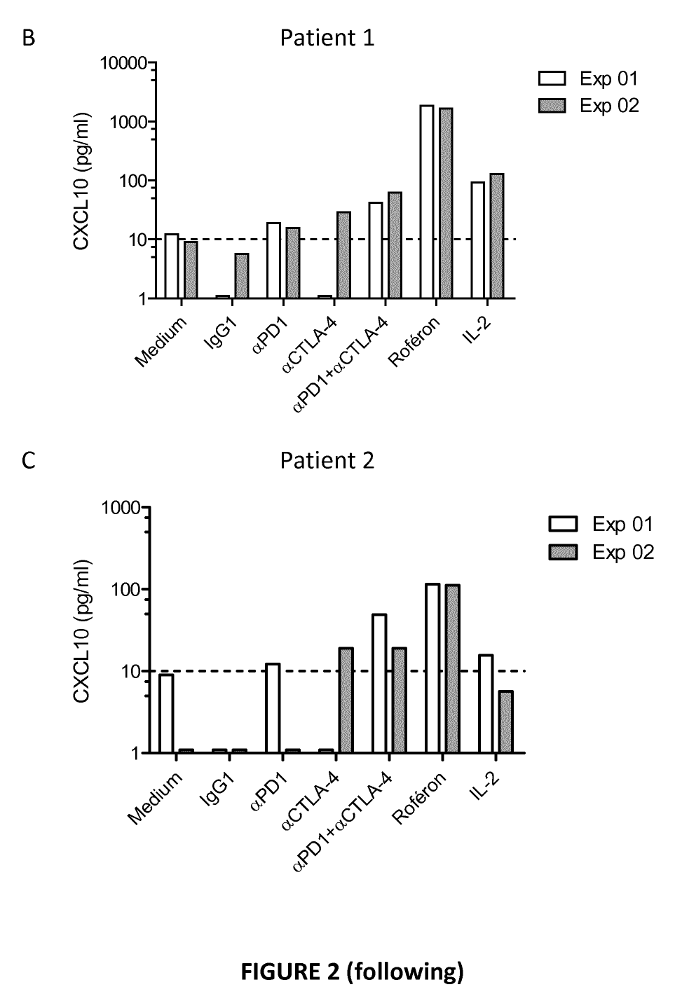

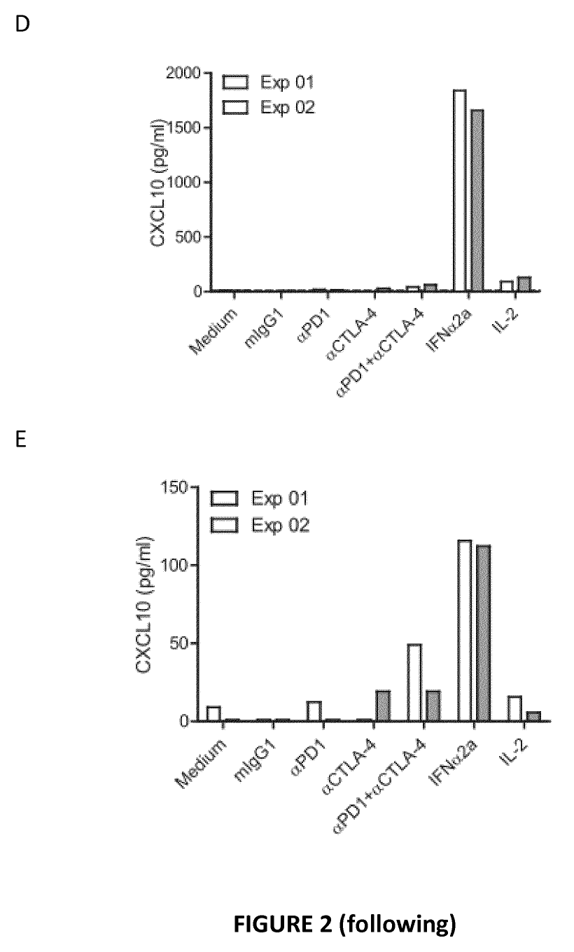

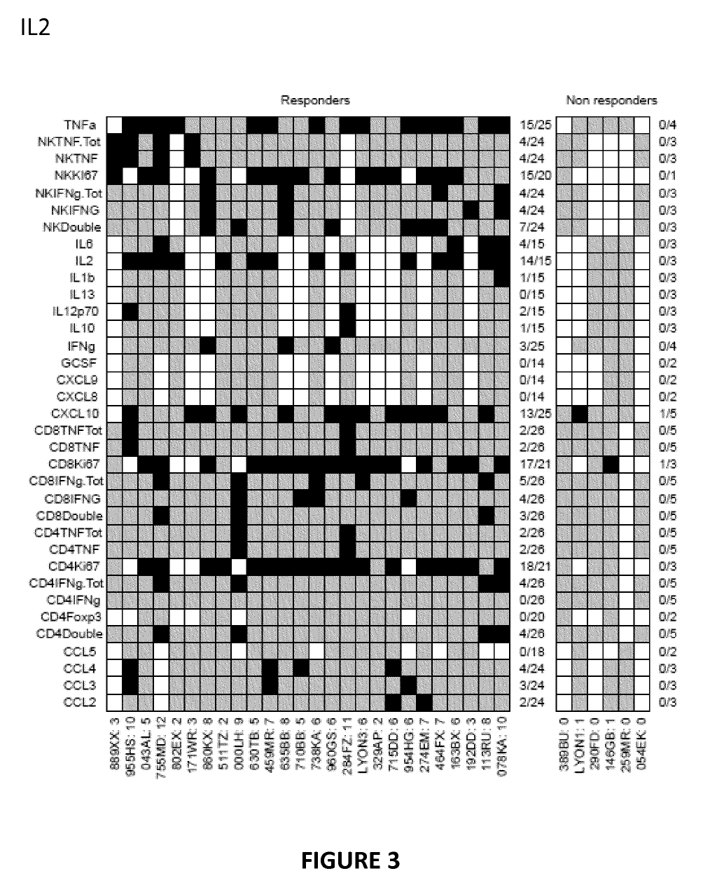

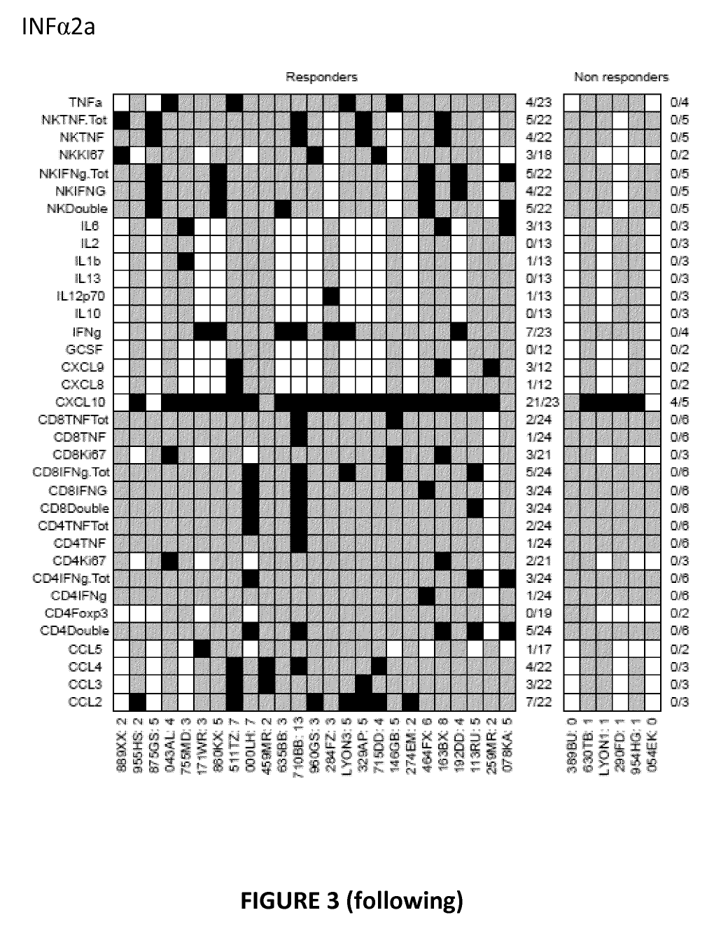

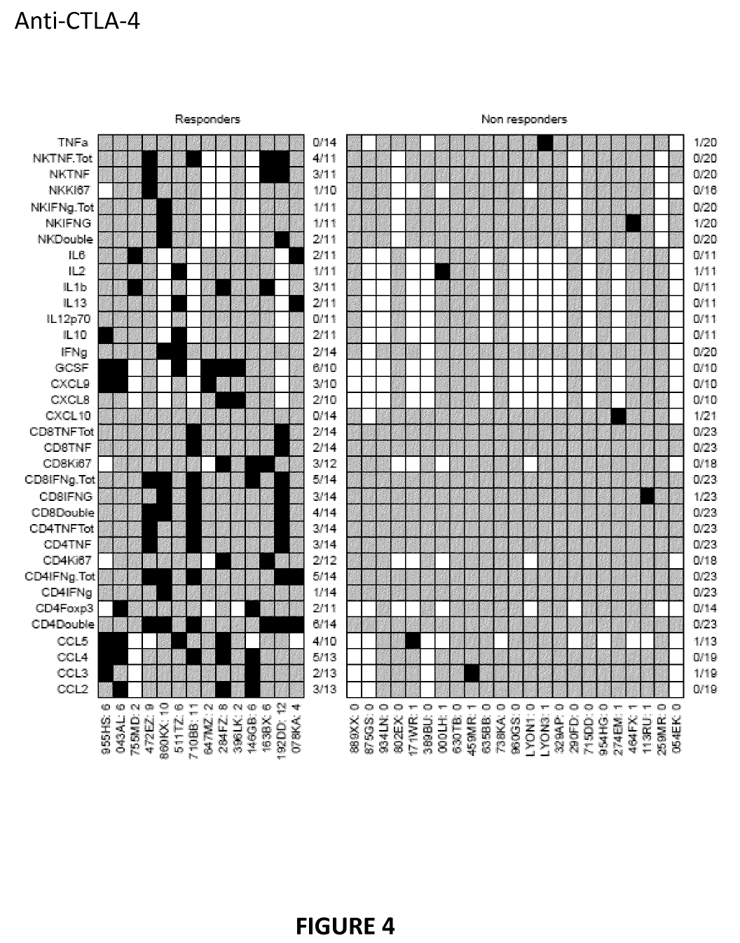

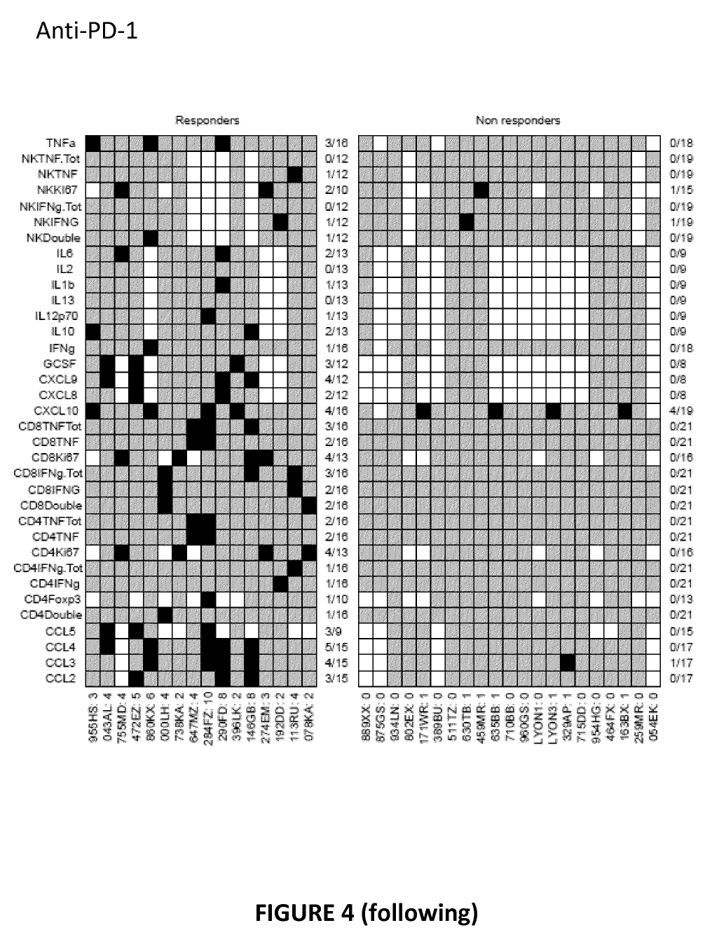

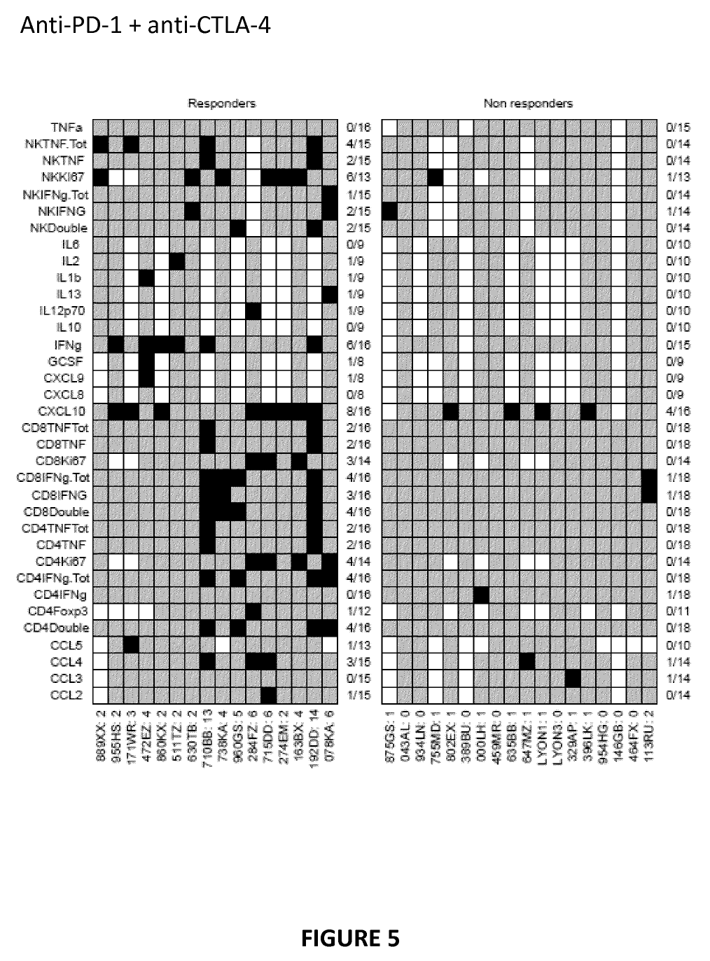

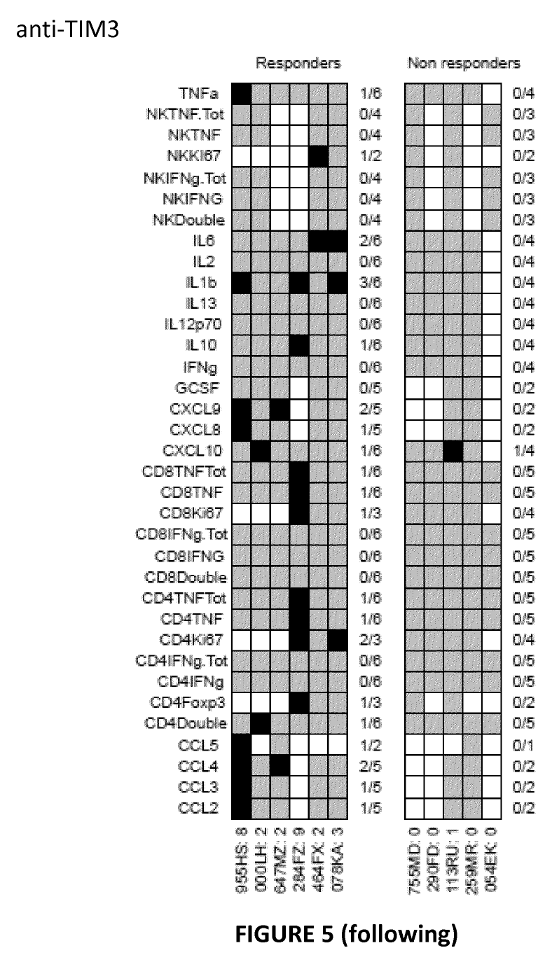

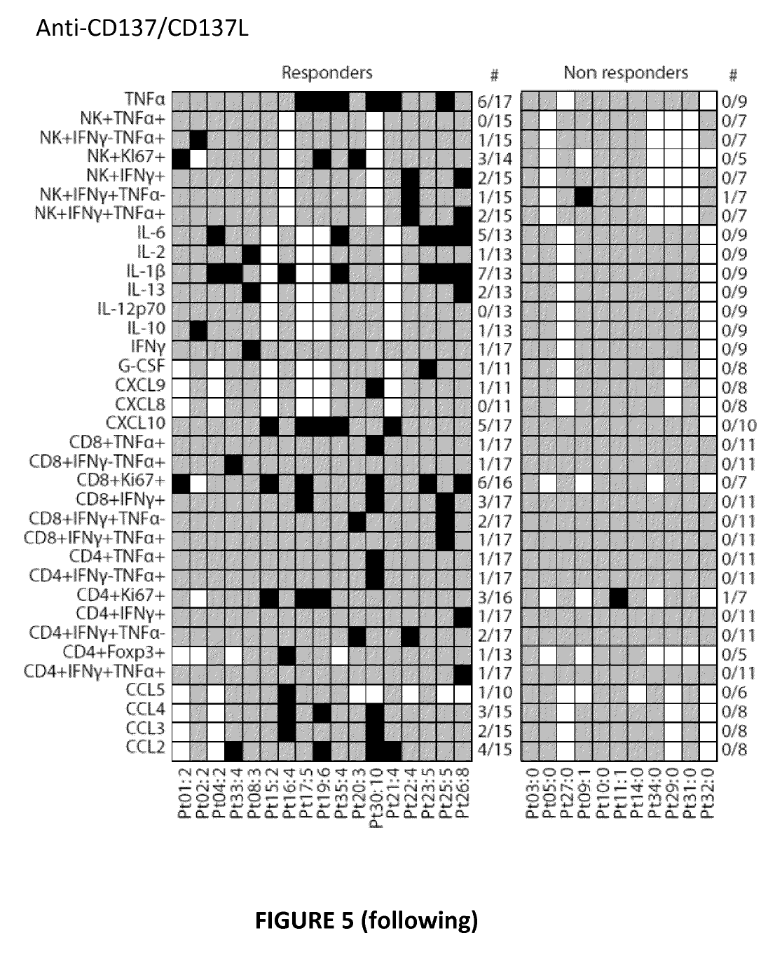

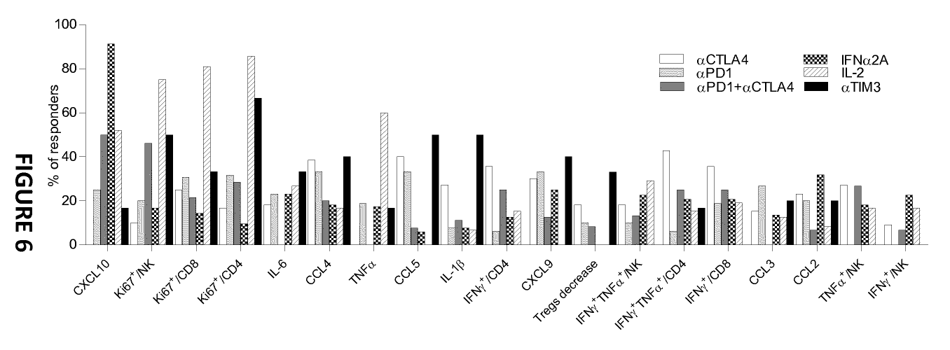

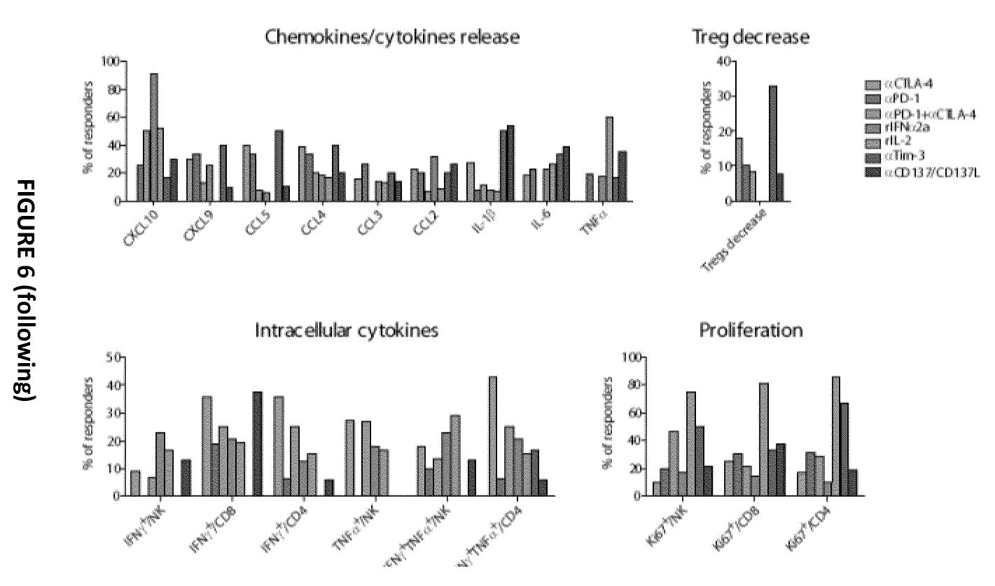

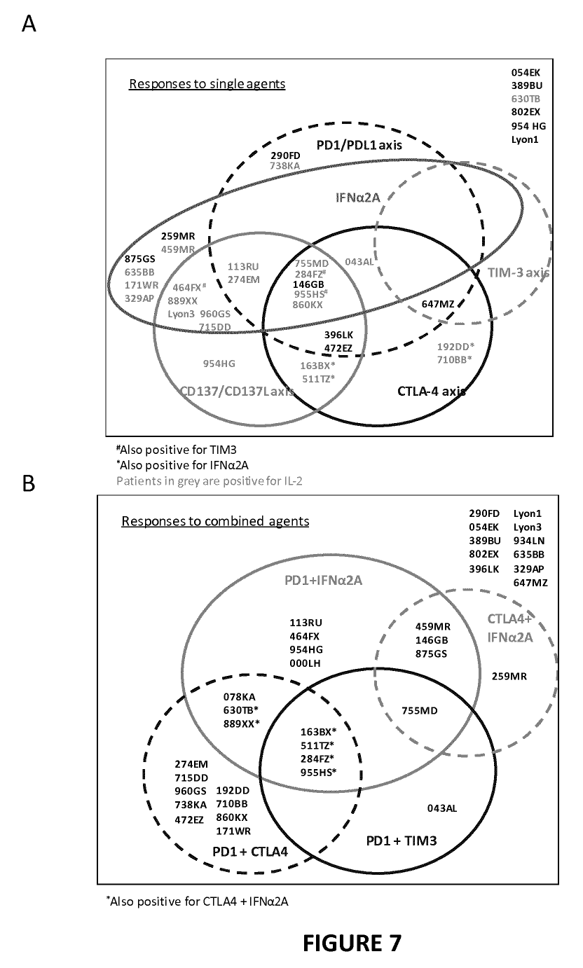

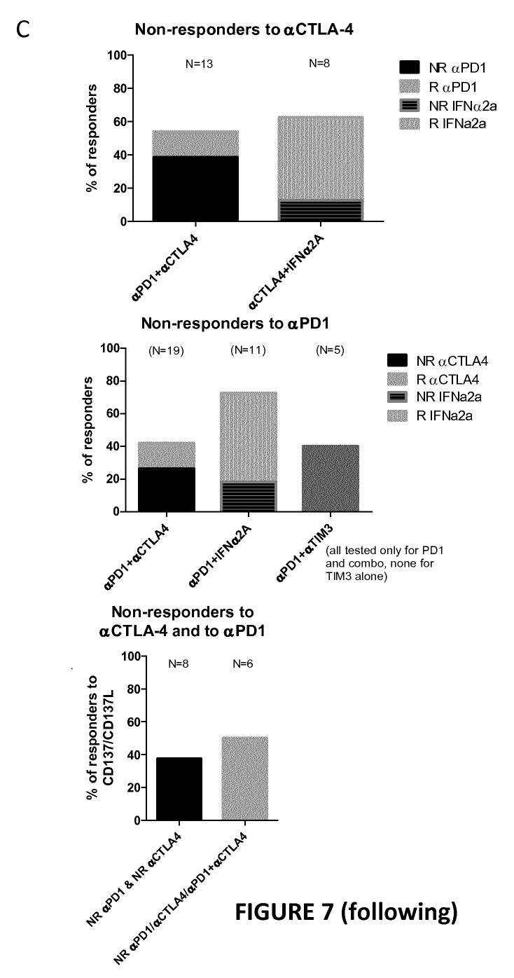

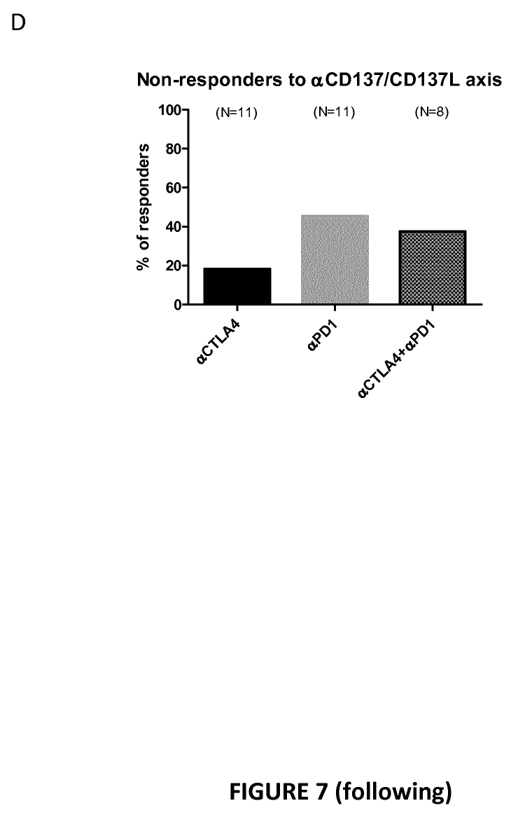

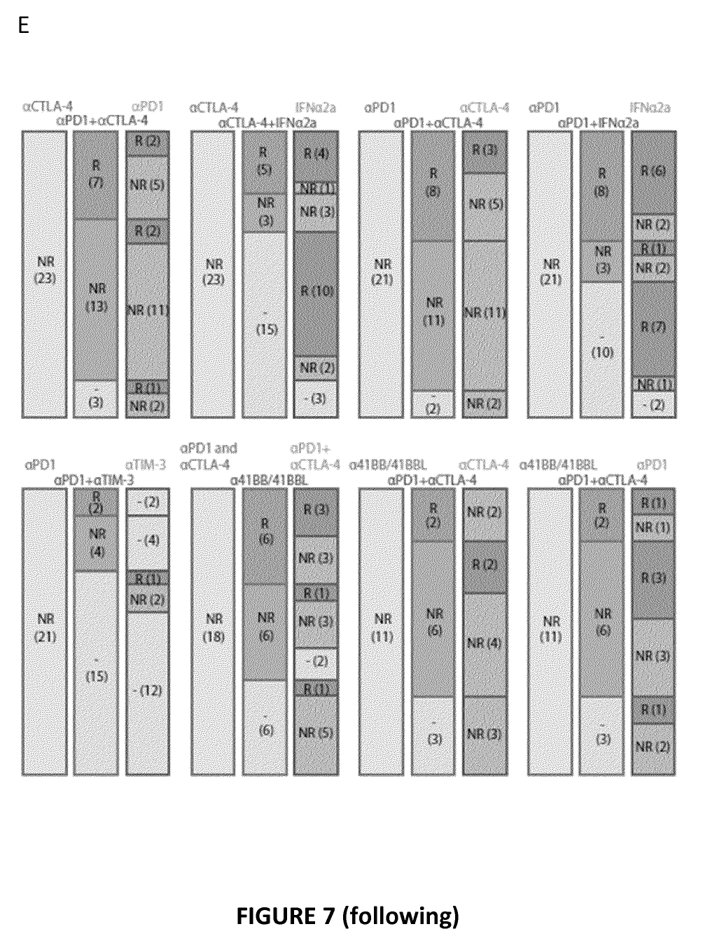

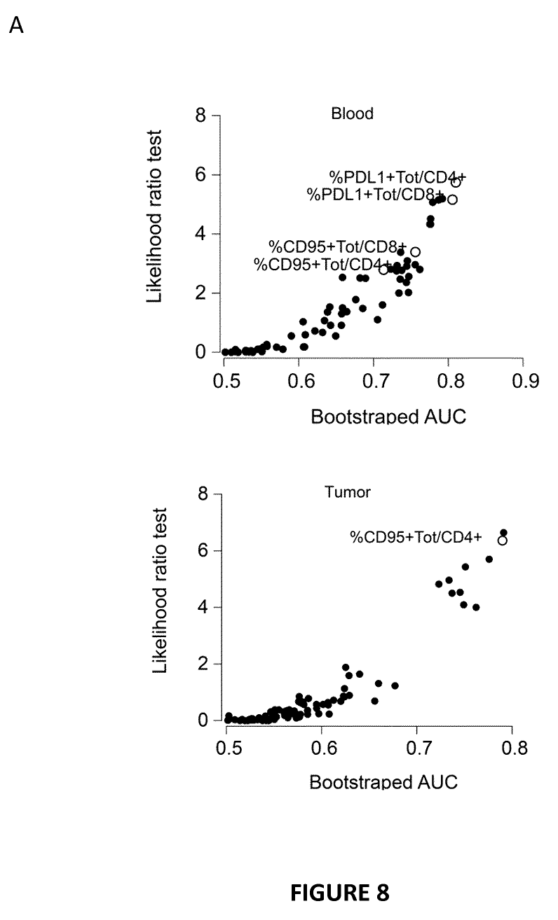

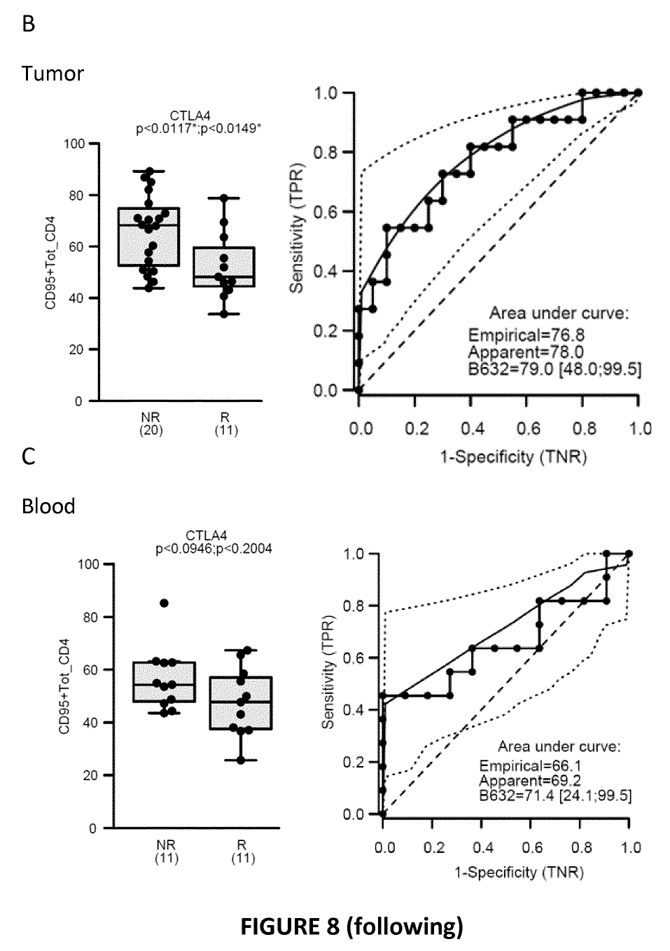

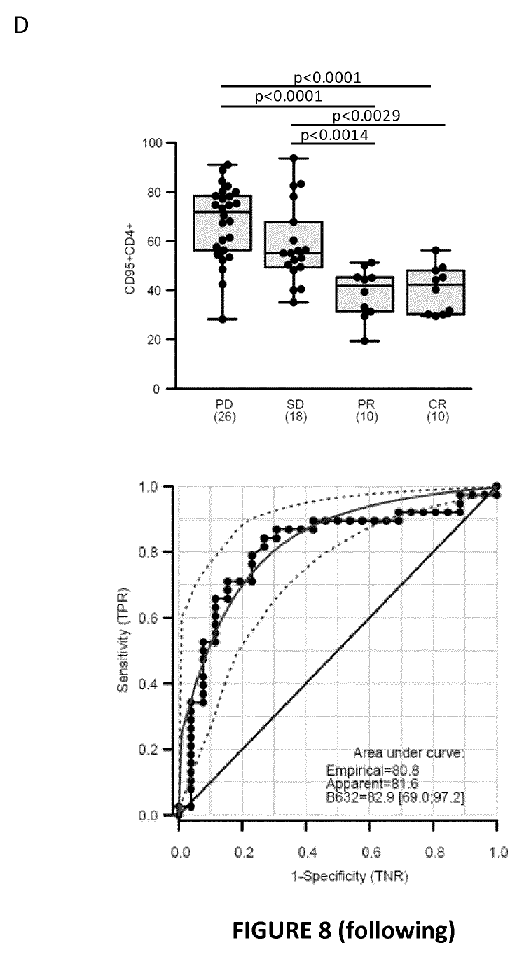

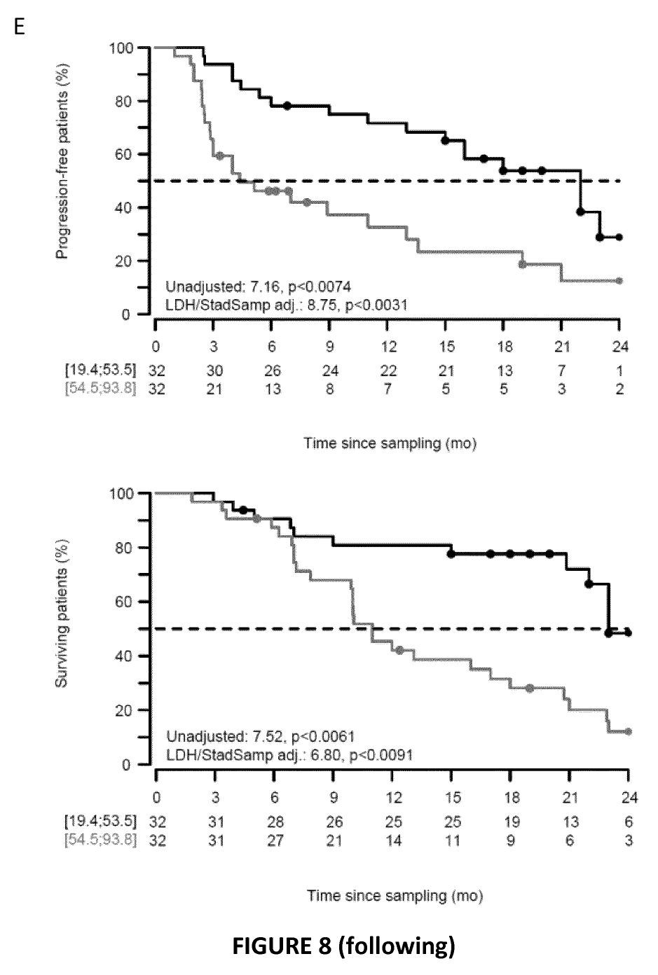

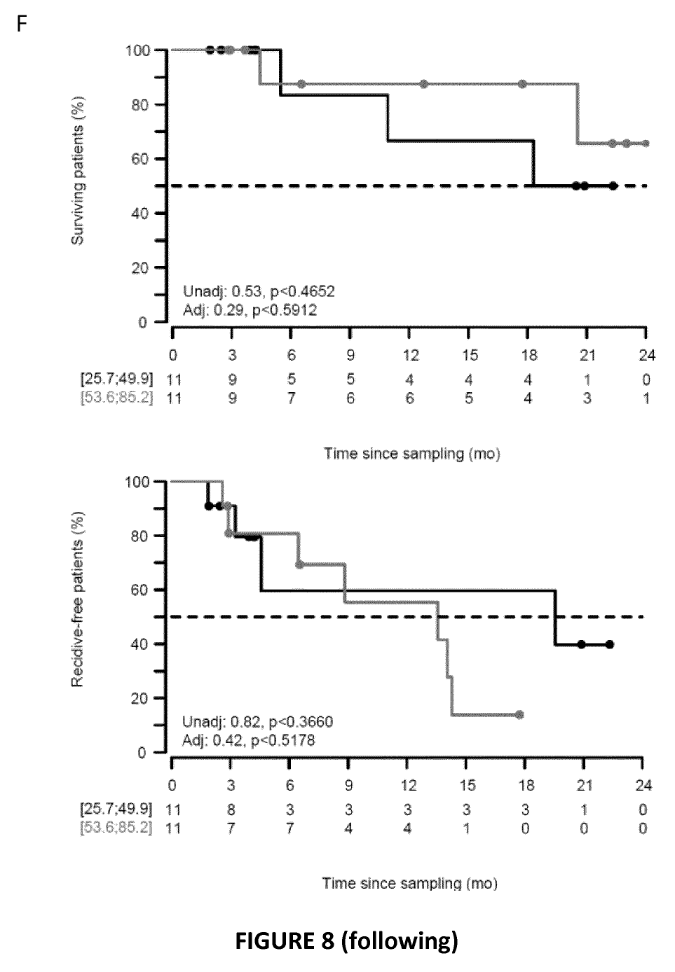

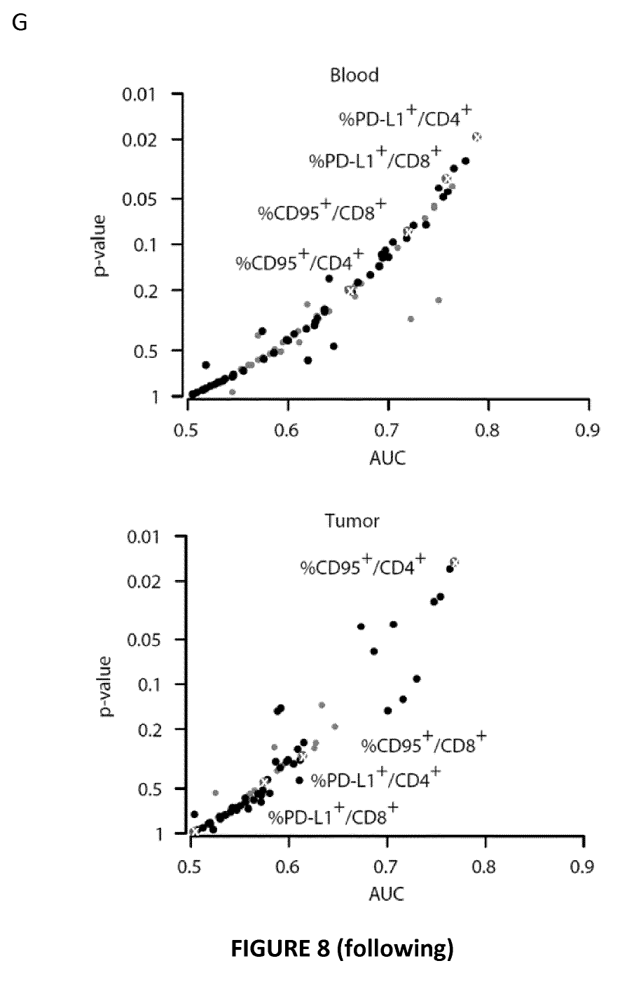

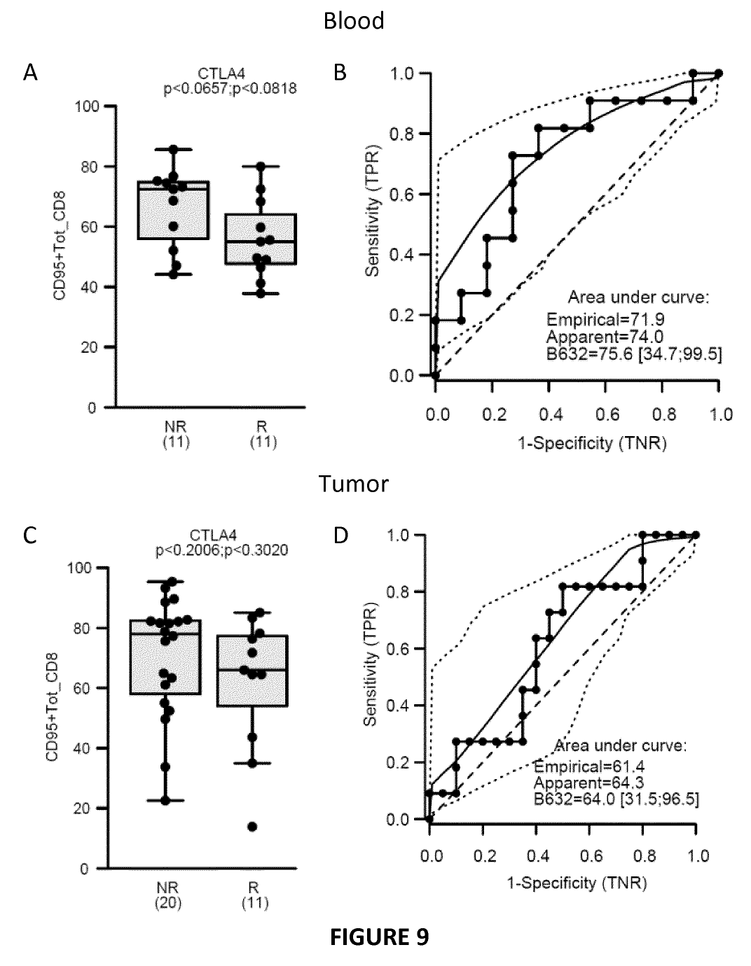

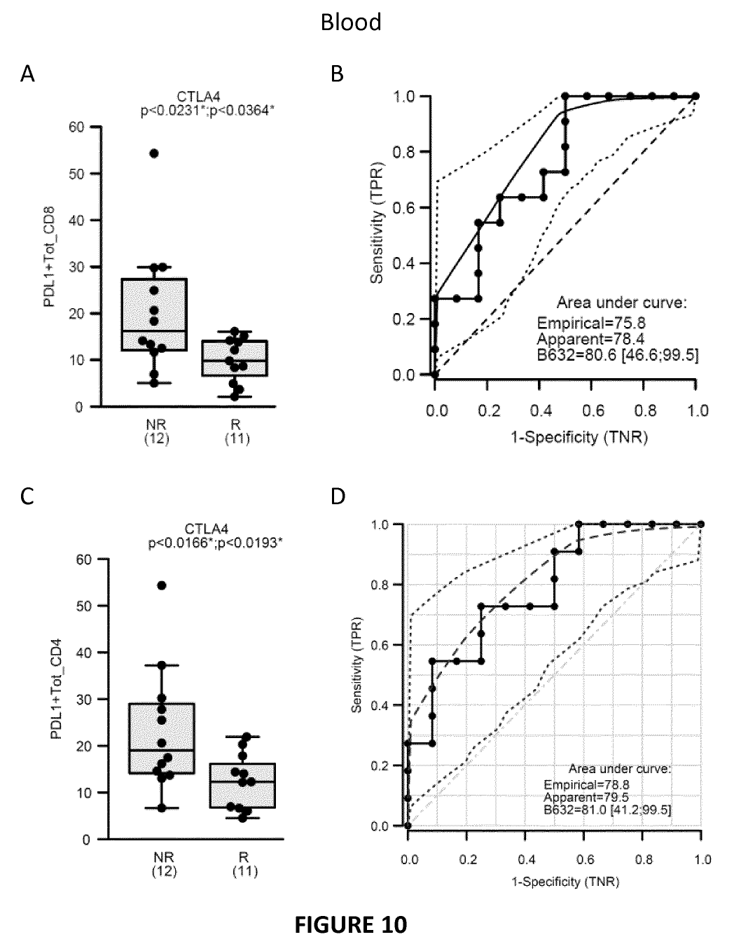

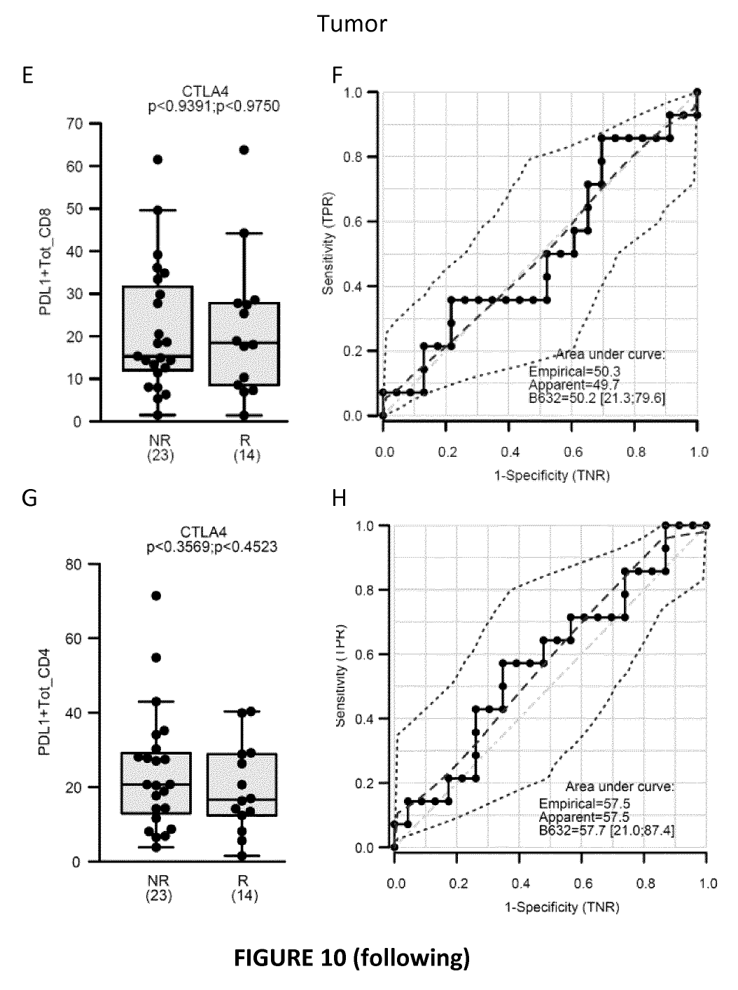

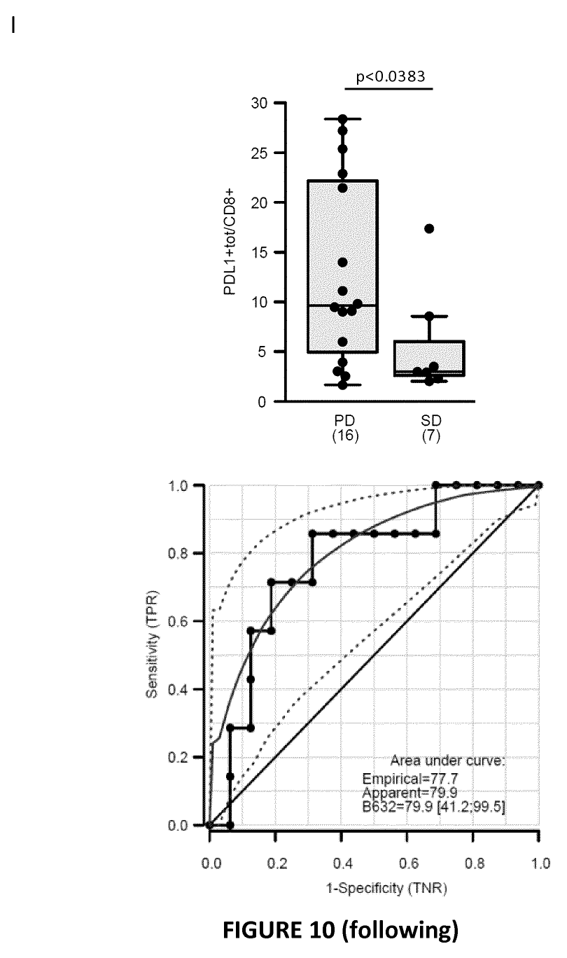

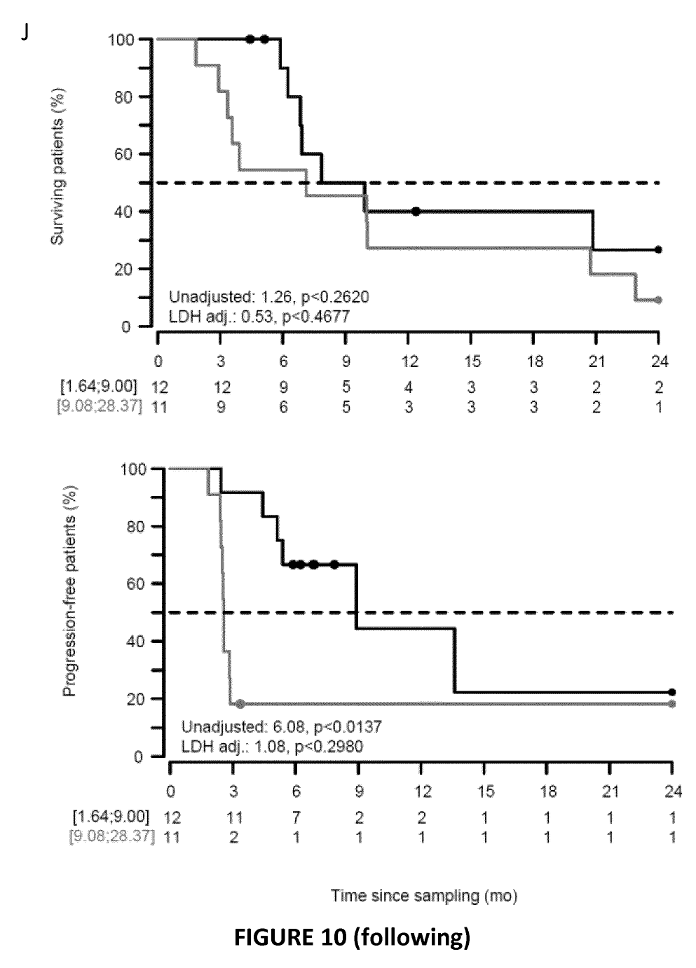

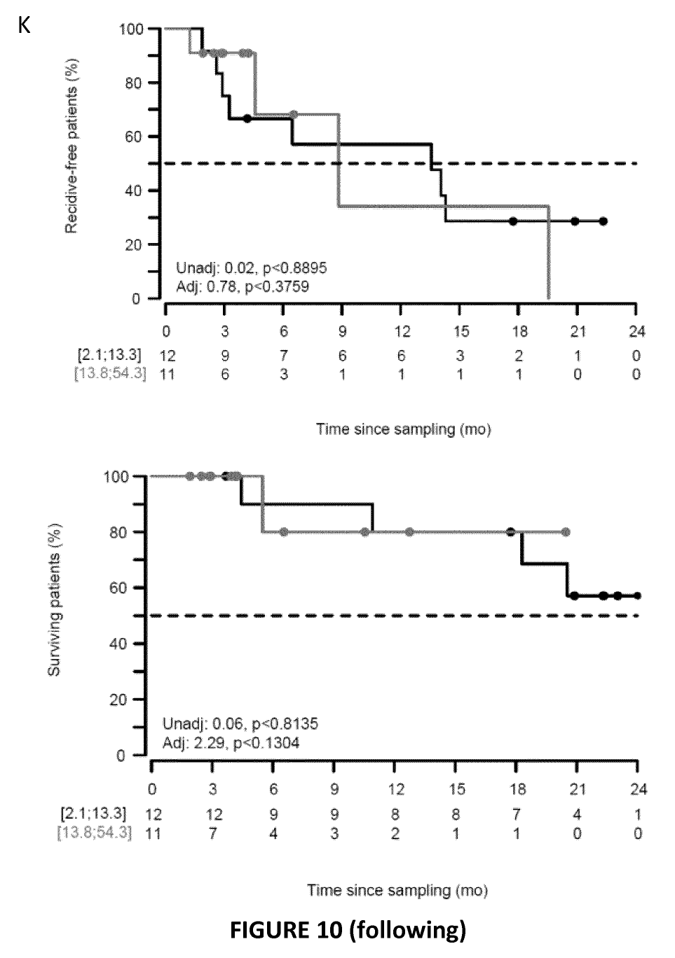

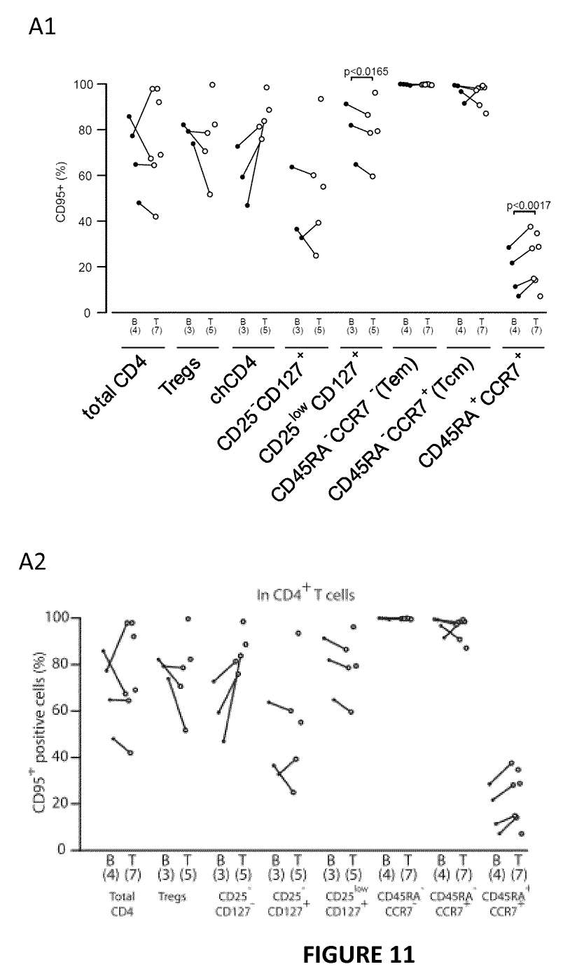

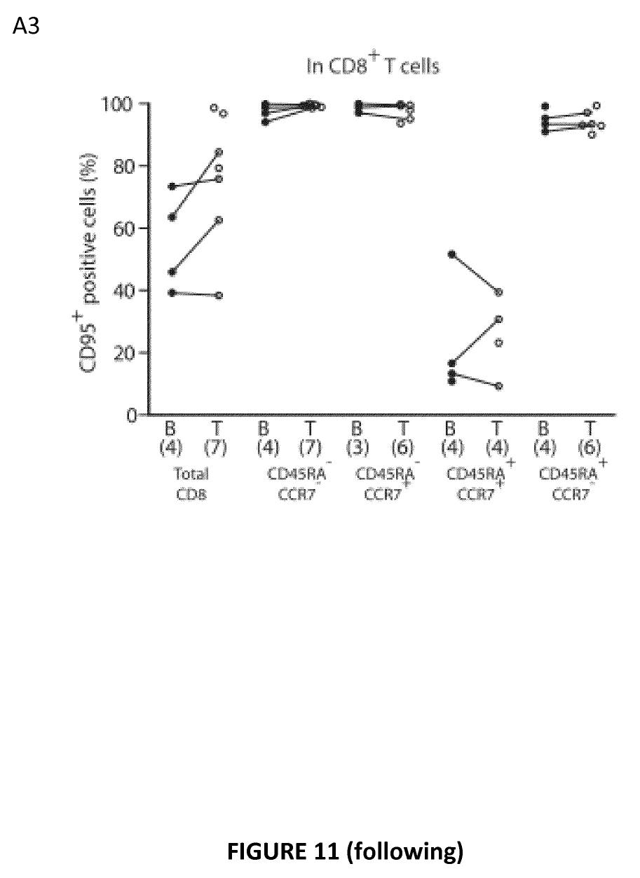

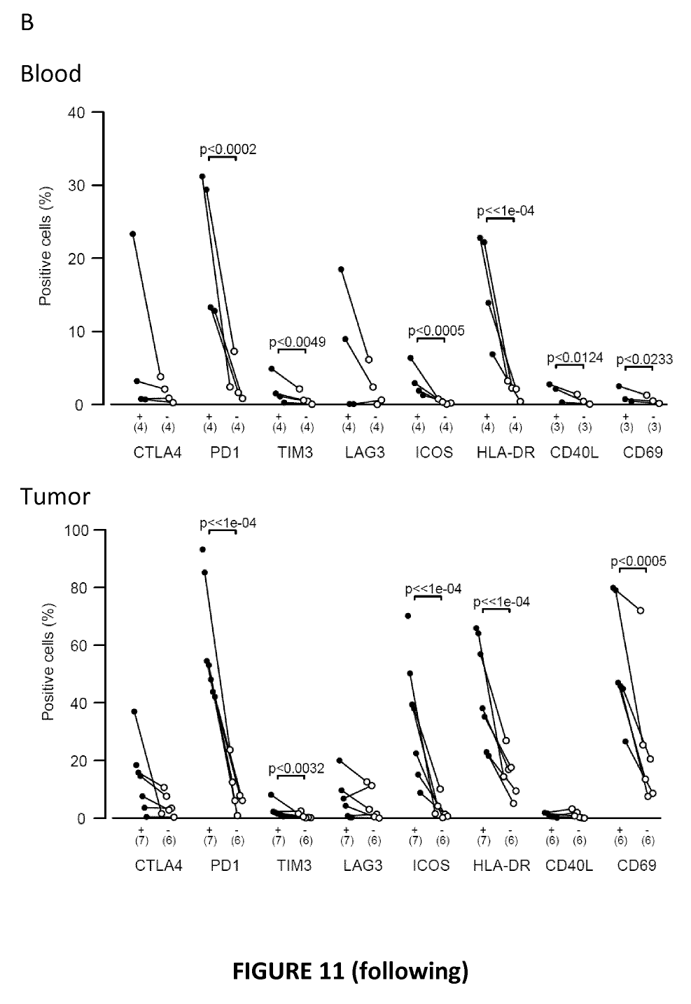

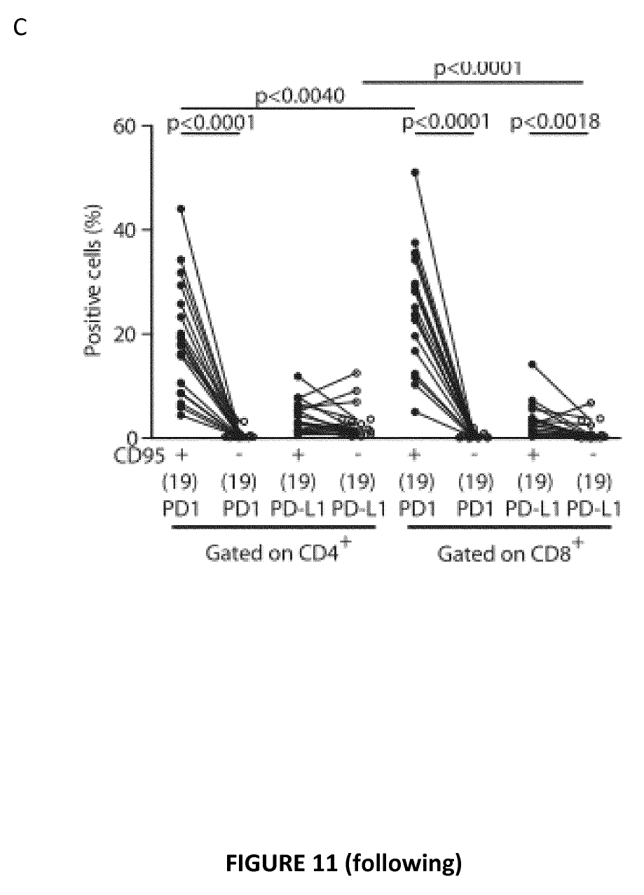

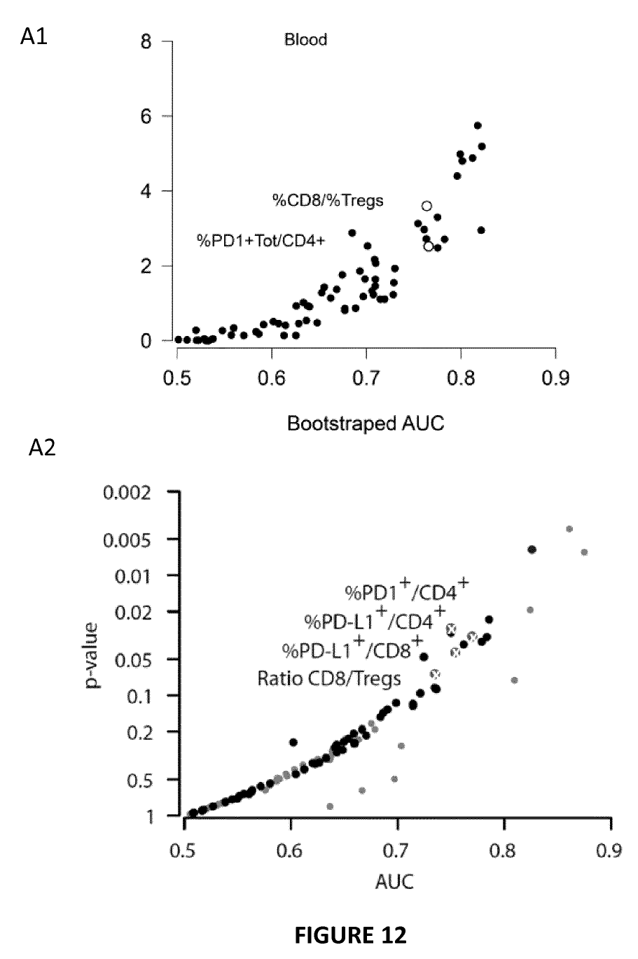

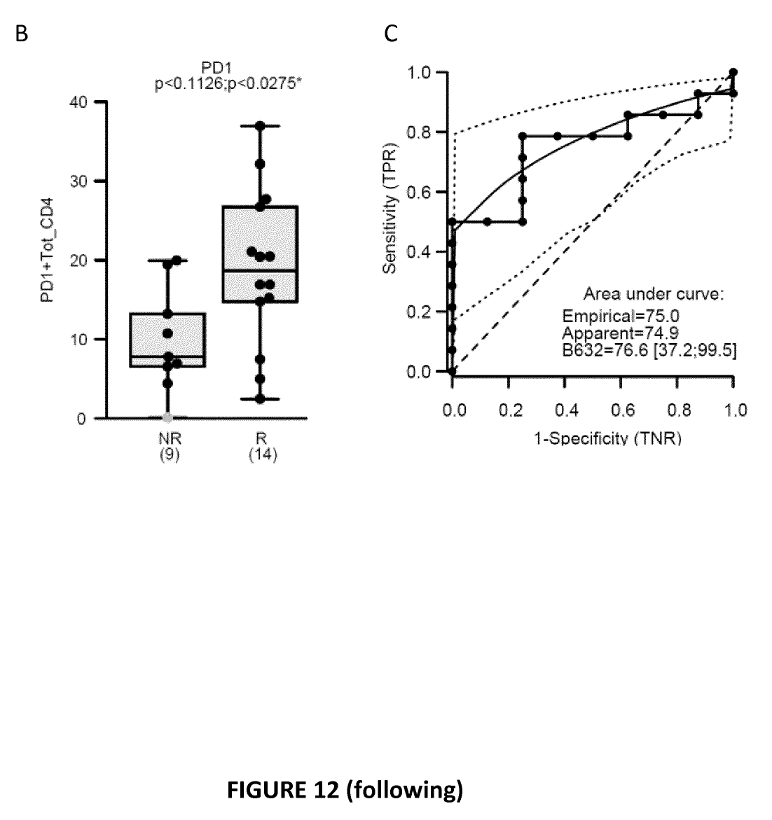

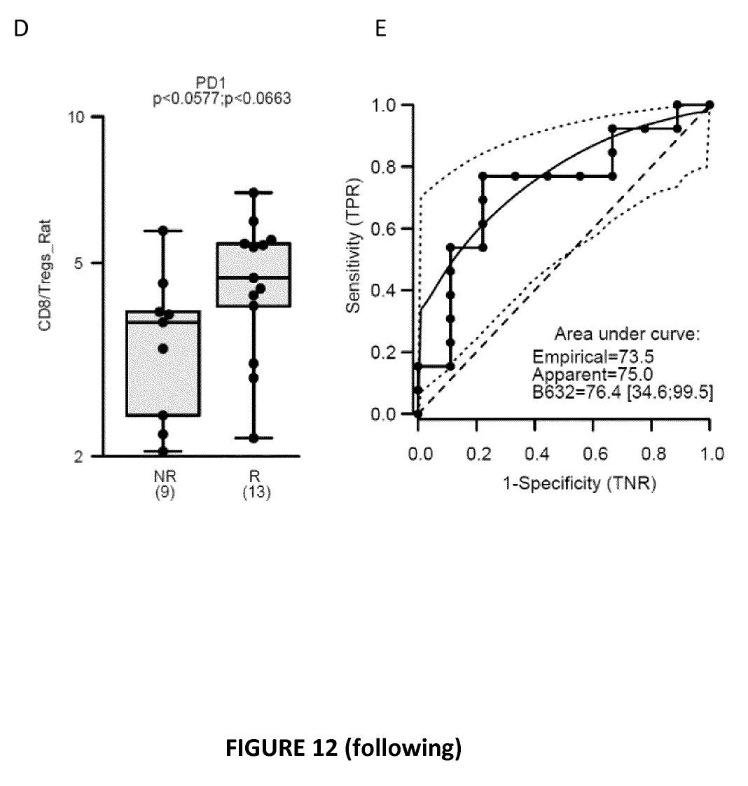

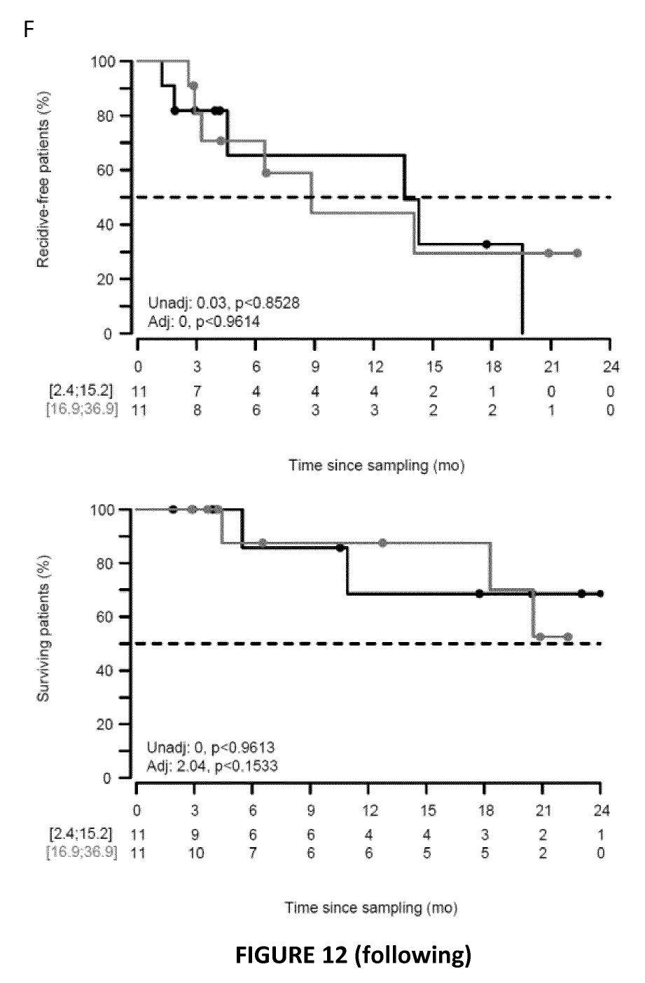

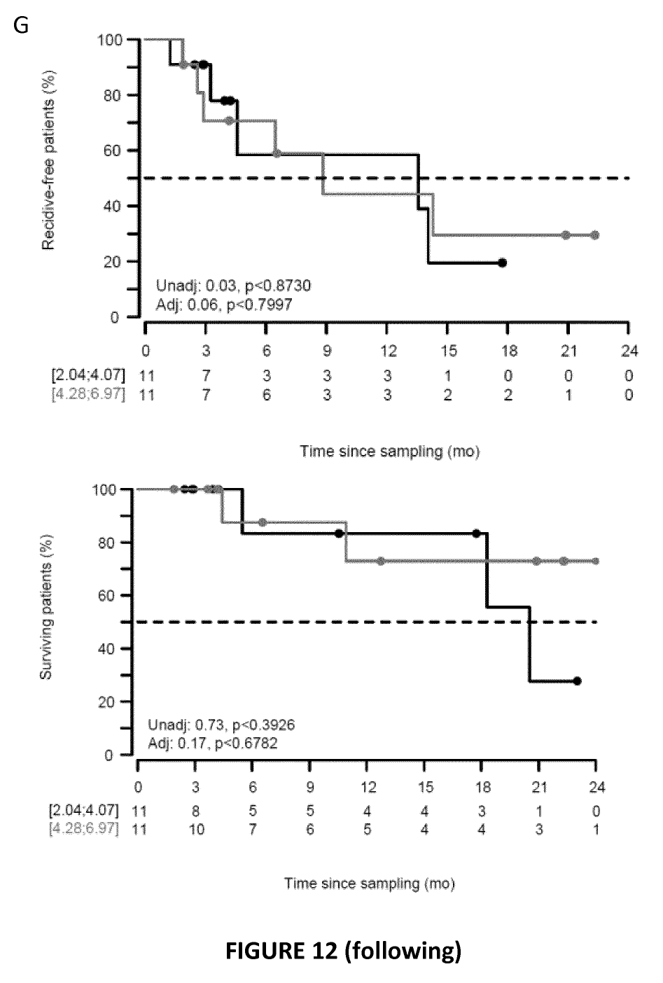

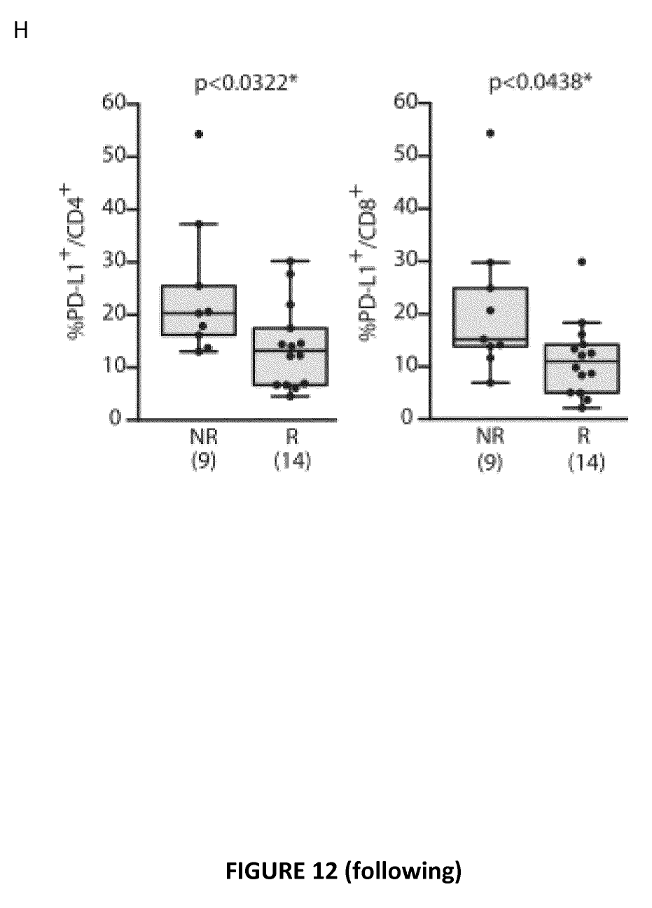

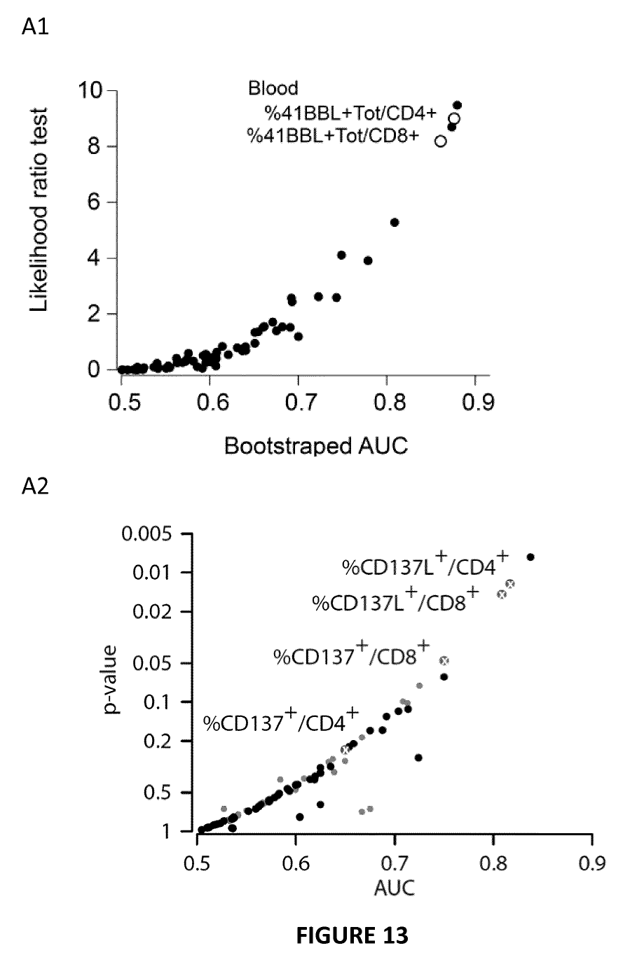

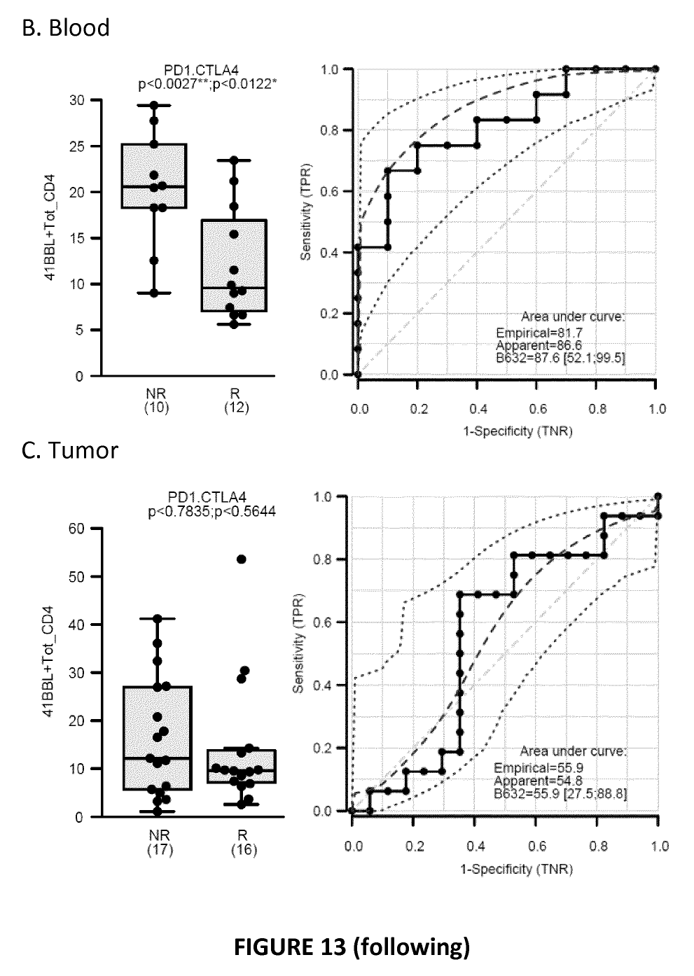

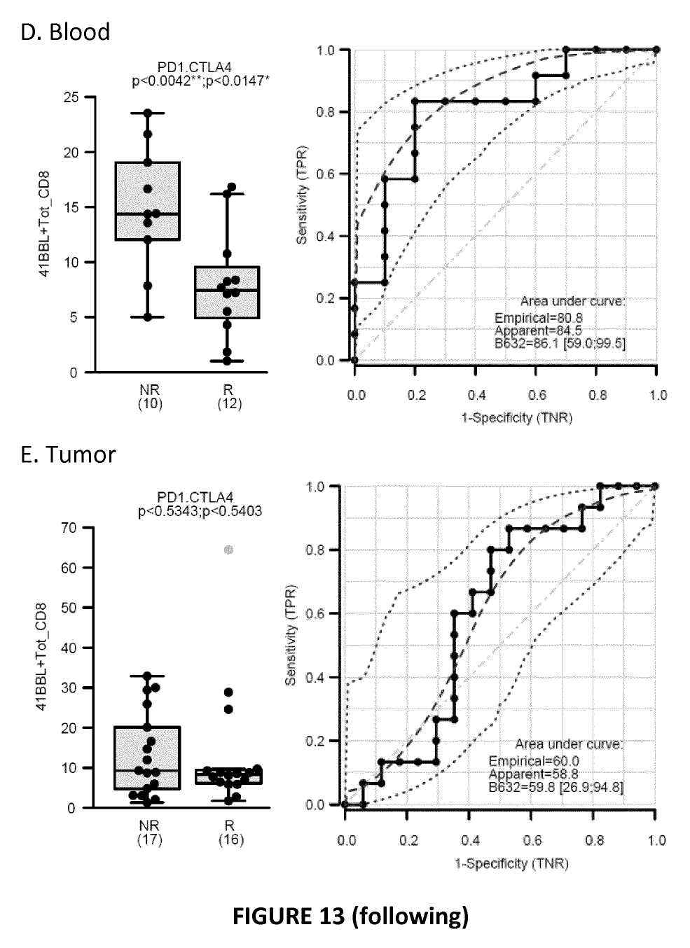

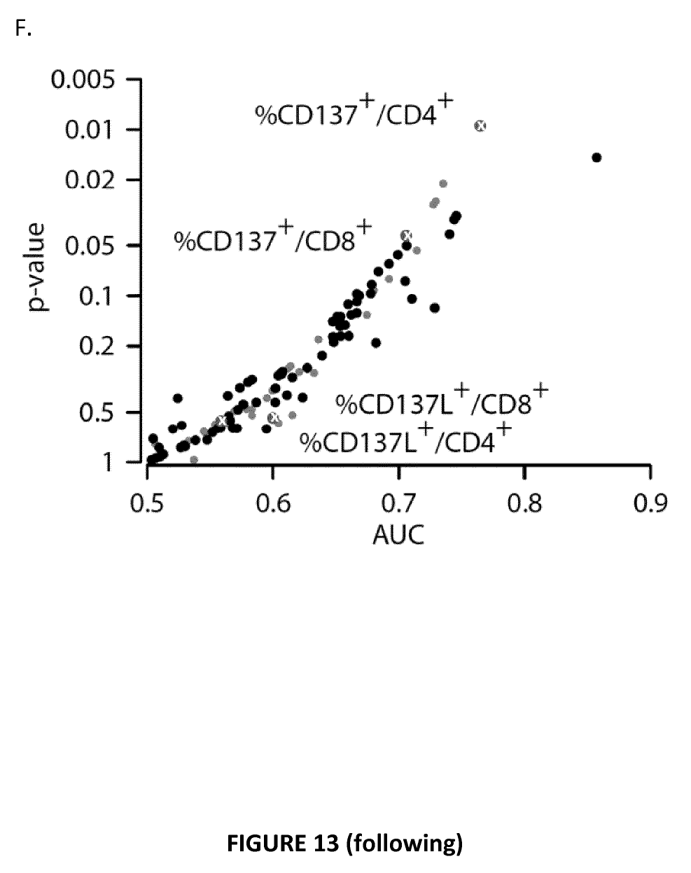

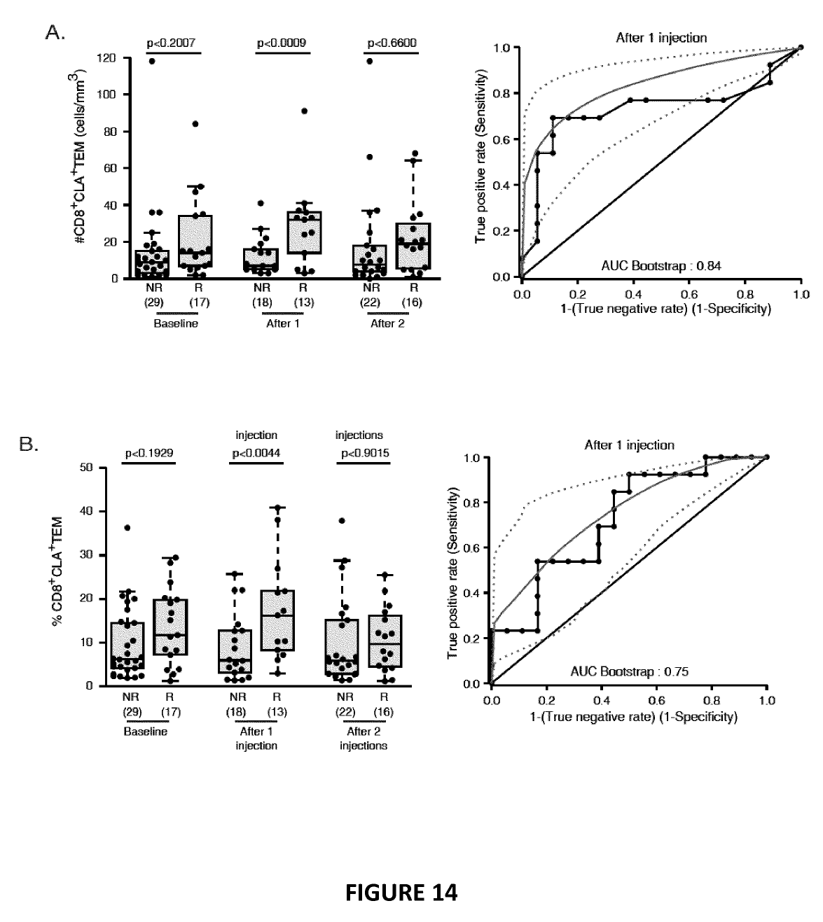

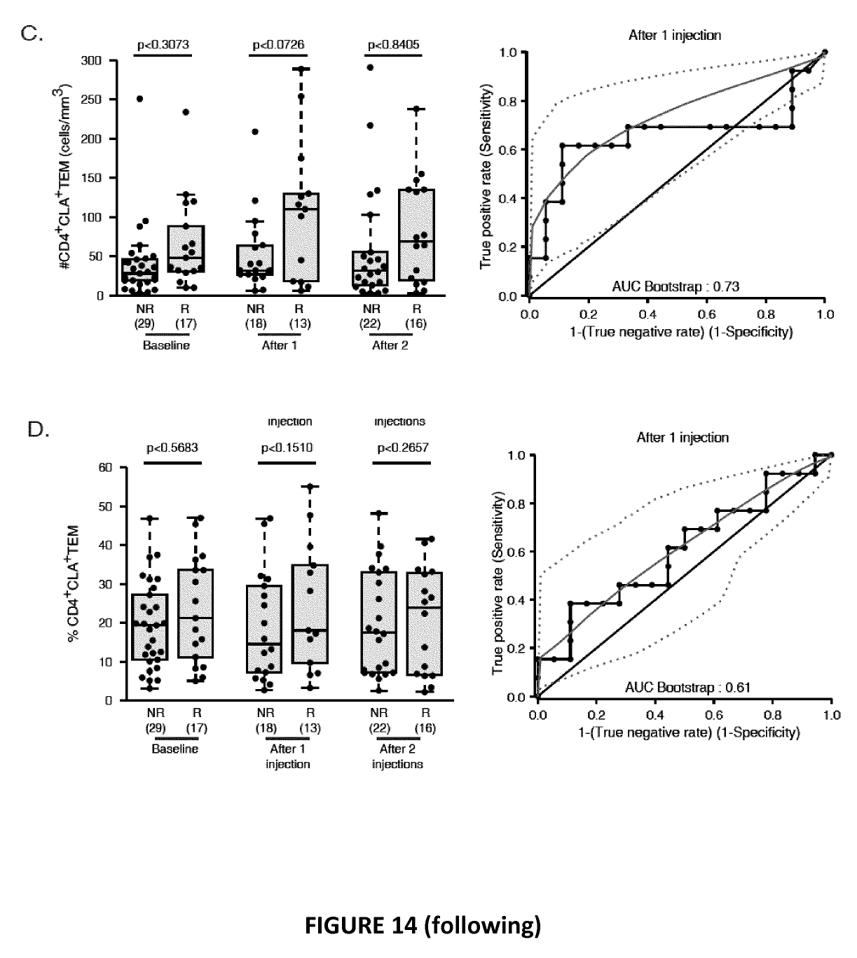

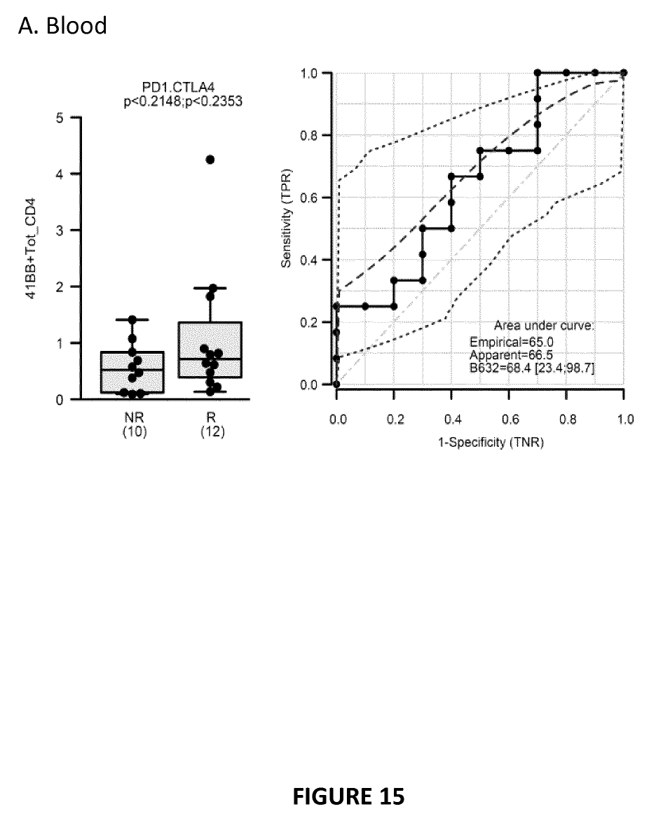

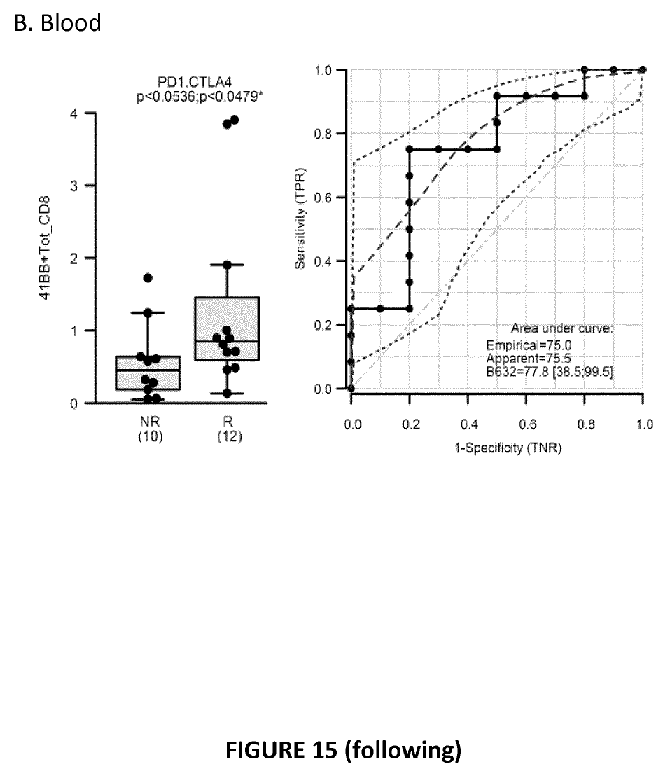

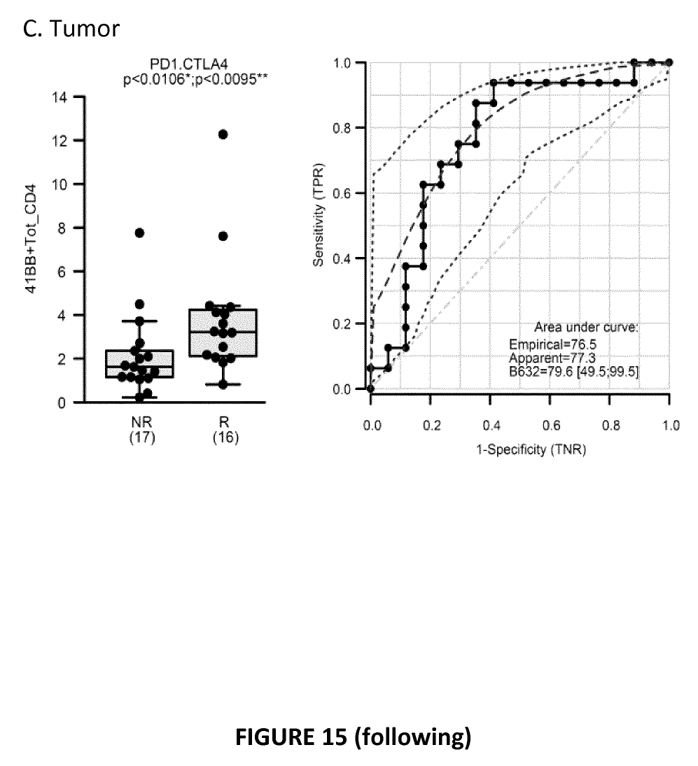

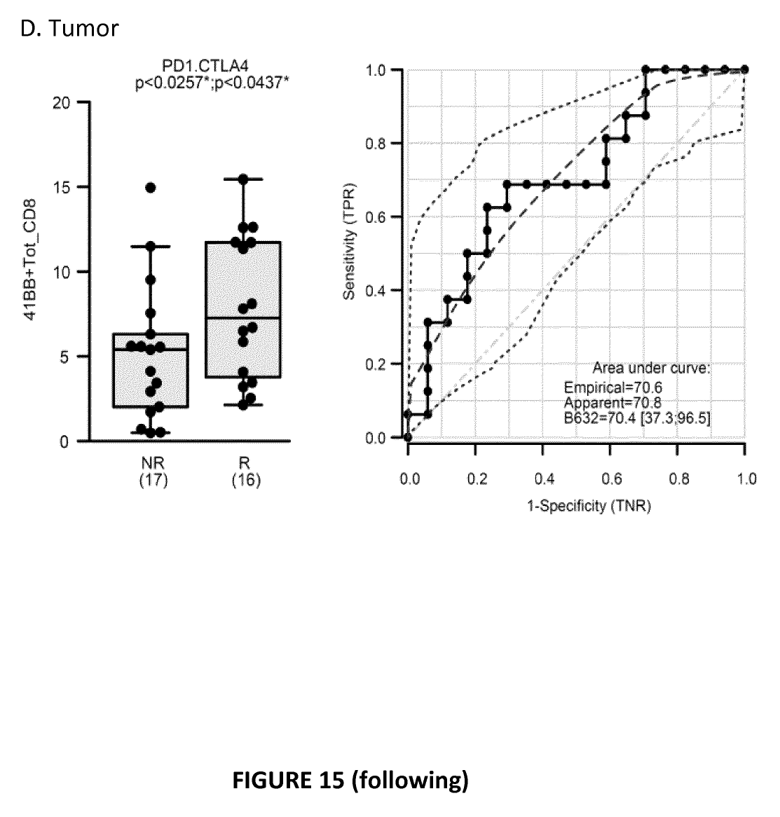

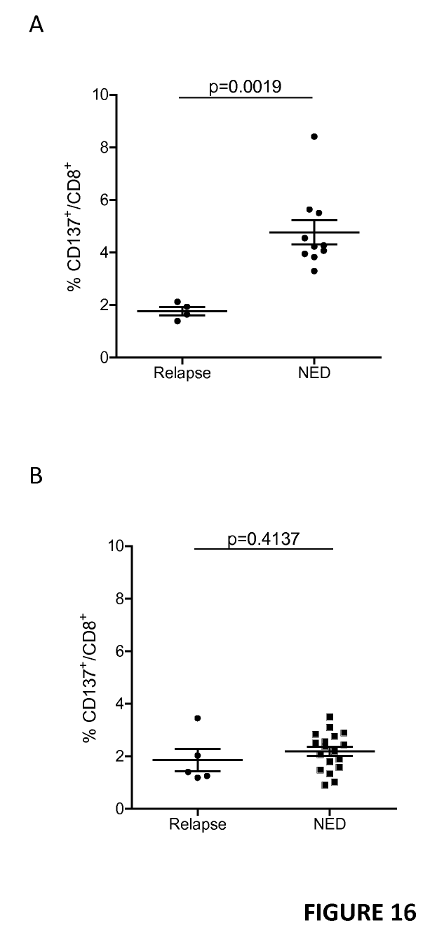

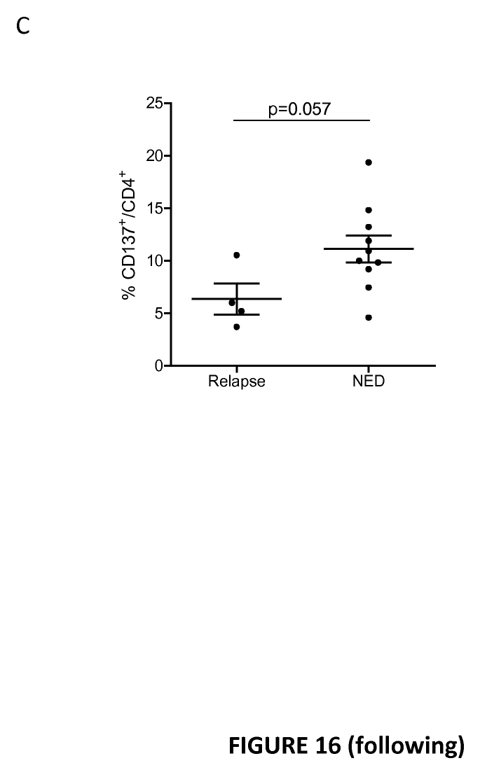

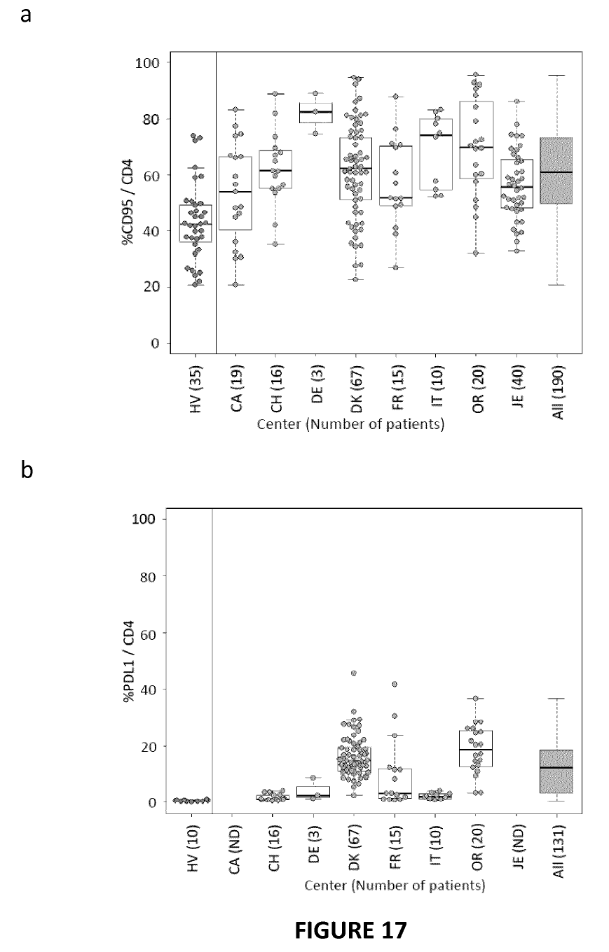

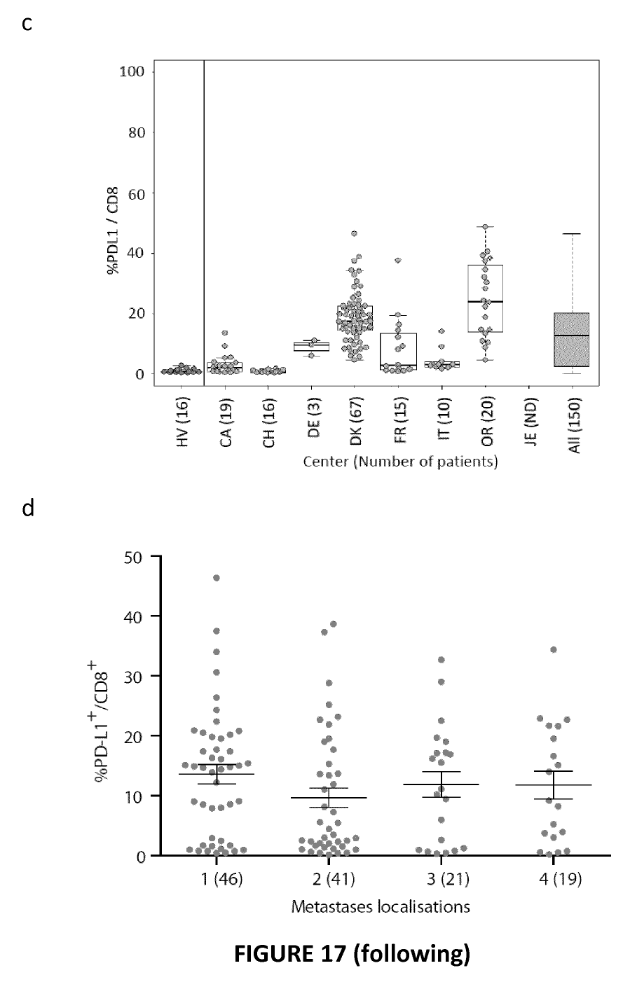

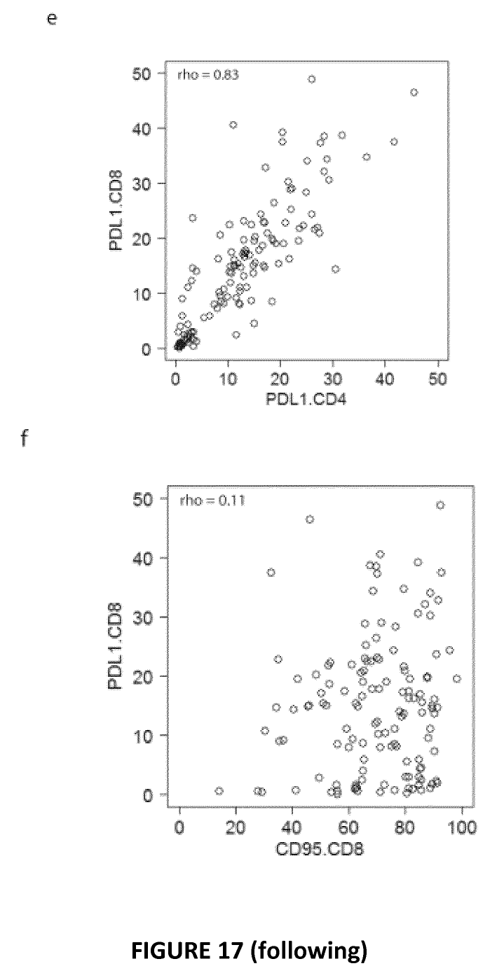

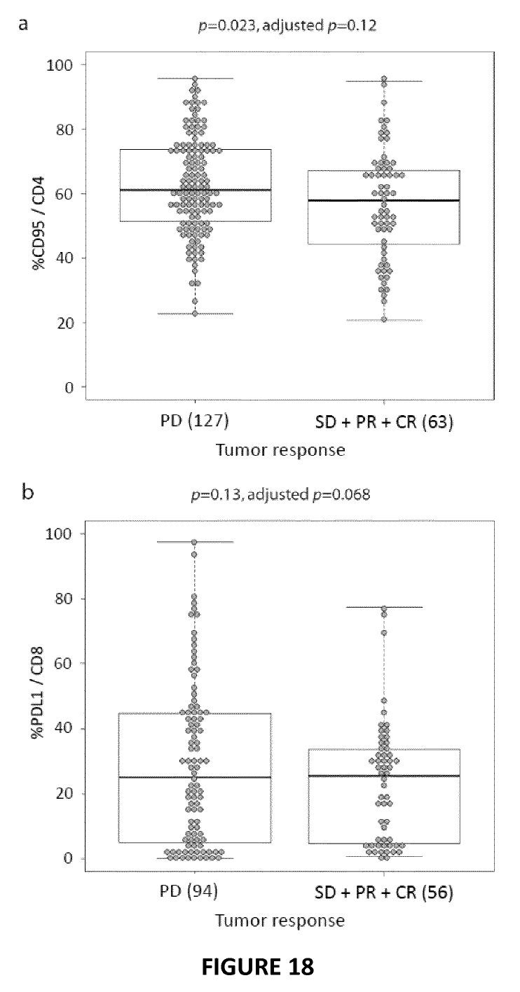

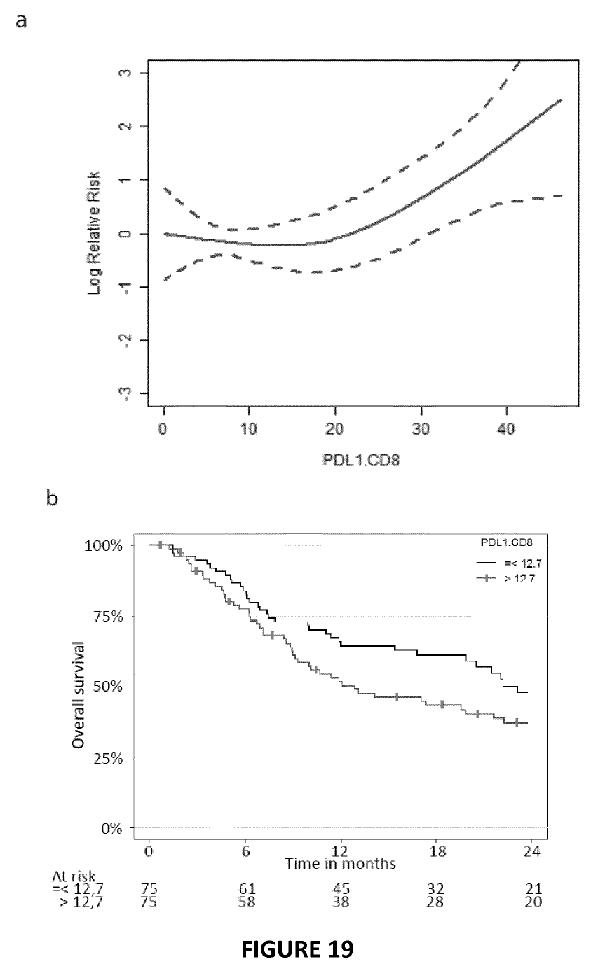

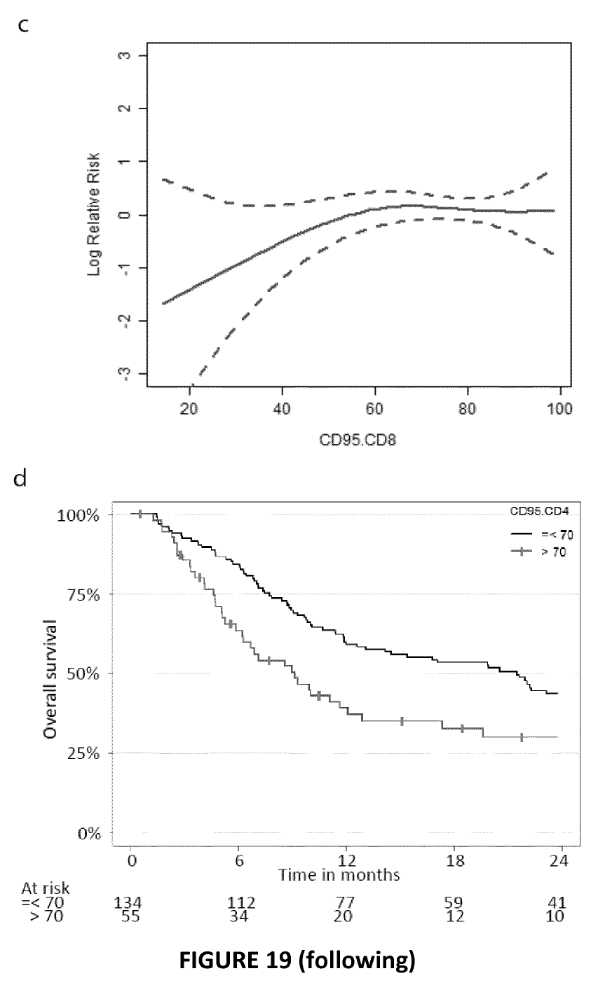

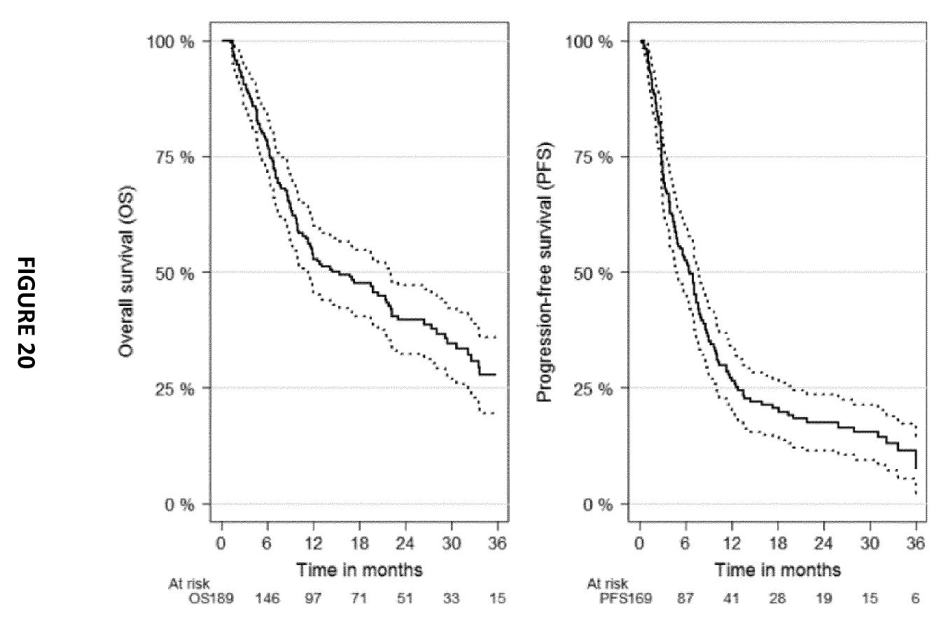

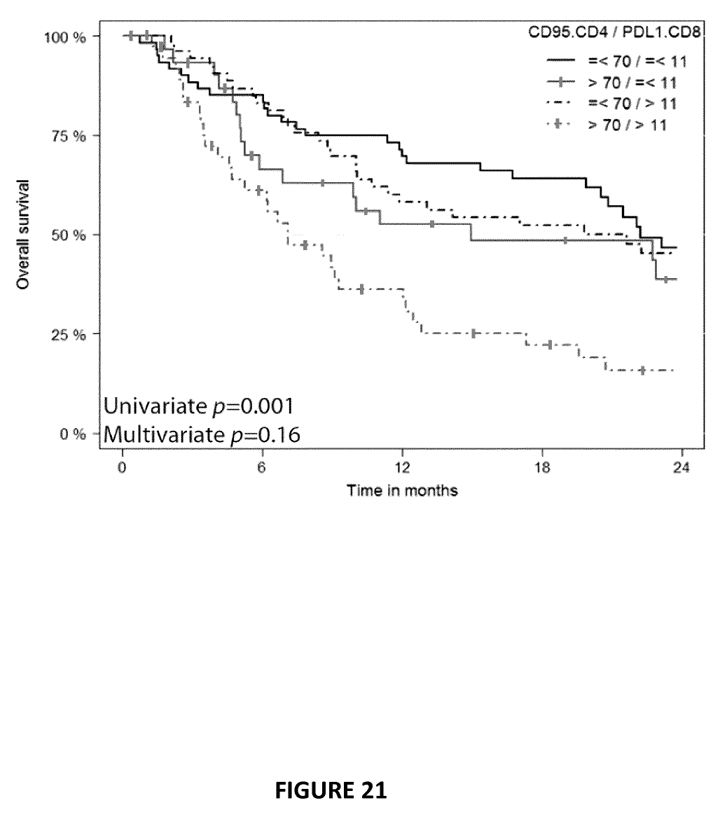

Other characteristics and advantages of the invention are given in the following experimental section (with reference to FIGS. 1 to 21), which should be regarded as illustrative and not limiting the scope of the present application.

### EXPERIMENTAL PART

### Example 1—Personalized Immuno-Oncology and Predictors of Responses to Immune Check-Point Blockade in Stage III Melanoma

### Results

### Functional Assays on Ex Vivo Dissociated mLN Using One or Several Therapeutic mAb.

The patients' population consisting in stage III MM (Metastatic Melanoma, also herein identified as “MMel”) benefiting from a surgery for a metastatic lymph node has been previously described (Jacquelot et al JID in press). Briefly, one third presented more than 3 invaded LN (Lymph node), 52% were ulcerated MM, exhibiting in 55% cases a mutated B-RAF oncogene, in >30% cases a dysthyroidism and undergoing an adjuvant therapy in >50% cases. After mechanical and enzymatic digestion of the metastatic draining lymph node (MLN) of MM, CD45⁻ tumor cells represented 4-98±4.8% SEM of whole cells and tumor composition was analyzed by flow cytometry on live cells in 39 specimen paired with blood. Based on a comprehensive immunophenotyping of 252 parameters per patient featuring cellular types, activation status, naïve or memory phenotypes and activating or inhibitory receptors or ligands in paired blood and MLN performed in 39 MM, inventors found that blood markers were as contributive as tumor-associated (TIL) immunotypes, and parameters associated with lymphocyte exhaustion/suppression showed higher clinical significance than those related to activation or lineage (Jacquelot et al JID in press). Inventors previously reported that CD45RA⁺CD4⁺ and CD3⁻CD56⁻ TILs appear to be independent prognostic factors of short progression-free survival (PFS) while high NKG2D expression on CD8⁺ TILs and low Treg TILs were retained in the multivariate Cox analysis model to predict prolonged overall survival (OS).

The next step consisted in analyzing the dynamics of these parameters after incubation with monoclonal antibodies (mAb)+/−cytokines on 37 patients. Dissociated mLN were incubated for 18 h and up to 5 days in 15 conditions of stimulation aimed at assessing the reactivity of various subsets of CD4⁺, CD8⁺, CD25⁺CD127⁻ T cells, NK, CD3⁻CD56⁻, CD45⁻ cells to mAb targeting four functional axis (PD-1/PD-L1, CTLA-4, CD137/CD137L, TIM3), cytokines (IFNα2a (ROF), IL-2) and their combinations (PD-1+ROF, CTLA-4+ROF, PD-1+TIM3, PD-1+CTLA-4) (FIG. 1 and Table 1).

TABLE 1
Reagents used for stimulation assay in-vitro

| Stimulation | | | Final | |
|---|---|---|---|---|
| Condition | Clone | Source | Concentration | |
| Medium control | | | | |
| mIgG1 | 11711 | R&D | 10 | μg/ml |
| anti-PD1 | PD1m.3 | Dr. Chen's lab | 10 | μg/ml |
| anti-PDL1 | 5H1 | Dr. Chen's lab | 10 | μg/ml |
| anti-Tim3 | 2E2 | Dr. Anderson's lab | 10 | μg/ml |
| anti-CTLA4 | BMS-734016 | Yervoy | 10 | μg/ml |
| anti-CD137 | 6B4 | Dr. Choi's lab | 5 | μg/ml |
| anti-CD137L | 5F4 | Dr. Choi's lab | 5 | μg/ml |
| IL-2 | | (Proleukine ®) | 100 | IU/ml |
| | | Norvartis | | |
| IFN-α2A | | (Roferon ®) | 1000 | IU/ml |
| | | Roche Pharma | | |

The immunometrics performed in 48 wells' plate that inventors considered to perform (biological readouts henceforth) were the early (18-24 hrs) Th1 cytokine/chemokine secretory profiles of T and NK cells (monitored in flow cytometric intracellular staining), the cytokine/chemokine accumulation in the 18-24 hrs supernatant (multiplex array and ELISA), the late proliferative response of T cell subsets (flow cytometric Ki67 expression at day 4-5) and the decrease in Treg proportions. Inventors arbitrarily defined “biological responses”, as those exhibiting a >1.5 fold increase over the values obtained with two negative controls (medium and Ig control mAb) in at least two independent biological readouts (out of n=40 immunological assays, 35 were used with a threshold at 95% of detected values) for each of the 15 culture conditions, except for CD4⁺FoxP3⁺ Treg for which they retained a >1.5 fold decrease compared with the baseline levels in responders over non-responders. The inter-individual variability for specimen manipulation and flow cytometry was minimal, as exemplified for 2 specimens (FIG. 2). The overall charts summarizing the relative levels of response in responders and non-responders for each biological readout in each culture conditions are presented in FIGS. 3-5. As a positive control, IL-2 stimulation of mLN frequently induced of T and NK cell proliferation as well as cytokine release mostly by NK cells and late Treg accumulation in 60% cases (FIG. 6 and FIG. 3). Additionally, ex vivo stimulation with rIFNα2a led to high Cxcl10 chemokine release (in 25/28 cases) (FIG. 3). mLN responding to PD-1 blockade exhibited the following traits: NK cell proliferation in 20% cases, CD4⁺ and CD8⁺ T cell proliferation in 30% cases, TNFα accumulation in 18.75% mLN while CCL3, CCL4, CCL5, Cxcl9 and Cxcl10 were released in more than 25% cases (FIG. 6 and FIG. 4). mLN responding to CTLA-4 blockade translated into these dynamic traits: CD8⁺ Ki67⁺ in 25% cases, CD4⁺ and CD8⁺ IFNγ intracellular staining in 35-40% cases, Cxcl9 detectable in 30% cases while CCL4 was released in 38% cases (FIG. 6 and FIG. 4). mLN responding to CTLA-4/PD-1 blockade showed the following hallmarks: NK and CD4⁺ T cell proliferation in 46% and 28% cases respectively, TNFα and IFNγ coexpressing T cells in 25% lesions, and Cxcl10 in 50% cases (FIG. 6, FIG. 5). In contrast, anti-TIM3 Ab led to CD4⁺ T cell proliferation in ⅔ lesions, as well as CCL4 and CCL5 production in ⅖ and ½ cases respectively (FIG. 6, FIG. 5).

The Venn diagrams detailing all the patterns of immune reactivities are depicted in FIG. 7. The proportions of mLN responding to at least one Immuno-Oncology (I-O) axis was around 40%-60%, and >60% for combination mAb (Table 2, FIGS. 7A-B). The proportions of mLN responding to both anti-CTLA-4 and anti-PD-1 mAb separately was 11/37 (29%), among which 45% failed to respond to a concomitant coblockade (Table 2).

TABLE 2
Detailed responses of patients treated with different ICB and cytokines

| | Stimulation in ex vivo mLN assays | | | | | | | | | |
|---|---|---|---|---|---|---|---|---|---|---|
| | | | anti-PD1 + | anti-CD137 | | | anti-PD1 + | | anti- | |
| | anti- | anti- | anti- | and/or | | | anti- | anti- | CTLA4 + | anti-PD1 + |
| Patient | CTLA4 | PD1 | CTLA4 | anti-CD137L | IFNa2a | IL-2 | TIM3 | TIM3 | IFNα2a | IFNα2a |
| 955HS | + | + | + | + | + | + | + | + | + | + |
| 755MD | + | + | − | + | + | + | + | − | + | + |
| 284FZ | + | + | + | + | + | + | + | + | + | + |
| 043AL | + | + | − | − | + | + | + | n/d | − | − |
| 389BU | − | − | − | − | − | − | − | n/d | − | − |
| 511TZ | + | − | + | + | + | + | + | n/d | + | + |
| 000LH | − | + | − | − | + | + | n/d | + | n/d | + |
| 630TB | − | − | + | − | − | + | − | n/d | + | + |
| 459MR | − | − | − | − | + | + | − | n/d | + | + |
| 290FD | − | + | n/d | − | − | − | − | − | − | − |
| 464FX | − | − | − | + | + | + | n/d | + | n/d | + |
| 163BX | + | − | + | + | + | + | + | n/d | + | + |
| 113RU | − | + | − | + | + | + | n/d | − | n/d | + |
| 078KA | + | + | + | + | + | + | n/d | + | n/d | + |
| 889XX | − | − | + | + | + | + | n/d | n/d | n/d | n/d |
| 802EX | − | − | − | − | n/d | + | − | n/d | n/d | n/d |
| 954HG | − | − | − | + | − | + | n/d | n/d | n/d | + |
| 146GB | + | + | − | + | + | − | n/d | n/d | + | + |
| 259MR | − | − | n/d | − | + | − | n/d | − | + | − |
| 054EK | − | − | n/d | − | − | − | n/d | − | − | − |
| 738KA | − | + | + | − | n/d | + | n/d | n/d | n/d | n/d |
| 647MZ | + | + | − | − | n/d | n/d | n/d | + | n/d | n/d |
| 875GS | − | − | − | n/d | + | n/d | n/d | n/d | + | + |
| 472EZ | + | + | + | + | n/d | n/d | n/d | n/d | n/d | n/d |
| 396LK | + | + | − | + | n/d | n/d | n/d | n/d | n/d | n/d |
| 960GS | − | − | + | + | + | + | n/d | n/d | + | + |
| LYON3 | − | − | − | + | + | + | n/d | n/d | n/d | n/d |
| 715DD | − | − | + | + | + | + | n/d | n/d | n/d | n/d |
| 274EM | − | + | + | + | + | + | n/d | n/d | n/d | n/d |
| 171WR | − | − | + | n/d | + | + | n/d | n/d | n/d | n/d |
| 860KX | + | + | + | n/d | + | + | n/d | n/d | n/d | n/d |
| 635BB | − | − | − | n/d | + | + | n/d | n/d | n/d | n/d |
| 710BB | + | − | + | n/d | + | + | n/d | n/d | n/d | n/d |
| LYON1 | − | − | − | n/d | − | − | n/d | n/d | n/d | n/d |
| 329AP | − | − | − | n/d | + | + | n/d | n/d | n/d | n/d |
| 192DD | + | + | + | n/d | + | + | n/d | n/d | n/d | n/d |
| 934LN | − | − | − | n/d | n/d | n/d | n/d | n/d | n/d | n/d |
| Total | 14/23 | 16/21 | 16/18 | 17/11 | 25/6 | 26/6 | 6/5 | 6/5 | 11/4 | 15/5 |
| R/NR | (37.8) | (43.2) | (47.1) | (60.7) | (80.6) | (81.2) | (54.5) | (54.5) | (73.3) | (75) |
| (% of R) | | | | | | | | | | |

60% mLN ( 17/28) responded to agonistic anti-CD137/anti-CD137L Ab, among which 35% ( 6/17) failed to respond to any of the classical ICB (anti-CTLA-4 or anti-PD-1 Ab) (Table 2). Among the responders to anti-CD137 Ab, about 64% responded to either CTLA-4 or PD-1 blockade (Table 2). The likelihood of response to any alternate ICB/mAb combinations when failing to respond to anyone of them is depicted in FIG. 7C-D.

In conclusion, inventors' ex vivo mLN assay is a feasible test requiring at least 10 million viable tumoral cells for a diagnosis of prediction of response to 11 various conditions of stimulation, and indicate proportions of responses compatible with the clinical rates.

### Predictive Biomarkers of Response to CTLA-4 Blockade

Given that the ex vivo mLN assay is labor intensive and expansive, inventors addressed whether a predictive biomarker of response to the ex vivo mLN assay could be found in the 779 parameters that they analyzed. Hence, they next performed correlative analyses between paired blood and tumor immunometrics (previously described in Jacquelot et al. JID in press) and ex vivo responses to individual mAb axis for the whole cohort of 37 stage III MM to search for predictive biomarkers of response to ICB or combination therapies in our model system.

The two best immunometrics retained in the model of 779 variables were CD95/Fas and CD274/PD-L1 expression levels on CD4⁺ and CD8⁺ circulating T cells respectively (FIG. 8A and FIG. 9-10,). Indeed, lower expression levels of CD95 by CD4⁺ T cells (and only in blood for CD8⁺ T cells, FIG. 8B-C, FIG. 9) in tumor beds and secondarily in blood at diagnosis were associated with the likelihood to respond in the ex vivo mLN functional assays using anti-CTLA-4 Ab (but not other mAb). The AUC reached a higher predictive value of 0.79 using CD4/CD95 flow cytometric determination in tumors (p<0.014) (FIG. 8B) than in blood (FIG. 8C). A more comprehensive analysis of the phenotyping of T cells and TILs expressing CD95/Fas revealed that this staining markedly increased in tumors compared with blood and in Treg and effector memory as well as central memory CD4⁺ T cells (FIG. 11A). Multicolor flow cytometric staining revealed that circulating CD4⁺CD95⁺ T cells expressed high levels of PD-1, Lag3 and HLA-DR molecules, a phenotype also found in tumor beds where CD4⁺CD95⁺ T cells coexpressed not only PD-1 and CTLA-4 but also some activation markers such as CD69, ICOS and HLA-DR (FIG. 11 B, up and down panels, respectively).

The second predictive immune marker of resistance to CTLA-4 blockade was PD-L1 expression on CD8⁺ and CD4⁺ blood T cells (AUC=0.80, p<0.036 and AUC=0.81, p<0.0193) (FIGS. 10 A-D) while this parameter was not significant in tumor beds (FIGS. 10 E-H).

Based on the ex vivo mLN functional assay and immunometrics obtained in blood and tumors, inventors hypothetized that CD95/Fas and CD274/PD-L1 expression levels on CD4⁺ and CD8⁺ circulating T cells respectively contribute to predict resistance to CTLA-4 blockade in MM.

### Predictive Biomarkers of Response to PD-1 Blockade

As inventors did for CTLA-4 blockade, they next address which immunometrics might be able to best predict ex vivo reactivities to PD-1 blockade. Very few immune parameters were found linked to functional responses to anti-PD-1 Ab. The two best immunometrics retained in the model of >779 variables were the ratio between circulating CD8⁺ lymphocytes and CD127^{low-}CD25^{high}CD4⁺ Treg cells, and PD-1 expression levels on circulating CD4⁺ T cells (FIG. 12). Indeed, higher expression levels of PD-1 (>20%) in circulating CD4⁺ T cells at diagnosis were associated with the likelihood to respond in the ex vivo mLN functional assays using anti-PD-1 Ab (but not other mAb, p<0.028, AUC=0.76) (FIGS. 12 A-B). A CD8/Treg ratio>5 also predicted ex vivo reactivity of mLN to PD-1 blockade (but not to another I-O axis, p<0.067, AUC=0.76) (FIGS. 12 C-D).

Based on the ex vivo mLN functional assay and immunometrics obtained in blood, inventors concluded that the CD8/Treg ratio and PD-1 expression levels on CD4⁺ circulating T cells contribute to predict sensitivity to PD-1 blockade in MM.

### Predictive Biomarkers of Response to CTLA4+PD-1 Co-Blockade

Although mediating impressive clinical benefit in MM (ORR>60% with a PFS>11 months), this combinatorial regimen is also quite synergic for immune related adverse events (>50% grade 3-4 hepatitis or colitis) (20), urging investigators to predict clinical outcome. Interestingly, since the proportions of mLN responding to both anti-CTLA4 and anti-PD-1 Ab separately was 11/37 (29%), among which 45% failed to respond to a concomitant coblockade (Table 2, FIG. 7), inventors hypothetized that the predictive biomarkers of response to concomitant incubation with anti-CTLA-4 and anti-PD-1 Abs would be different immunometrics than the ones described above. Once again, few immune parameters were found associated with functional responses to the coblockade (FIG. 13A). The two best immunometrics retained in the model of 779 variables were the expression levels of CD137L/4-1BBL on circulating CD4⁺ and CD8⁺ lymphocytes (FIGS. 13 B and D). Indeed, detectable expression levels of CD137L in circulating CD4⁺ T lymphocytes at diagnosis were associated with the likelihood to resist to the combination therapy in the ex vivo mLN functional assays concomitantly using anti-PD-1 Ab and anti-CTLA-4 Ab (p<0.012, AUC=0.87 in blood, cut off value: 12.54% (9.01-25.19)). Similarly, detectable expression levels of CD137L in circulating CD8⁺ T lymphocytes at diagnosis were associated with the likelihood to resist to the combination therapy in the ex vivo mLN functional assays concomitantly using anti-PD-1 Ab and anti-CTLA-4 Ab (p<0.015, AUC=0.86 in blood, cut off value: 12.03% (7.86-16.65)). In contrast, high levels of the ligand CD137L/4-1BBL in tumor bed failed to predict resistance to the coblockade in ex vivo mLN functional assays (FIGS. 13 C and E).

The two other best immunometrics retained in the model of 779 variables were the expression levels of CD137/4-1BB on circulating CD4⁺ and CD8⁺ lymphocytes (FIG. 15A-D). Indeed, detectable expression levels of CD137 in blood and tumor CD8⁺ T lymphocytes (and to a lesser extent in CD4⁺ T cells) at diagnosis were associated with the likelihood to respond in the ex vivo mLN functional assays concomitantly using anti-PD1 Ab and anti-CTLA-4 Ab (FIG. 15A-D).

Based on the ex vivo mLN functional assay and immunometrics obtained in blood, inventors concluded that CD137 and/or CD137L expression level(s) on circulating T cells contribute to predict resistance to CTLA-4/PD-1 co-blockade in stage III MM.

### In Vivo Veritas

To verify the potential validity of their immune biomarkers identified originally from correlative matrices between an ex vivo mLN functional assay and blood or tumor immunometrics, inventors retrospectively analyzed their expressions on several cohorts of stage III-IV MM treated with the first line standard immunotherapy ipilimumab (n=64, Table 3).

TABLE 3
Ipilimumab-treated patients characteristics enrolled in four centers

| | | | | | Memorial | |
|---|---|---|---|---|---|---|
| | | | | University | Sloan | University |
| | | All | University | Hospital of | Kettering | of |
| | | patients - | of Stanford - | Siena - N | Cancer Center - | Tubingen - |
| | | N [%] | N [%] | [%] | N [%] | N [%] |
| Gender | M | 37 [57.8] | 21 [52.5] | 6 [60.0] | 7 [63.6] | 3 [100.0] |
| | F | 27 [42.2] | 19 [47.5] | 4 [40.0] | 4 [36.4] | 0 [0.0] |
| Age (yrs)* | | 62.2 [24; 91] | 63.0 [38; 90] | 55.6 [24; 81] | 65 [41; 91] | 64 [53; 74] |
| Stage | III | 34 [53.1] | 33 [82.5] | 1 [10.0] | 0 [0.0] | 0 [0.0] |
| | IV | 30 [46.9] | 7 [17.5] | 9 [90.0] | 11 [100.0] | 3 [100.0] |
| LDH** | Normal | 44 [68.7] | 34 [85.0] | 2 [20.0] | 7 [63.6] | 1 [33.3] |
| | Elevated | 19 [29.7] | 6 [15.0] | 7 [70.0) | 4 [36.4] | 2 [66.7] |
| | NA^{$} | 1 [1.6] | 0 [0.0] | 1 [10.0] | 0 [0.0] | 0 [0.0] |
| Prior | No | 21 [32.8] | 20 [50.0] | 1 [10.0] | 0 [0.0] | 0 [0.0] |
| therapy | Yes | 43 [67.2] | 20 [50.0] | 9 [90.0] | 11 [100.0] | 3 [100.0] |
| Clinical | PD^{$$} | 26 [40.7] | 10 [25.0] | 6 [60.0] | 7 [63.6] | 3 [100.0] |
| response | SD^{$$} | 18 [28.1] | 10 [25.0] | 4 [40.0] | 4 [36.4] | 0 [0.0] |
| | PR^{$$} | 10 [15.6] | 10 [25.0] | 0 [0.0] | 0 [0.0] | 0 [0.0] |
| | CR^{$$} | 10 [15.6] | 10 [25.0] | 0 [0.0] | 0 [0.0] | 0 [0.0] |

*Mean [Min; Max];
^{$}NA: Not Available;
^{$$}PD: Progression Disease,
SD: Stable Disease,
PR: Partial Response,
CR: Complete Response

CD95/Fas and CD274/PD-L1 expression levels on CD4⁺ and CD8⁺ circulating T cells respectively were analyzed on frozen PBMCs at diagnosis before the first administration of 3 mg/kg of ipilimumab and their expressions were correlated with clinical outcome. Importantly, the CD95 membrane expression on CD4⁺ T cells analyzed in 64 patients was lower at diagnosis in patients developing partial and complete responses than in those exhibiting stable or progressive disease at 3 months of ipilimumab and confirmed with the ROC curve (FIG. 8D, up and down panels). Segregating the cohort of stage III-IV MM according to the median value of CD95 expression on CD4⁺ T cells allowed to conclude that both PFS and OS are significantly higher in patients presenting with low (<median) values at diagnosis prior to ipilimumab (FIG. 8E, up and down panels). The second parameter could only be analyzed in 24 patients and they also concluded that lower PD-L1 expression levels on CD8⁺ T cells (but not CD4⁺ T cells) at diagnosis were associated with stable disease while higher levels tented to predict progression (FIG. 10 I).

Since these two immunometrics appeared to have predictive value for the response to ipilimumab, inventors excluded the possibility that they could also convey a prognosis value in stage III-IV MM by analyzing a retrospective cohort of 39 MM prior to the era of immunomodulators. Indeed, Fas/CD95 expression on CD4⁺ T cells was not associated with time to progression (PFS) nor overall survival (OS) (FIG. 8F). However, while the percentages of PD-L1 membrane expression on CD8⁺ (or CD4⁺) T lymphocytes failed to predict survival, the mean fluorescence intensity of PD-L1 was of clinical significance for PFS and OS (FIGS. 10 J and K and Jacquelot et al. JID in press).

Next, inventors carried out the validation of the predictive value for beneficial clinical outcome of the CD137 expression on circulating CD8⁺ T cells at diagnosis for the toxic combination of ipilimumab and nivolumab, administered in a Phase II adjuvant trial comparing the efficacy of nivolumab alone versus combined with ipilimumab in stage III MM. The expression levels of CD137 on circulating CD8⁺ T cells in this American cohort of patients was within the range of patients described in the French cohort (FIG. 15). Interestingly, stage III MM patients benefiting from the combination mAb therapy expressed much higher levels of CD137 on their circulating CD8⁺ T cells at enrolment in the Phase II adjuvant trial, compared with the levels in patients doomed to relapse (FIG. 16A). Importantly, this biomarker did not predict relapse in nivolumab-treated stage III MM, as anticipated from our correlative matrices (FIG. 16B).Altogether, the ex vivo mLN assay as well as the preselected predictive biomarkers of response or resistance to the mAbs ex vivo allowed to securely identify patients proned to respond or resist to the proposed therapy and represent functional pharmacodynamics biomarkers.

### Discussion

For the first time, inventors present a functional method called “the ex vivo mLN assay” capable of assessing the reactivity of tumor infiltrating immune effectors (T and NK cells) during a stimulation with various immune checkpoint blocking or agonistic immunostimulating mAb and their combinations coupled to a paired blood and tumor immune profiling of mLN in stage III MM with the final aim to correlate immune fingerprints with clinical parameters (21, 22).

First, they concluded that the method was feasible for almost all mLN specimen containing at least 10 million cells (37/46 were successfully performed and contained enough cells for the “ex vivo mLN assay”) but could be downscaled at the level of a biopsy if only one or 2 mAb should be tested. The method was also reliable in that both negative controls (18 hrs or 5 days-incubation in the absence of stimulus or in Ig control antibodies) allowed the basal assessment of T cell functions without non specific backgrounds, and positive controls (rIL-2 or IFN type 1) almost invariably triggered effector (and Treg) proliferation and Cxcl10 release respectively in all patients. This method analysed supposingly the most important dynamic T and NK cell parameters relevant to effector functions against cancer, such as proliferation and intracellular production of Th1 cytokines as well as Treg proportions in the coculture model system. Hence, to be on the safe side, inventors arbitrarily set up two independent criteria per mAb or condition of stimulation to score the response as “positive”, when a >1.5 fold change compared with the two negative controls was achieved. Finally, considering that in patients, these immunomodulators may act, not just at the level of tumor deposits or tumor draining LN but also in other lymphoid compartments (such as bone marrow, non-draining LN, gut, etc.), they monitored cytokine and chemokine release as surrogate markers for effector cell trafficking or homing to inflammatory sites. This mLN ex vivo assay could also be run from frozen specimen (not shown).

The findings indicated that mLN reactivity to immunomodulators was personalized in that i) a precise and specific typification of immune activation for each mAb or their combinations was not possible, in contrast to generalizable responses to rIL-2 or rIFN type 1, ii) each individual exhibited a specific pattern of response to the panel of stimulatory agents, a clustering/stratification of patients being impossible to establish on this cohort. Interestingly, inventors' long term expertise in this “ex vivo tumor restimulation assay” underscores the importance of a peculiar tumor microenvironment in the functional outcome. Indeed, GIST responded best to anti-IL-10 or anti-TRAIL Ab or rIFNa2a than to anti-PD-1 or anti-CTLA-4 Ab (Rusakiewicz et al, OncoImmunology, in press).

The most prominent markers helping the decision making for gearing therapy to ICB are not the obvious candidates. Hence, CD95 expression (and not CTLA-4) on CD4⁺ T cells is crucial to predict resistance to anti-CTLA-4 Ab, while CD137 in circulating CD8⁺ T cells is important for the reactivity to the combination of anti PD-1 (aPD-1) and anti CTLA-4 (aTLA-4) Ab. The clinical significance of CD95/CD95L has been largely investigated in various human malignancies (23-30). Notably, in breast cancer and melanoma, serum soluble CD95 or CD95L is associated with disease dissemination and dismal prognosis. A mechanism has been proposed in triple negative breast cancers where sCD95L levels are higher than in other breast cancer subtypes and dictate metastatic dissemination whereby metalloproteases-mediated cleavage of CD95L expressed by endothelial cells engage an unconventional CD95 signaling pathway involving EGFR and the Src kinase c-yes, leading to migration of breast tumor cells and not apoptosis (31). Primary and metastatic melanoma lesions express high levels of CD95 and CD95L (28) and melanoma reactive T cells resist to CD95L mediated cell death (30). Here inventors show that membrane associated CD95 on CD4⁺ T cells is associated with an activated and/or exhausted phenotype of TEM and TCM in blood and lesions and that it does not convey a prognostic value. However, low CD4⁺CD95⁺ T cell counts appeared to predict responses to ex vivo stimulation with anti-CTLA-4 Ab, and to in vivo administrations of ipilimumab at 3 months. It would be of utmost interest to assess whether anti-CTLA-4 Ab somehow prevent the shedding of CD95 and/or its ligand from the lymphocyte membrane, preventing the deleterious effects of it soluble form.

High PD-L1 expression on circulating CD8⁺ T cells (but not CD4⁺ T cells and maybe due to the low number of patients tested (N=23)) also predicted poor clinical outcome in MM receiving ipilimumab. This result is in line with the recent discovery of a cell autonomous role of the PD-1 signaling pathway on melanoma cells. Indeed, overexpression of the PD-1 receptor or engagement of melanoma-PD-1 by its ligand, PD-L1, in melanoma cells could enhance tumorigenicity in mice. Conversely, PD-L1 inhibition in melanoma or knockout of host PD-L1 both reduced tumor aggressiveness of PD-1 expressing melanomas. The authors showed that the melanoma intrinsic PD-1 expression modulated downstream effectors of the mTOR signaling (15). Inventors postulate that type I IFN enriched tumor microenvironment might regulate PD-L1 expression on surrounding CD8⁺ CTLs that in turn could engage with neighbouring melanoma, preventing death and promoting dissemination during ipiliumab therapy. This mechanism could represent another reason explaining the additive effects of the anti-CTLA-4+anti-PD-1 Ab combination.

These two novel predictive immunometrics have to be included in the long list of putative biomarkers potentially relevant for this ICB. Inventors' previous experiences suggested that high LDH levels and sCD25 concentrations in the serum negatively predicted TTP in ipilimumab treated-stage IV MM (32) while CLA expressing CD8⁺ TEM represented a pharmacodynamic trait of sensitivity to CTLA4 blockade (Jacquelot, JCI in press). While HLA subtype (33), genetic polymorphisms (34), and absolute lymphocyte counts (35) have not been validated, a number of alternative parameters such as high baseline levels of Foxp3, IDO expression (34) and increase TILs and TH1 cells at baseline (36) or MDSC numbers (37) or T cell ICOS expression as pharmacodynamic markers (38) or more recently a high mutational load and neoantigen landscape (39) have all to be prospectively studied.

Biomarkers of response to anti-PD-1/PD-L1 Ab have been largely studied and may be considered as promising for future prospective validation. Selective CD8⁺ T cell infiltrations preceding PD-1 blockade, often correlated with PD-L1 expression and with a precise geodistribution at the tumor invasive margins appeared to predict OR in stage IV melanoma (40-42). The immunohistochemical determination of PD-L1 expression, although lacking a methodology for standardization and subjected to variegated expression depending on timing and biopsy sites, may also influence the response to PD-1 blockade and guide the choice between PD-1 versus CTLA-4+PD-1 coblockade (41, 43). Once again, a high mutational load is also associated with clinical responses to PD-1 blockade (39, 44). Inventors herein provide advantageous new blood biomarkers (in particular PD-1 expression on CD4⁺ T cells or the CD8/Treg ratio in blood).

### Material and Methods

Patients and Cohorts Characteristics. Prospective Cohort of 37 Patients.

Patients over 18 years old from Gustave Roussy Cancer Campus and Centre Hospitalier Lyon Sud, with histologically confirmed metastatic and/or resectable melanoma provided written informed consents according with protocols reviewed and approved by institutional ethic committee including the investigator-sponsored MSN study (NCT02105168). Retrospective cohort of 64 patients. University of Tubingen cohort Blood was collected and markers were assessed before Ipilimumab and IL-2 injections from 3 patients participating in a phase II study evaluating safety and efficacy of combined ipilimumab and intratumoral IL-2 treatment in pretreated patients with stage IV melanoma (clinical trial number: NCT01480323). University of Siena cohort. Blood samples were collected before ipilimumab treatment of unresectable stage III and stage IV melanoma at the University Hospital of Siena between July 2011 and June 2015. Markers were assessed after thawing samples. Memorial Sloan Kettering Cancer Center cohort. Blood samples were collected before injections of Ipilimumab from patients suffering of stage IV melanoma (clinical trial number: NCT00495066). Markers were assessed after thawing samples. University of Stanford cohort. Blood samples were collected before injections of Ipilimumab from patients participating in a study evaluating Ipilimumab in adjuvant. Markers were assessed on PBMC after thawing.

Peripheral Blood Mononuclear Cells (PBMC) Preparations.

Peripheral blood samples from 23 patients drawn just prior to surgery were carefully layered on top of a Ficoll-Hypaque density gradient media (PAA Laboratories). After washes, PBMC were counted and stained with appropriate Abs as described below and in the Table 4.

TABLE 4
List of monoclonal antibodies used for the Flow
Cytometry in the ex-vivo assay

| Name | Fluorochrome | Company | Reference | Clone |
|---|---|---|---|---|
| CD8 | FITC | BD | 555366 | RPA-T8 |
| CD4 | PerCP | BD | 345770 | SK3 |
| CD56 | PE Cy7 | Beckman | A21692 | N901 |
| CD3 | VioBlue | Miltenyi | 130-094-363 | OKT3 |
| Dead cells | Yellow | Invitrogen | L34957 | — |
| CD45 | APC AF750 | Beckman | A79392 | J.33 |
| TIM3 | APC | eBiosciences | 17-3109-42 | F38-2E2 |
| CD152 | PE | BD | 555853 | BNI3 |
| (CTLA-4) | | | | |
| CD137 | APC | Biolegend | 309810 | 4B4-1 |
| (CD137) | | | | |
| CD137L | PE | BD | 559446 | C65-485 |
| (CD137L) | | | | |
| CD274 | APC | Biolegend | 329708 | 29E.2A3 |
| (PD-L1) | | | | |
| CD95 (Fas) | APC | BD | 558814 | DX2 |
| CD178 (FasL) | PE | eBiosciences | 12-9919-42 | NOK-1 |
| CD69 | APC | BD | 555531 | FN50 |
| CD69 | PerCP | BioLegend | 310928 | FN50 |
| CD25 | PE | BD | 555432 | M-A251 |
| CD45RA | PE | BD | 555489 | HI100 |
| CD314 | PE | Miltenyi | 130-092-672 | BAT221 |
| (NKG2D) | | | | |
| CD279 (PD-1) | PE Cy7 | Beckman | A78885 | PD-1.3.5 |
| CD27 | APC | BD | 558664 | M-T271 |
| CD127 | APC | Miltenyi | 130-094-890 | MB15- |
| | | | | 18C9 |
| LAG3 | FITC | R&D | FAB2319F | Polyclonal |
| | | | | Ab |
| CD14 | FITC | BD | 555397 | M5E2 |
| CD15 | PB | BioLegend | 323022 | W6D3 |
| HLA DP/DR/ | APC | Miltenyi | 130-104-824 | REA332 |
| DQ | | | | |
| HLA DR | PB | Beckman | A74781 | Immu-357 |
| CD11c | PE Cy7 | BioLegend | 301608 | 3.9 |
| CD11b | PE Cy7 | BD | 557743 | ICRF44 |
| CD19 | PE Cy7 | BD | 557835 | SJ25C1 |
| CD20 | PE | Miltenyi | 130-091-109 | LT20 |
| TNFα | AF647 | Biolegend | 502916 | Mab11 |
| IFNγ | PE | BD | 559327 | B27 |
| Ki67 | PE | BD | 556027 | B56 |
| Foxp3 | APC | eBiosciences | 17-4776-42 | PCH101 |
| ICOS | PE | BD | 557802 | DX29 |
| CCR7 | BV421 | BioLegend | 353208 | G043H7 |
| CD40L | FITC | BD | 555699 | TRAP1 |

Tumor Infiltrated Lymphocyte (TIL) Preparations.

Resected mLN specimens from 37 MM were cut and placed in dissociation medium, which consisted of RPMI1640, 1% Penicillin/Streptomycine (PEST, GIBCO Invitrogen), Collagenase IV (501U/mL), Hyaluronidase (280 IU/mL), and DNAse I (30 IU/mL) (all from Sigma-Aldrich), and run on a gentle MACS Dissociator (Miltenyi Biotec). Dissociation time lasted one hour under mechanical rotation and did not influence the results of the phenotyping. Cell samples were diluted in PBS, passed through a cell strainer and centrifuged for 5 minutes at 1500 rpm. Cells were finally resuspended in PBS, counted, stained for flow cytometric analyses or resuspended in CryoMaxx medium (PAA Laboratories) for storage in liquid nitrogen. All mLN included in the study were histologically confirmed to be invaded.

Ex-Vivo MLN Assays.

Dissociated cells from mLN were incubated in two 48-well plates at 0.3×10⁶/ml in complete medium (RPMI 1640 supplemented with 10% human AB serum [Institut de Biotechnologie Jacques Boy], 1% Penicillin/Streptomycine (PEST, GIBCO Invitrogen), 1% L-glutamine (GIBCO Invitrogen) and 1% of sodium pyruvate (GIBCO Invitrogen)) and with isotype control, agonistics (CD137/CD137L) or antagonistic (PD-1/PD-L1, CTLA-4, Tim-3) mAbs or cytokines (IFNα2a [Roferon®, ROF], IL-2) or their combinations (PD-1+ROF, CTLA-4+ROF, PD-1+Tim-3, PD-1+CTLA-4) as described in the FIG. 1 and Tables 1 and 2. After 18 to 24 hrs of incubation with or without drugs, cells were stimulated with PMA (5 ng/ml) (Sigma), ionomycine (125 ng/ml) (Sigma) and BD Golgi Stop (4 μl per 6 ml) (BD Biosciences) for 3 to 5 hrs. Cells were then harvested, membrane stained to discriminate different lymphocyte subsets (Table 4), permeabilized with BD Cytofix/Cytoperm™ kit (BD Biosciences). Intracellular staining was performed with anti-IFN-γ-PE (BD Biosciences, clone B27) and anti-TNFα-AF647 (BioLegend, clone Mab11) mAbs. For the second plate, after 4 to 5 days of culture with or without drugs, cells were harvested and membrane stained, permeabilized with Foxp3/Transcription factor Fixation/Permeabilization kit (eBiosciences) and intranuclearly stained with anti-Ki67-PE (BD Biosciences, clone B56) and anti-Foxp3-APC (eBiosciences, clone PCH101) mAbs following the manufacturer's recommendations.

Flow Cytometric Analyses.

For membranous labeling, PBMC and TILs were stained with fluorochrome-coupled monoclonal antibodies (mAbs detailed in Table 4 and 5), incubated for 20 min at 4° C. and washed. Cell samples were acquired on a Cyan ADP 9-color flow cytometer (Beckman Coulter) with single-stained antibody-capturing beads used for compensation (Compbeads, BD Biosciences). Data were analyzed with Flowjo software v7.6.2 (Tree Star, Inc).

TABLE 5
List of monoclonal antibodies used for chemokine receptors analysis

| Specificity | Fluorochrome | Ab clone | Company | Reference |
|---|---|---|---|---|
| CXCR5 | AF488 | RF8B2 | BD | 558112 |
| CLA | FITC | HECA-452 | BD | 561987 |
| CRTH2 | FITC | BM16 | BD | 561659 |
| CD103 | PE | Ber-ACT8 | BD | 550260 |
| CCR10 | PE | 314305 | R&D | FAB3478P |
| CD4 | PE-CF594 | RPA-T4 | BD | 562281 |
| CD8 | PerCP | SK1 | BD | 345774 |
| CCR9 | PerCP Cy5.5 | BL/CCR9 | Biolegend | 346303 |
| CXCR4 | PerCP Cy5.5 | 12G5 | Biolegend | 306516 |
| CXCR3 | PE Cy7 | 1C6/CXCR3 | BD | 560831 |
| CD4 | PE Cy7 | SK3 | BD | 557852 |
| CCR7 | BV421 | G043H7 | Biolegend | 353208 |
| CD14 | V500 | M5E2 | BD | 561391 |
| CD15 | V500 | HI98 | BD | 561585 |
| CD16 | V500 | 3G8 | BD | 561394 |
| CD19 | V500 | HIB19 | BD | 561121 |
| CD8b | APC | 2ST8.5H7 | BD | 641058 |
| CCR6 | AF647 | 11A9 | BD | 560466 |
| CD45RA | APC-H7 | HI100 | BD | 560674 |
| Dead Cells | Yellow | — | Invitrogen | L34959 |

Cytokines and Chemokines Measurements.

Supernatants from cultured cells were monitored using the human Th1/Th2/Th9/Th17/Th22 13-plex RTU FlowCytomix Kit (eBiosciences), and human Chemokine 6 plex kit FlowCytomix (eBiosciences) according to the manufacturer's instructions and acquired on a Cyan ADP 9-color flow cytometer (Beckman Coulter). Analyses were performed by Flowcytomix Pro 3.0 Software (eBiosciences). Moreover some measurements were done by ELISA with IFNg (BioLegend), IL-9 (BioLegend), TNFα (BD Biosciences), CCL2 (BD Biosciences), CCL3 (R&D Systems), CCL4 (R&D Systems), CCL5 (R&D Systems) and CXCL10 (BD Biosciences) kits in accordance with manufacturer's recommendations.

Statistics.

Data analyses and representations were performed within the statistical environment R (see Worldwide Website: R-project.org/). In all, 124 (blood) and 128 (tumor) parameters were considered for analyses and reporting. All remaining calculations were performed within R. Individual data points representing the measurement from one patient are systematically graphed alongside with the box and whiskers plot calculated from the corresponding distribution. Comparisons between clinical groups were performed by beta regression for parameters expressed in percentage and by linear modeling for the other parameters and ratios after log transformation. Dispersion was allowed to differ between groups and contrasts of interest are back-transformed and presented as ratios. Overall Survival (OS) and Progression Free Survival (PFS) determined from the date of sampling were used as the primary end-points. Survival curves were estimated by the Kaplan-Meier product-limit method. Survival distributions were compared by Firth's penalized-likelihood Cox regression after adjusting for the BRAF status, gender, number of metastatic lymph nodes, lactate deshydrogenase levels (LDH) thresholded at 250 ui/L and disease stage. Unless stated, p-values are two-sided and 95% confidence intervals for the statistic of interest are reported. Effectiveness of the biomarker was determined from the 0.632 corrected bootstrap (B=199) after log-concave density smoothing of the AUC under the ROC curve. The empirical ROC curve and corresponding AUC are reported and graphed together with the smoothed curves. Optimal cut-off corresponds to the maximum effectiveness of the biomarker followed the Youden index and the confidence intervals were determined on the same bootstrap samples as for the AUC.

### Example 2—CLA Expression on CD8⁺ T Cells Assessed 3 Weeks after the First Injection Predicts the Response to Ipilimumab Treatment

### Significant Changes During Immune Checkpoint Blockade by Ipilimumab

CTLA4 blockade by the FDA- and EMEA-approved drug ipilimumab induces significant and prolonged (>7 years) antitumor effects in about 20% of metastatic melanoma (MMel) (19, 20). Inventors analyzed all the CC and CXC chemokine receptors described herein (Table 5) in 47 patients diagnosed with stage IV MM treated with ipilimumab (mainly 3 mg/kg (87%)), enrolled at four clinical centers (detailed in Jacquelot et al, JCI in press). Interestingly, although most of the above detailed markers were analyzed, only CLA expression on CD8⁺ TEM [cell numbers (FIG. 14A) as well as proportions (FIG. 14B)] and not on the CD4⁺ T cell population (FIGS. 14C and D) monitored after one ipilimumab administration was significantly associated with clinical responses evaluated by RECIST criteria at 3 months post-treatment start, taking into account the inter-centers variations (FIG. 14A left and right panel and AUC=0.835; 14B, left and right panel and AUC=0.752). Altogether, CTLA-4 blockade modulated the numbers and/or proportions of CLA⁺TEM, and such changes constitute pharmacodynamic markers or predictors of therapeutic response.

### Example 3—Predictors of Responses to Immune Checkpoint Blockade in Advanced Melanoma

Immune checkpoint blockers (ICB) have become pivotal therapies in the clinical armory against metastatic melanoma (MMel). Given the frequency of immune related-adverse events and increasing use of ICB, predictors of response to CTLA-4 and/or PD-1 blockade represent unmet clinical needs. Using a systems biology-based approach on assessment of 779 paired blood and tumor markers in 37 stage III MMel patients, inventors analyzed correlates between blood immune parameters and the functional immune reactivity of tumor-infiltrating cells after ex vivo exposure to ICB. Based on this assay, they retrospectively validated in 8 cohorts, enrolling 190 MMel patients, that high PD-L1 expression on peripheral T cells was the best marker predicting shorter progression free- and overall-survival to ipilimumab. Moreover, detectable CD137 on circulating CD8⁺ T cells was associated with the disease-free status of resected stage III MMel patients after adjuvant ipilimumab+nivolumab (but not nivolumab alone).

The recent development of immune checkpoint blockers (ICB) has rekindled interest in the field of immune cancer therapies (3,4). Cancer vaccines (5), adoptive T cell transfer and CAR T cells (6,7), bispecific antibodies (8), ICBs (9,10) and oncolytic viruses (11) have come of age and many immune agents have recently entered the oncological armory. However, to date, immunotherapy has only been shown to provide durable clinical benefit in a fraction of patients. The recent characterization of multiple immune resistance mechanisms by which tumors can evade the immune system has fueled the development of novel agents that circumvent such limitations, targeting new “immune checkpoints”. It is likely that the use of combination strategies will increase the number of cancer patients that might benefit from immunotherapy (12). Nonetheless, several critical problems remain to be solved. First, the scientific rationale supporting the use of combinatorial regimens needs to be defined. Second, it must be determined whether the future of immuno-oncology (I-O) will rely on patient stratification in large cohorts or will be personalized to each patient. Depending on tumor characteristics (e.g., PD-L1 or PD-1 expression on tumor cells for anti-PD-1 mAb (13-15), HMGB1 and LC3B for immunogenic chemotherapy (16), or tumor microenvironment hallmarks such as IDO expression (17), macrophage density (18), tumor-infiltrating lymphocytes [TIL], or Th1 fingerprints (19)), one might envisage more specific and individualized I-O clinical management strategies. Third, predictive immune profiles or biomarkers will need to be validated prospectively to guide I-O utilization in a personalized or stratified manner.

Inventors attempted to address some of these questions in patients with stage III melanoma (45), given that (i) optimizing adjuvant I-O therapies for metastatic melanoma (MMel) remains an unmet clinical need, (ii) MMel represents a clinical niche for the development of many mAbs and ICBs, (iii) in these patients, metastatic lymph nodes (mLN) are surgically resected, enabling immunological investigations, and (iv) immune prognostic parameters have been recently described in stage III/IV MMel (46, 47). The tumor microenvironment has a complex regulation. Each checkpoint/co-stimulatory pathway displays an independent mechanism of action and this call for a comprehensive analysis of their mode of action in the tumor microenvironment in a given patient to design appropriate combinatorial approaches and to discover specific biomarkers of response. Herein, inventors used a systems biology-based approach aimed at defining relevant immunometrics for prediction of an in situ response to cytokines and monoclonal antibodies (mAb) (i.e., agonists and blockers of immune checkpoints) in patients with resected stage III melanoma. They describe a suitable “ex vivo metastatic lymph node (mLN) assay” (see example 1), and through this assay they demonstrate, in multivariate analyses performed on 8 pooled cohorts gathering 190 samples of unresectable stage III and IV melanoma. They demonstrated that the best markers predicting resistance to ipilimumab were high PD-L1 expression in peripheral blood CD4⁺ and CD8⁺ T cells (for progression free survival (PFS) and overall survival (OS)), while in stage III melanoma, detectable CD137⁺CD8⁺ peripheral blood T cells predicted a lack of relapse with ipilimumab+nivolumab combination therapy. They conclude that i) the “ex vivo metastatic lymph node (mLN) assay” represents a suitable method to identify biomarkers for ICB, ii) PD-L1 expression on blood CD8⁺ T cells is a strong marker of resistance to CTLA4 blockade.

### Results

### Functional Immunological Assays on Ex Vivo Dissociated mLN.

The study population consisted of stage III MMel patients undergoing surgery for lymph node metastases, as previously described (46). Of these patients, one third presented with more than 3 involved LN at surgery, 55% had a mutated BRAF oncogene, >30% had thyroid dysfunction, and >50% were scheduled to undergo adjuvant therapy. Of primary lesions, 52% were ulcerated. After mechanical and enzymatic digestion of mLN (46), CD45⁻ cells represented 4-98±4.8% of all cells. The composition of tumor-infiltrating immune cells was analyzed by flow cytometry with gating on live cells in 39 tumor specimens that were paired with autologous peripheral blood cells. Analyses were based on a comprehensive immunophenotyping of 252 parameters per patient, featuring cell type, activation status, naïve or memory phenotype, and activating or inhibitory receptors or ligands. Inventors previously found that peripheral blood cell markers were as relevant as TIL immunotypes for prognosis, and parameters associated with lymphocyte exhaustion/suppression were associated with greater clinical significance compared to those related to activation or lineage (46). The next step consisted of analyzing the dynamics of these parameters after incubation with mAbs+/−cytokines on 37 patients. Comprehensive assessment of the reactivity of various subsets of infiltrating tumor cells targeting four functional axes, cytokines and their combinations is described (FIG. 1 and Table 2). A biological response to a given axis was scored “positive” when two independent readouts, reaching a >1.5 fold increase or decrease over two background levels (that of the medium and the isotype control Ab) were achieved. The inter-individual variability for specimen manipulation and flow cytometry analyses was minimal, as demonstrated by 2 specimens handled by the two first authors independently (FIG. 2). Charts depicting the overall relative levels of reactivity in “ex vivo responders” versus “ex vivo non-responders” for each biological readout and culture condition are presented in FIG. 3-5. As a positive control, IL-2 stimulation of mLN frequently induced T and NK cell proliferation, as well as cytokine release mostly by NK cells (FIG. 6, FIG. 3). Additionally, ex vivo stimulation with rIFNα2a led to high CXCL10 release (FIG. 6 and FIG. 3). mLN responding to PD-1 blockade more often exhibited T cell proliferation and chemokine release (CCL4, CCL5, CXCL9, and CXCL10) (FIG. 6 and FIG. 4). mLN responding to CTLA-4 blockade often demonstrated polyfunctional T cell activation and chemokine release (CCL4, CCL5 and CXCL9) (FIG. 6 and FIG. 4). mLN responding ex vivo to CTLA-4/PD-1 co-blockade typically showed NK cell proliferation and CXCL10 release (FIG. 6, FIG. 5). Anti-Tim-3 mAb often led to NK and CD4⁺ T cell proliferation, inflammatory cytokines and CCL4/CCL5 production (FIG. 6, FIG. 5). mLN responding to CD137/CD137L stimulation often exhibited CD8⁺ T cell proliferation and IFNγ production accompanied by IL-1β, IL-6, and TNFα release (FIG. 6, FIG. 5).

The Venn diagrams detailing the patterns of immune reactivities are depicted in FIG. 7. The proportions of mLN “ex vivo responding” to at least one I-O axis were approximately 30-50% and 50-60% for mAb combinations (Table 2, FIGS. 7a, b). The proportion of mLN “ex vivo responding” to both anti-CTLA-4 and anti-PD-1 mAb separately was 11/37 (30%), among which 45% failed to respond to concomitant blockade (Table 2). Sixty percent ( 17/28) of mLN “ex vivo responded” to agonistic anti-CD137/anti-CD137L mAb, among which 35% ( 6/17) failed to respond to any of the classical ICBs (anti-CTLA-4 or anti-PD-1 mAbs) (Table 2). The likelihood of “ex vivo response” to any alternate ICB or mAb combination in cases failing to respond to any one monotherapy or combination therapy is depicted in FIG. 7c. Altogether, our ex vivo mLN assay is a feasible test potentially allowing a diagnosis of prediction of ex vivo response to 11 conditions of stimulation.

### Predictive Biomarkers of Resistance to CTLA-4 Blockade

Inventors next addressed whether predictive biomarkers of a functional response obtained in the ex vivo mLN assay could be inferred from the 779 blood/tumor parameters. Very few statistically significant immune parameters predicting responses to CTLA-4 blockade could be found (FIG. 8G).

The stronger predictive biomarkers of ex vivo resistance to CTLA-4 blockade were elevated PD-L1 expression on circulating CD4⁺ T cells (FIG. 10C, AUC=0.79, p<0.01) and CD8⁺ T cells (FIG. 10A, AUC=0.76, p<0.03) but not in the tumor infiltrating lymphocytes (FIG. 19a-b). Other potential biomarkers such as CD95 expression (best significance in blood for CD8⁺ T cells, p=0.08, and in tumors for CD4⁺ T cells, p<0.01, FIG. 8C, 8B, 9A, 9C) were also selected in the model. Of note, CD95 membrane expression on CD4⁺ T cells was dominant in Treg and chronically activated CD4⁺ T cells as well as terminally differentiated effector CD8⁺ T cells (but not naïve T cells, FIGS. 11A1, A2), and highly correlated with HLA-DR and PD1 expressions (FIGS. 11B, C). Additionally, although retained in the statistical analyses, some biomarkers were not considered further due to the weak detectability (<2% expression) and low robustness of the flow cytometric analyses. Based on the ex vivo mLN functional assay and blood immunometrics, inventors hypothesized that PD-L1 and/or CD95 on circulating CD4⁺ and CD8⁺ T cells might predict resistance to ex vivo CTLA-4 blockade in MMel.

Ipilimumab not only improves overall survival in stage IV MMel but also impacts overall-survival, recurrence-free survival and distant metastasis-free survival in resected high-risk stage III melanoma (48, 49). In order to validate their immune biomarkers selected in the “ex vivo mLN assay”, inventors retrospectively analyzed this blood T cell phenotype, focusing on PD-L1 and CD95, in 8 cohorts from different centers including 190 unresectable stage III and IV MMel patients treated with 3 mg/kg (in 90% cases) of ipilimumab with a median follow-up of 30 months [95% CI: 26-34] (patients' characteristics presented in Table 7).

TABLE 7
Cohorts and patient's characteristics

| | | Overall | | | | | | | | |
|---|---|---|---|---|---|---|---|---|---|---|
| | | (190) | CA (19) | CH (16) | DE (3) | DK (67) | FR (15) | IT (10) | OR (20) | JE (40) |
| Gender | Female | 93 (49%) | 8 (42%) | 5 (31%) | 0 (0%) | 36 (54%) | 12 (80%) | 4 (40%) | 9 (45%) | 19 (47%) |
| | Male | 97 (51%) | 11 (58%) | 11 (69%) | 3 (100%) | 31 (46%) | 3 (20%) | 6 (60%) | 11 (55%) | 21 (53%) |
| Age | Mean | 61 (13) | 64 (12) | 58 (15) | 64 (11) | 63 (12) | 66 (14) | 56 (16) | 59 (13) | 58 (14) |
| | (SD) | | | | | | | | | |
| LDH | Low | 112 (62%) | 6 (32%) | 13 (100%) | 1 (33%) | 56 (85%) | 11 (73%) | 2 (22%) | 6 (30%) | 17 (47%) |
| | High | 69 (38%) | 13 (68%) | 0 (0%) | 2 (67%) | 10 (15%) | 4 (27%) | 7 (78%) | 14 (70%) | 19 (53%) |
| | Missing | 9 | 0 | 3 | 0 | 1 | 0 | 1 | 0 | 4 |
| Tumor stage | III | 13 (7%) | 1 (5%) | 2 (12%) | 0 (0%) | 0 (0%) | 8 (53%) | 1 (10%) | 1 (5%) | 0 (0%) |
| | IV | 177 (93%) | 18 (95%) | 14 (88%) | 3 (100%) | 67 (100%) | 7 (47%) | 9 (90%) | 19 (95%) | 40 (100%) |
| Tumor | PD | 127 (67%) | 11 (58%) | 13 (81%) | 3 (100%) | 42 (63%) | 2 (13%) | 6 (60%) | 17 (85%) | 33 (83%) |
| response | SD | 31 (16%) | 1 (5%) | 1 (6%) | 0 (0%) | 14 (21%) | 7 (47%) | 4 (40%) | 0 (0%) | 4 (10%) |
| | PR | 18 (9%) | 6 (32%) | 0 (0%) | 0 (0%) | 9 (13%) | 2 (13%) | 0 (0%) | 1 (5%) | 0 (0%) |
| | CR | 14 (7%) | 1 (5%) | 2 (12%) | 0 (0%) | 2 (3%) | 4 (27%) | 0 (0%) | 2 (10%) | 3 (8%) |
| Previous CT | Yes | 71 (42%) | 9 (47%) | 8 (50%) | 2 (67%) | 11 (16%) | 3 (20%) | 8 (80%) | 0 (0%) | 30 (75%) |
| | Missing | 20 | 0 | 0 | 0 | 0 | 0 | 0 | 20 | 0 |
| Previous IT | Yes | 66 (35%) | 4 (21%) | 1 (6%) | 1 (33%) | 19 (28%) | 11 (73%) | 3 (30%) | 14 (70%) | 13 (32%) |
| Previous PKI | Yes | 16 (9%) | 2 (11%) | 3 (19%) | 1 (33%) | 3 (4%) | 1 (7%) | 3 (30%) | 0 (0%) | 3 (8%) |
| | Missing | 20 | 0 | 0 | 0 | 0 | 0 | 0 | 20 | 0 |
| Ipilimumab | 3 mg/kg | 143 (88%) | 0 (0%) | 16 (100%) | 3 (100%) | 67 (100%) | 15 (100%) | 10 (100%) | 20 (100%) | 12 (100%) |
| dose | 10 mg/kg | 19 (12%) | 19 (100%) | 0 (0%) | 0 (0%) | 0 (0%) | 0 (0%) | 0 (0%) | 0 (0%) | 0 (0%) |
| | Missing | 28 | 0 | 0 | 0 | 0 | 0 | 0 | 0 | 28 |
| Co-treatment | GMCSF | 19 (10%) | 19 (100%) | 0 (0%) | 0 (0%) | 0 (0%) | 0 (0%) | 0 (0%) | 0 (0%) | 0 (0%) |
| | IL2 | 3 (2%) | 0 (0%) | 0 (0%) | 3 (100%) | 0 (0%) | 0 (0%) | 0 (0%) | 0 (0%) | 0 (0%) |

PD-L1 and CD95 were evaluated retrospectively at diagnosis in whole blood or PBMCs (after density gradient separation of cells) by flow cytometry gating on CD4⁺ and/or CD8⁺ T cells using a standardized methodology validated for all centers (either performed by inventors' laboratory, after thawing of cryopreserved cells or by the investigators themselves using inventors' antibodies and procedures). CD95 expression levels were higher in MMel compared with HV in blood T cells (FIG. 17a). Although variable according to sites and individuals, PD-L1 expression levels were highly detectable in circulating CD4⁺ (FIG. 17b) and CD8⁺ (FIG. 17c) T cells in stage III/IV MMel patients, while remaining below the threshold of confidence in healthy volunteers (“HV”, FIG. 17b-c). PD-L1 expression on CD8⁺ T cells was independent of LDH (p=0.71, comparing high versus low LDH levels, not shown) and of metastases localization (FIG. 17d), highly correlated with that on CD4⁺ T cells (rho=0.83, FIG. 17e) but not with CD95 expression (FIG. 17f, rho between 0.01 and 0.12).

Next, PD-L1 and CD95 biomarkers have been analyzed on a continuous scale.

First, the tumor response evaluated at 3 months was categorized into 4 groups: progressive disease (PD, n=127 (67%), stable disease (SD, n=31, 16%), partial response (PR, n=18, 9%) and complete response (CR, n=14, 7%) (Table 7). The chosen binary outcome for the logistic regression model was: PD (n=127) versus SD+PR+CR (n=63). Table 8 shows the impact of clinical covariates on tumor response and survival endpoints (PFS and OS).

TABLE 8
Clinical prognostic parameters for Ipilimumab responses
(PD vs SD + PR + CR), OS and PFS

| | Tumor | Overall | Progression - |
|---|---|---|---|
| | response | survival | free survival |
| Age | 1.002 | 0.992 | 0.992 |
| | [0.997; 1.007] | [0.977; 1.006] | [0.979; 1.006] |
| | P = 0.468 | P = 0.268 | P = 0.253 |
| Gender | 0.94 | 1.09 | 1.26 |
| (ref = ‘female’) | [0.83; 1.07] | [0.76; 1.58] | [0.89; 1.77] |
| | P = 0.38 | P = 0.63 | P = 0.20 |
| LDH | 0.87 | 2.31 | 1.51 |
| (ref = ‘low’) | [0.74; 1.01] | [1.47; 3.62] | [0.96; 2.38] |
| | P = 0.068 | P < 0.001 | P = 0.073 |
| Previous CT | 0.92 | 1.62 | 1.01 |
| (ref = ‘no’) | [0.79; 1.08] | [1.02; 2.58] | [0.68; 1.48] |
| | P = 0.32 | P = 0.043 | P = 0.97 |
| Previous IT | 1.08 | 0.71 | 0.77 |
| (ref = ‘no’) | [0.94; 1.25] | [0.47; 1.08] | [0.53; 1.13] |
| | P = 0.27 | P = 0.11 | P = 0.18 |
| Previous PKI | 0.74 | 2.04 | 1.95 |
| (ref = ‘no’) | [0.58; 0.93] | [1.01; 4.09] | [1.04; 3.67] |
| | P = 0.012 | P = 0.046 | P = 0.038 |
| Tumor stage | 1.03 | 1.01 | 0.55 |
| (ref = ‘stage III’) | [0.77; 1.40] | [0.29; 3.47] | [0.21; 1.43] |
| | P = 0.83 | P = 0.99 | P = 0.22 |

All models were stratified on the center. Odds Ratio (for the tumor response) and Hazard Ratio (for the survival endpoints) with 95% confidence intervals. CT: chemotherapy and/or radiation, IT: immunotherapy, PKI: protein kinase inhibitors.

Even if the gender, the age and the tumor stage were not significant in the univariate analysis, inventors kept these variables in the final model as they are recognized as potential prognostic factors. Hence, for the following analyses, the final models were stratified based on the centers and adjusted for LDH (“low or high”, meaning below or above the normal value for each individual clinical center), previous chemotherapy (“yes” or “no”), previous immunotherapy (“yes” or “no”), previous protein kinase inhibitor (“yes” or “no”), gender (“male” or “female”), age (continuous scale) and tumor stage (III or IV). When considering the predictive value of CD95 or PD-L1 in blood CD4⁺ and CD8⁺ T cells for response rates assessed after 4 cycles of ipilimumab, and adjusting for all of the other clinical variables (Table 8), inventors found that the only immunometrics associated with clinical responses at 12 weeks were CD95 on CD4⁺ T cells (FIG. 18a, p=0.023 in univariate analysis, p=0.12 after adjustment) and PD-L1 on CD8⁺ T cells (FIG. 18b, p=0.068 in multivariate analyses).

Next, inventors analyzed the impact of those biomarkers on PFS (in 169 MMel including 143 events) and OS (in 189 MMel including 121 events) (FIG. 20). The most relevant clinical parameters impacting PFS was history of protein kinase inhibition (PKI), while LDH and previous chemotherapy/radiotherapy or PKI influenced OS (Table 8). High PD-L1 expression (considered as a continuous variable) in circulating CD4⁺ T cells and to a lesser extent in CD8⁺ T cells represented the strongest parameter retained in our model for shorter PFS after CTLA-4 blockade (Table 6, p=0.009 for PD-L1⁺CD4⁺ and p=0.056 for PD-L1⁺CD8⁺ in multivariate analyses).

TABLE 6
Association between CD95 and PD-L1 (continuous scale) and the
progression free- and overall survivals

| | | | | |
|---|---|---|---|---|
| Model | CD95.CD4 | CD95.CD8 | PDL1.CD4 | PDL1.CD8 |

Progression free survival

| | | | | |
|---|---|---|---|---|
| Univariate | 1.007 | 0.996 | 1.021 | 1.021 |
| | [0.997; 1.017] | [0.986; 1.006] | [0.999; 1.043] | [1.001; 1.042] |
| | P = 0.19 | P = 0.44 | P = 0.057 | P = 0.040 |
| Stratify on center | 1.001 | 0.998 | 1.022 | 1.016 |
| | [0.989; 1.012] | [0.986; 1.009] | [0.993; 1.051] | [0.986; 1.046] |
| | P = 0.92 | P = 0.69 | P = 0.15 | P = 0.30 |
| Stratify on center | 1.004 | 0.999 | 1.044 | 1.032 |
| Adjust for LDH, | [0.991; 1.017] | [0.984; 1.015] | [1.011; 1.079] | [0.999; 1.065] |
| gender age, tumor | P = 0.59 | P = 0.93 | P = 0.009 | P = 0.056 |
| stage, CT, IT and | | | | |
| PKI* | | | | |

Overall Survival

| | | | | |
|---|---|---|---|---|
| Univariate | 1.010 | 1.013 | 1.036 | 1.043 |
| | [0.999; 1.022] | [1.001; 1.025] | [1.012; 1.059] | [1.023; 1.063] |
| | P = 0.082 | P = 0.031 | P = 0.003 | P < 0.001 |
| Stratify on center | 1.009 | 1.001 | 1.027 | 1.045 |
| | [0.996; 1.022] | [0.989; 1.014] | [0.993; 1.062] | [1.015; 1.076] |
| | P = 0.16 | P = 0.83 | P = 0.12 | P = 0.003 |
| Stratify on center | 1.010 | 1.009 | 1.041 | 1.053 |
| Adjust for LDH, | [0.994; 1.027] | [0.991; 1.028] | [0.995; 1.089] | [1.012; 1.096] |
| gender, age, tumor | P = 0.21 | P = 0.33 | P = 0.081 | P = 0.011 |
| stage, CT, IT and | | | | |
| PKI* | | | | |

Hazard ratios and 95% confidence intervals. Final model designated with an *.

Similarly, elevated levels of those biomarkers on pre-treatment T cells were also significantly associated with OS with PD-L1⁺CD8⁺ T cells (FIG. 10G, E, p=0.011 in multivariate analyses) outperforming PD-L1⁺CD4⁺ T cells (p=0.081 in multivariate analyses) (Table 6). Of note, low CD95 expression on circulating CD8⁺ T cells was protective (although low numbers of patients presented with an expression level <40%) (FIG. 19c) while the selection of a cut-off value >70% (on the basis of the upper limit of positivity found in HV, FIG. 17a) suggested that high levels of CD95 on CD4⁺ T cells was not only associated with therapeutic resistance (FIG. 18a) but also with shorter OS (FIG. 19d).

Moreover, a combination of these two markers, PD-L1⁺/CD8⁺ (according to the median value) and CD95⁺/CD4⁺ (according to the cut-off value at 70%) was feasible according to the low coefficient of correlation between these parameters enabling a segregation of the cohort into 4 arms with patients harboring both a high expression of PD-L1 and CD95 who were found to be associated with a shorter overall survival (FIG. 21)

Altogether, inventors' data indicate that high frequencies of circulating PD-L1⁺ T cells predict resistance to CTLA-4 blockade (mostly 3 mg/kg) for RR, PFS and OS in unresectable stage III and IV MMel.

### Predictive Biomarkers of Response to CTLA-4+PD-1 Co-Blockade

The regimen of ipilimumab and nivolumab has demonstrated impressive clinical benefit in MMel (objective response rate [ORR]>60% with a PFS>11 months), but is also associated with a high rate of immune related adverse events (>50% grade 3-4 events) (20). This supports further investigation into biomarkers which may predict which patient may derive the most benefit to spare primarily resistant patients the toxicity of the treatment. Given that the proportion of mLN that respond to both anti-CTLA-4 and anti-PD-1 mAb separately was 11/37 (29%), among which 45% failed to respond to combined blockade (Table 2, FIG. 7), inventors hypothesized that predictive biomarkers of response to this combination would have different immunometrics than those identified for anti-CTLA-4 blockade. Again, few immune parameters in blood and tumors were found to be associated with functional responses to the co-blockade (FIGS. 13A2, 15A, 15B, 13F). The two superior immunometrics retained in the assay of 779 variables were the expression levels of CD137/4-1BB on circulating CD4⁺ and CD8⁺ T lymphocytes (FIG. 15A, B). Detectable expression levels of CD137 in blood and tumor CD8⁺ T lymphocytes (and to a lesser extent in CD4⁺ T cells) at diagnosis were correlated to response to combined anti-PD-1/CTLA-4 mAbs (FIG. 15A-D). Based on these findings obtained in blood and tumor, inventors hypothesized that CD137 critically impacted sensitivity to CTLA-4/PD-1 co-blockade in MMel.

To validate the predictive value of CD137 expression on circulating CD8⁺ T cells at baseline for clinical benefit from the combination of ipilimumab and nivolumab, inventors analyzed this parameter in PBMCs obtained from a phase II adjuvant trial assessing the efficacy of nivolumab and ipilimumab combination therapy in resected stage IIIc and IV MMel. The median follow up of this study was 13 months. The expression levels of CD137 on circulating CD8⁺ T cells at baseline in this cohort of patients was within the range of those described above in patients with metastatic disease (FIG. 16A, B). Interestingly, stage III MMel patients with resected high risk disease who did not relapse after combination therapy expressed much higher levels of CD137 on their circulating CD8⁺ T cells at enrolment in the Phase II adjuvant trial, compared with the levels in patients who had a relapse (p=0.004) (FIG. 16A).

Of note, CD137 expression on CD8⁺ T cells did not predict relapse in patients with high-risk resected melanoma treated with nivolumab alone as anticipated from our correlative matrices (FIG. 16B). To analyze which biomarker was best associated with clinical outcome to PD-1 blockade, inventors returned to the ex vivo mLN assay described above. The best immunometrics obtained on circulating T cells and retained in the model of 779 variables were (i) PD-1 expression levels on CD4⁺ T cells, (ii) the ratio between CD8⁺ lymphocytes and CD127^{low}CD25^{high} CD4⁺ Treg cells, (iii) PD-L1 expression on CD4⁺ and CD8⁺ T cells as shown for ipilimumab (FIG. 12A2). Indeed, higher expression levels of PD-1 (>20%) in circulating CD4⁺ T cells at diagnosis was associated with the likelihood to respond in the ex vivo mLN functional assays using anti-PD-1 mAb (but not other mAbs; p<0.02, AUC=0.75) (FIG. 12B). A CD8⁺ T cell/Treg ratio >5 also tended to predict ex vivo reactivity of mLN to PD-1 blockade (but not to another I-O axis; p<0.06, AUC=0.73) (FIG. 12D). Similar to CTLA-4 blockade, lower expression levels of PD-L1 on circulating CD4⁺ and CD8⁺ T cells were associated with ex-vivo reactivity of PD-1 blockade (FIG. 12H). Neither PD-1 expression on CD4⁺ T cells nor the CD8⁺ T cell/Treg ratio were prognostic factors associated with PFS or OS before the era of ICBs (not shown).

Altogether, this study demonstrates that the ex vivo mLN assay as well as the preselected predictive biomarkers of response or resistance to the mAbs may the identification of patients likely to respond to fail the proposed therapy.

### Discussion

Inventors describe new predictive biomarkers of response to CTLA-4 blockade and to effective but potentially toxic combination therapy composed of anti-CTLA-4+anti-PD-1 mAbs. These results are based on a functional method herein called “the ex vivo mLN assay”, capable of assessing the reactivity of tumor infiltrating immune effectors (T and NK cells) during stimulation with various ICB or agonistic mAbs and their combinations. This was coupled with a paired blood and tumor immune profiling of mLN in stage III MMel with the intention of correlating immune fingerprints with clinical parameters(21, 22). Inventors elucidated the relevance of PD-L1 expression on circulating T cells for the prediction of resistance to ipilimumab, alone or in combination with IL-2 or GM-CSF. Moreover, their study shows that detectable levels of CD137 on circulating CD8⁺ T cells after LN or metastatic resection in stage IIIc and IV melanoma tends to predict longer PFS for the anti-CTLA-4+anti-PD-1 co-blockade.

The ex vivo mLN assay was feasible for almost all mLN specimens containing at least 10⁷ cells (37/46 were successfully performed and contained enough cells for the “ex vivo mLN assay”). Of note, this method could be downscaled to the size of a biopsy if only 1 or 2 mAbs had to be tested. The method is also reliable in that the two negative controls used (18-24 h or a 4-5 day incubation in the absence of stimulus or in the presence of Ig control mAb) allow the basal assessment of T cell functions to be determined (46) with low non-specific backgrounds. The high dose rIL-2 and rIFNα2a positive controls almost invariably triggered effector (and Treg) proliferation and CXCL10 release, respectively, in all patients. Inventors showed that this method can analyze important dynamic T and NK cell parameters relevant to effector functions against cancer, such as proliferation and release of Th1 cytokines as well as proportions of Tregs in the co-culture system. Cytokine and chemokine release could be considered as surrogate markers for effector cell trafficking or homing to inflammatory sites.

The findings from inventors' study indicate that the mLN reactivity to immunomodulators is specific for each patient since (i) a precise and specific pattern of immune activation for each mAb or their various combinations across patients was not possible, in contrast to generalizable responses to rIL-2 or rIFNα2a; (ii) each individual patient exhibited a specific pattern of response to the panel of stimulatory agents, therefore a clustering/stratification of patients was impossible to establish in this cohort. Interestingly, their long-term expertise with this ex vivo tumor restimulation assay underscores the relevance of the tumor microenvironment in dictating the functional outcome. Indeed, GIST responded best to anti-IL-10 or anti-TRAIL mAbs or rIFNα2a, rather than to anti-PD-1 or anti-CTLA-4 mAbs (50).

Inventors' study also uncovers, for the first time, two biomarkers of resistance or response to I-O regimens: ipilimumab alone or combined with PD-1 blockade. Herein, inventors found that the most prominent markers predicting response to such regimens are not the obvious candidates. PD-L1 (and not CTLA-4) on T cells was found crucial for the prediction of resistance to anti-CTLA-4 mAb, whereas CD137 expression on circulating CD8⁺ T cells appears a promising predictor of long term (>13 months) relapse-free survival mediated by the combination of anti-PD-1 and anti-CTLA-4 mAbs in the adjuvant setting.

Most previous biomarker studies with PD-1/PD-L1 antibodies have focused on the prognostic significance of PD-L1 (and/or PD-L2) expression on tumor cells or myeloid cells of the TME. Expression of both PD-L1 and PD-L2 significantly correlated with increasing densities of immune cells in the tumor specimens and with immunotype. Positive PD-L2 expression alone or combination with PD-L1 expression, was associated with improved overall survival (51). High PD-L1 expression on melanoma were found predominantly in regions of abundant inflammation or TIL infiltrates, even in sanctuaries like brain metastases (52), but it failed to predict responses to ICB in MMel. To inventors' knowledge, this is the first comprehensive analysis of the predictive role of PD-L1 expression on peripheral blood T cells in melanoma. This expression might reflect the chronic exposure to type 1-type 2 IFNs in the TME in recirculating TILs ((47) and not shown), as already reported in tumor cells themselves (53).

Inventors believe that given its biological relevance (24-31, 54-56) and co-expression of a variety of inhibitory receptors on CD95⁺CD4⁺ T cells (such as PD1, and HLA-DR, FIG. 11), higher levels (cut-off values >70%) of the CD95⁺CD4 T cells biomarker detected in MMel (compared with HV), is to be considered as a valuable biomarker.

The combination of immune checkpoint inhibitors ipilimumab and nivolumab has been FDA-approved for first-line treatment of unresectable MMel. This approval followed the results of CheckMate 067(20)-069(57), trials where the combination of ipilimumab and nivolumab outperformed each single agent alone in terms of response rates, PFS and OS. Additionally, recently published data in non-small cell lung cancer patients have shown promising results for the combination of anti-PD-L1 and anti-CTLA-4 mAbs in a phase 1b clinical trial (58). Hence, such combinations may be integrated into the ever-changing melanoma treatment algorithm, and will be most likely extended to other malignancies sensitive to PD-1 blockade. However, drug-related adverse events of grade 3 or 4 have been reported in 54% of patients receiving ipilimumab/nivolumab combination therapy, as compared with 24% of patients receiving ipilimumab monotherapy (59, 60). Such immune-related adverse events are generally reversible with immunosuppressive medications. Given the efficacy and relative safety of nivolumab alone, finding a predictor of response to such potentially toxic combinations is an urgent unmet clinical need. Here, inventors reveal a biomarker of response to ipilimumab+nivolumab: the presence of detectable levels of CD137 on blood CD8⁺ T cells, which appears to be significantly associated with a lack of relapse in resected high-risk, treatment-naïve stage III MMel. This novel biomarker is based on the following data: (i) circulating T lymphocytes expressing CD137 could be found in the blood of patients with no evidence of disease at 13 months who received the combination in an adjuvant setting (and not in those where nivolumab was administered alone); (ii) the finding from the ex vivo mLN assay that CD137 is upregulated in CD4⁺ and CD8⁺ TILs in lesions qualifying as “responding” to ex vivo stimulation with the combination of anti-PD-1+anti-CTLA-4 mAbs (and not to anti-PD-1 mAb or to other combinatorial regimens). It is therefore conceivable that this combinatorial stimulation leads to the engagement of the CD137/CD137L co-stimulatory pathway, required for T cell fitness and recirculation in the blood of responders (47). However, this pathway did not appear responsible for immediate tumor rejection mediated by the combination regimen in mouse models, although the addition of an agonistic CD137 mAb to the combination therapy further delayed tumor outgrowth in a therapeutic MCA-induced sarcoma model (MJS, unpublished data). This data confirms a previous study performed in mouse ovarian carcinomas, where agonistic anti-CD137 mAb augmented the impact of anti-PD-1+anti-CTLA-4 mAb therapy (61).

These novel predictive immunometrics add to the long list of putative biomarkers potentially relevant for ICB therapies. Inventors previous experience suggested that high LDH levels, CXCL11 and sCD25 concentrations in the serum negatively predict time to progression in ipilimumab-treated stage IV MMel (32, 62-64), whereas CLA expressing CD8⁺ TEM represent a pharmacodynamic signature of sensitivity to CTLA-4 blockade (47). HLA subtype (33), genetic polymorphisms (34), and absolute lymphocyte counts (35) have not been validated as immunotherapy biomarkers, a number of alternative parameters such as high baseline levels of Foxp3 and IDO expression (34), increased TILs and Th1 cells at baseline (36), MDSC numbers (62, 37, 65), T cell ICOS expression as pharmacodynamic markers (38), and (more recently) high mutational load and neoantigen landscape (39, 66), have yet to be prospectively studied as biomarkers for the efficacy of immunotherapy for melanoma. A number of biomarkers of response to anti-PD-1/PD-L1 mAbs have been considered promising for future prospective validation. For example, selective CD8⁺ T cell tumor infiltration (often correlated with PD-L1 expression) and their distribution at tumor invasive margins preceding PD-1 blockade appear to predict ORR in stage IV melanoma (40-42). Similarly, the immunohistochemical determination of PD-L1 expression (although lacking a standardized methodology and subject to variable expression depending on timing and biopsy sites) may guide the choice between PD-1 blockade versus CTLA-4+PD-1 co-blockade (41-43). A high mutational load is also associated with clinical responses to the PD-1 regimen (39, 44). Moreover, high relative eosinophil count, and lymphocyte count, low LDH and absence of metastasis other than soft-tissue or lung at baseline are associated with a favorable OS in patients treated with pembrolizumab (67). The proposed blood biomarkers herein identified (PD1 expression on CD4⁺ T cells, PD-L1 expression on CD4⁺ and CD8⁺ T cells, or the CD8⁺ T cell/Treg ratio in blood) are also useful to predict the efficacy of PD-1 blockade. Inventors' findings indicate that prospective ICB adjuvant trials in stage III-IV MMel can be personalized based on i) ex vivo mLN assays or ii) blood biomarkers capable of predicting such response.

### Materials and Methods

### Experimental Design

In a cohort of stage III MMel patients, inventors previously reported the immune parameters that were significantly associated with outcome (example 1). They established an ex vivo assay based on the reactivity of immune cells from 37 dissociated metastatic lymph nodes to mAbs and cytokines. They arbitrarily defined “responding” lesions, those exhibiting a more than 1.5-fold change over two different controls (medium and IgG) in two independent biological readouts (out of 40 readouts measured, 35 were retained with a threshold of 95% of detected values). This stratification into responders (R) versus non-responders (NR) enabled them to define in a retrospective manner which parameters expressed by peripheral or infiltrating T cells were essential for this response. Furthermore, they demonstrated the validity of the method by analyzing the predictive value of some parameters on retrospective clinical cohorts including 190 unresectable stage III-IV MMel patients.

### Patients and Cohorts

Study Approval.

Institutional review board approvals were granted by the University of Tubingen, the University of California, the University Hospital of Copenhagen, the University Hospital of Zürich, the University Hospital of Siena, the Sharett Institute of Oncology, the Aarhus University Hospital, the Centre Hospitalier de Nantes, and the Providence Cancer Center (for the ipilimumab-treated cohorts), the Laura & Isaac Perlmutter Cancer Center (for ipilimumab+nivolumab) and Gustave Roussy/Kremlin Bicêtre and Centre Hospitalier Lyon-Sud for the prospective cohort and retrospective cohorts (FIG. 11C) which were previously described (68, 69). The human study protocols were in accordance with the Declaration of Helsinki principles, and all patients provided informed consent before enrollment in the study.

Prospective Cohorts of 37 Patients.

This cohort and its clinical parameters have been previously described (example 1).

Retrospective Cohorts of 190 Ipilimumab-Treated Patients.

Patients enrolled in this study were from the University of Tubingen, University of Siena, University of California, University Hospital of Copenhagen, University Hospital of Zurich, Sharett Institute of Oncology, Aarhus University Hospital, Centre Hospitalier de Nantes, and Providence Cancer Center (Table 7). In all cohorts, blood samples were collected before injections of ipilimumab from patients participating in evaluations of ipilimumab as adjuvant therapy. Markers were assessed on PBMCs with the exception of the JE cohort (assessed on whole blood) after thawing. Patients' characteristics can be found in Table 7.

Retrospective Study on the Adjuvant Phase II Trial Testing Nivolumab+Ipilimumab Versus Nivolumab-Treated Patients.

Information regarding this clinical trial can be found in reference (70).

Peripheral blood mononuclear cells (PBMC) and TILs preparations have already been described (example 1).

Ex-Vivo mLN Assays.

See example 1.

Inventors arbitrarily defined “biological responses”, as those exhibiting a >1.5 fold increase over the values obtained with two negative controls (medium and Ig control mAb) in at least two independent biological readouts, except for CD4⁺FoxP3⁺ Treg for which a response was defined as a >1.5 fold decrease compared with the baseline levels in responders compared to non-responders.

Flow Cytometric Analyses.

For membrane labeling, PBMC and TILs were stained with fluorochrome-coupled mAbs (detailed in Table 4), incubated for 20 min at 4° C. and washed. Cell samples were acquired on a Cyan ADP 9-color (Beckman Coulter), BD FACS Canto II flow-cytometers or on an 18-color BD LSRII (BD Biosciences) with single-stained antibody-capturing beads used for compensation (Compbeads, BD Biosciences or UltraComp eBeads, eBiosciences). Data were analyzed with Flowjo software v7.6.5 or v10 (Tree Star, Ashland, Oreg., USA).

Cytokine and Chemokine Measurements.

Supernatants from cultured cells were monitored using the human Th1/Th2/Th9/Th17/Th22 13-plex RTU FlowCytomix Kit (eBiosciences), and human chemokine 6-plex kit FlowCytomix (eBiosciences) according to the manufacturer's instructions and acquired on a Cyan ADP 9-color flow cytometer (Beckman Coulter). Analyses were performed by FlowCytomix Pro 3.0 Software (eBiosciences). Some measurements were performed by ELISA with IFNγ (BioLegend), IL-9 (BioLegend), TNFα (BD Biosciences), CCL2 (BD Biosciences), CCL3 (R&D Systems), CCL4 (R&D Systems), CCL5 (R&D Systems) and CXCL10 (BD Biosciences) kits in accordance with the manufacturer's recommendations.

Statistics.

Data analyses were performed with the statistical environment R (see Worldwide Website: R-project.org/). Graphical representations were performed either with the statistical environment R or Prism 5 (GraphPad, San Diego, Calif., USA). In all, 124 (blood) and 128 (tumor) parameters were considered in analyses and reporting. Individual data points representing individual patient measurements are systematically graphed within box and whiskers plots calculated from the corresponding distribution. Comparisons between clinical groups were performed by Wilcoxon rank sum test for parameters expressed in percentage and ratios after log transformation. Logistic regressions (univariate and multivariate) have been used to evaluate the association of covariates on binary endpoint (i.e. tumor response). Overall survival (OS) was defined as the time from the date of sampling to death or the last follow-up, whichever occurred first. Progression-free survival (PFS) was defined as the time from the date of sampling to death, disease progression or the last follow-up, whichever occurred first. For both survival endpoints (OS and PFS), survival curves were estimated using the Kaplan-Meier method by dichotomizing biomarkers through their median value or a chosen cut-off. Cox models have been used to perform univariate and multivariate analysis. Graphical visualization of the effect of continuous biomarkers has been performed by modeling them through splines with 2 degrees of freedom. All the logistic and Cox models evaluated the biomarkers based on a continuous scale, were stratified on the centers, and adjusted for LDH, gender, age, tumor stage, CT, IT and PKI as indicated in Table 6 and Tables 8-10. Unless stated, p-values are two-sided and 95% confidence intervals for the statistic of interest are reported. Effectiveness of the biomarker was determined from empirical ROC curve and corresponding AUC are reported and graphed together with Wilcoxon rank sum test p-values in order to determine the best immunometrics in the ex-vivo mLN assay.

TABLE 9
Association between CD95 and PD-L1 (continuous scale) and the
ipilimumab responses (PD vs SD + PR + CR)

| Model | CD95.CD4 | CD95.CD8 | PDL1.CD4 | PDL1.CD8 |
|---|---|---|---|---|
| Univariate | 0.978 | 0.992 | 0.996 | 0.977 |
| | [0.959; 0.997] | [0.973; 1.011] | [0.959; 1.034] | [0.947; 1.006] |
| | P = 0.023 | P = 0.40 | P = 0.84 | P = 0.13 |
| Stratify on center | 0.979 | 1.007 | 0.995 | 0.972 |
| | [0.958; 1.001] | [0.984; 1.032] | [0.941; 1.053] | [0.924; 1.020] |
| | P = 0.060 | P = 0.56 | P = 0.87 | P = 0.25 |
| Stratify on center | 0.980 | 1.000 | 0.963 | 0.937 |
| Adjust for LDH, | [0.955; 1.005] | [0.968; 1.031] | [0.893; 1.033] | [0.869; 1.001] |
| gender, age, | P = 0.12 | P = 0.98 | P = 0.31 | P = 0.068 |
| tumor stage, CT, | | | | |
| IT and PKI* | | | | |

Odds ratios and 95% confidence intervals. Final model designated with an *.

TABLE 10
Association between CD95/CD4 (>70 vs. _70)
and overall survival

| | Hazard Ratio | 95% CI | p-value |
|---|---|---|---|
| Expression of CD95/CD4 | 1.96 | [1.10-3.48] | 0.022 |
| (>70 vs. ≤70) | | | |
| LDH status | 2.61 | [1.51-4.52] | 0.001 |
| Age | 0.979 | [0.963-0.996] | 0.018 |
| Gender | 0.90 | [0.58-1.42] | 0.66 |
| Tumor stage | 0.61 | [0.11-3.29] | 0.56 |
| Previous CT | 1.36 | [0.78-2.39] | 0.28 |
| Previous IT | 0.76 | [0.46-1.24] | 0.27 |
| Previous PKI | 1.33 | [0.60-2.95] | 0.48 |

CI: confidence interval. Adjusting covariates are in italic

### REFERENCES

1. L. Lacroix, et al, Genomes in the clinic: the Gustave Roussy Cancer Center experience, Curr. Opin. Genet. Dev. 24, 99-106 (2014).
2. J. Menis, et al, New clinical research strategies in thoracic oncology: clinical trial design, adaptive, basket and umbrella trials, new end-points and new evaluations of response, Eur. Respir. Rev. Off. J. Eur. Respir. Soc. 23, 367-378 (2014).
3. E. Tartour, L. Zitvogel, Lung cancer: potential targets for immunotherapy, Lancet Respir. Med. 1, 551-563 (2013).
4. V. Sathyanarayanan, S. S. Neelapu, Cancer immunotherapy: Strategies for personalization and combinatorial approaches, Mol. Oncol. (2015), doi:10.1016/j.molonc.2015.10.009.
5. P. W. Kantoff, et al, IMPACT Study Investigators, Sipuleucel-T immunotherapy for castration-resistant prostate cancer, N. Engl. J. Med. 363, 411-422 (2010).
6. S. A. Rosenberg, Raising the bar: the curative potential of human cancer immunotherapy, Sci. Transl. Med. 4, 127ps8 (2012).
7. C. H. June, et al, Adoptive cellular therapy: a race to the finish line, Sci. Transl. Med. 7,280ps7 (2015).
8. D. Przepiorka, et al, FDA Approval: Blinatumomab, Clin. Cancer Res. Off. J. Am. Assoc. Cancer Res. 21, 4035-4039 (2015).
9. J. D. Wolchok, et al, Four-year survival rates for patients with metastatic melanoma who received ipilimumab in phase II clinical trials, Ann. Oncol. Off. J. Eur. Soc. Med. Oncol. ESMO 24, 2174-2180 (2013).
10. S. L. Topalian, et al, Immune checkpoint blockade: a common denominator approach to cancer therapy, Cancer Cell 27, 450-461 (2015).
11. D. Killock, Skin cancer: T-VEC oncolytic viral therapy shows promise in melanoma, Nat. Rev. Clin. Oncol. 12, 438 (2015).
12. S. L. Topalian, et al, Immunotherapy: The path to win the war on cancer?, Cell 161, 185-186 (2015).
13. J. R. Brahmer, et al, Safety and activity of anti-PD-L1 antibody in patients with advanced cancer, N. Engl. J. Med. 366, 2455-2465 (2012).
14. E. B. Garon, et al, KEYNOTE-001 Investigators, Pembrolizumab for the treatment of non-small-cell lung cancer, N. Engl. J. Med. 372, 2018-2028 (2015).
15. S. Kleffel, et al, Melanoma Cell-Intrinsic PD-1 Receptor Functions Promote Tumor Growth, Cell 162, 1242-1256 (2015).
16. S. Ladoire, et al, Combined evaluation of LC3B puncta and HMGB1 expression predicts residual risk of relapse after adjuvant immunotherapy in breast cancer, Autophagy 11, 1878-1890 (2015).
17. R. B. Holmgaard, et al, Tumor-Expressed IDO Recruits and Activates MDSCs in a Treg-Dependent Manner, Cell Rep. 13, 412-424 (2015).
18. R. Kridel, et al, The Prognostic Impact of CD163-Positive Macrophages in Follicular Lymphoma: A Study from the BC Cancer Agency and the Lymphoma Study Association, Clin. Cancer Res. Off. I Am. Assoc. Cancer Res. 21, 3428-3435 (2015).
19. C. Denkert, et al, Tumor-infiltrating lymphocytes and response to neoadjuvant immunotherapy with or without carboplatin in human epidermal growth factor receptor 2-positive and triple-negative primary breast cancers, J. Clin. Oncol. Off. J. Am. Soc. Clin. Oncol. 33, 983-991 (2015).
20. J. Larkin, et al, Combined Nivolumab and Ipilimumab or Monotherapy in Untreated Melanoma, N. Engl. J. Med. 373, 23-34 (2015).
21. B. Weide, et al, Immunologic correlates in the course of treatment with immunomodulating antibodies, Semin. Oncol. 42, 448-458 (2015).
22. P. A. Ascierto, et al, Clinical experiences with anti-CD137 and anti-PD-1 therapeutic antibodies, Semin. Oncol. 37, 508-516 (2010).
23. T. I. Hewala, et al, The clinical significance of serum soluble Fas and p53 protein in breast cancer patients: comparison with serum CA 15-3, Pathol. Oncol. Res. POR 18, 841-848 (2012).
24. S.-M. Sheen-Chen, et al, Circulating soluble Fas in patients with breast cancer, World J. Surg. 27, 10-13 (2003).
25. A. I. El-Sarha, et al, Serum sFas and tumor tissue FasL negatively correlated with survival in Egyptian patients suffering from breast ductal carcinoma, Pathol. Oncol. Res. POR 15, 241-250 (2009).
26. P. J. Perik, et al, Circulating apoptotic proteins are increased in long-term disease-free breast cancer survivors, Acta Oncol. Stockh. Swed. 45, 175-183 (2006).
27. T. Ueno, et al, Circulating soluble Fas concentration in breast cancer patients, Clin. Cancer Res. Off. J. Am. Assoc. Cancer Res. 5, 3529-3533 (1999).
28. P. Redondo, et al, Fas and Fas ligand: expression and soluble circulating levels in cutaneous malignant melanoma, Br. J. Dermatol. 147, 80-86 (2002).
29. S. Ugurel, et al, Increased soluble CD95 (sFas/CD95) serum level correlates with poor prognosis in melanoma patients, Clin. Cancer Res. Off. J. Am. Assoc. Cancer Res. 7, 1282-1286 (2001).
30. L. Rivoltini, et al, Human melanoma-reactive CD4+ and CD8+ CTL clones resist

Fas ligand-induced apoptosis and use Fas/Fas ligand-independent mechanisms for tumor killing, J. Immunol. Baltim. Md. 1950 161, 1220-1230 (1998).
31. M. Malleter, et al, CD95L cell surface cleavage triggers a prometastatic signaling pathway in triple-negative breast cancer, Cancer Res. 73, 6711-6721 (2013).
32. Hannani D et al, Anticancer immunotherapy by CTLA4 blockade: obligatory contribution of IL-2 receptors and negative prognostic impact of soluble CD25, Cell Res.
33. J. D. Wolchok, et al, Ipilimumab efficacy and safety in patients with advanced melanoma: a retrospective analysis of HLA subtype from four trials, Cancer Immun. 10, 9 (2010).
34. O. Hamid, et al, A prospective phase II trial exploring the association between tumor microenvironment biomarkers and clinical activity of ipilimumab in advanced melanoma, J. Transl. Med. 9, 204 (2011).
35. G. Y. Ku, et al, Single-institution experience with ipilimumab in advanced melanoma patients in the compassionate use setting: lymphocyte count after 2 doses correlates with survival, Cancer 116, 1767-1775 (2010).
36. R.-R. Ji, et al, An immune-active tumor microenvironment favors clinical response to ipilimumab, Cancer Immunol. Immunother. CII 61, 1019-1031 (2012).
37. S. Kitano, et al, Computational algorithm-driven evaluation of monocytic myeloid-derived suppressor cell frequency for prediction of clinical outcomes, Cancer Immunol. Res. 2, 812-821 (2014).
38. D. Ng Tang, et al, Increased frequency of ICOS+ CD4 T cells as a pharmacodynamic biomarker for anti-CTLA-4 therapy, Cancer Immunol. Res. 1, 229-234 (2013).
39. A. Snyder, et al, Genetic basis for clinical response to CTLA-4 blockade in melanoma, N. Engl. J. Med. 371, 2189-2199 (2014).
40. P. C. Tumeh, et al, PD-1 blockade induces responses by inhibiting adaptive immune resistance, Nature 515, 568-571 (2014).
41. J. M. Taube, et al, Colocalization of inflammatory response with B7-hl expression in human melanocytic lesions supports an adaptive resistance mechanism of immune escape, Sci. Transl. Med. 4, 127ra37 (2012).
42. J. R. Brahmer, et al, Phase I study of single-agent anti-programmed death-1 (MDX-1106) in refractory solid tumors: safety, clinical activity, pharmacodynamics, and immunologic correlates, J. Clin. Oncol. Off. J. Am. Soc. Clin. Oncol. 28, 3167-3175 (2010).
43. M. Sznol, L. Chen, Antagonist antibodies to PD-1 and B7-H1 (PD-L1) in the treatment of advanced human cancer, Clin. Cancer Res. Off. J. Am. Assoc. Cancer Res. 19, 1021-1034 (2013).
44. M. S. Lawrence, et al, Mutational heterogeneity in cancer and the search for new cancer-associated genes, Nature 499, 214-218 (2013).
45. Eggermont, A. M. M., Spatz, A. & Robert, C. Cutaneous melanoma. Lancet Lond. Engl. 383, 816-827 (2014).
46. Jacquelot, N. et al. Immunophenotyping of stage III melanoma reveals parameters associated with patient prognosis. J. Invest. Dermatol. (2016). doi:10.1016/j.jid.2015.12.042
47. Jacquelot, N. et al. Chemokine receptor patterns in lymphocytes mirror metastatic spreading in melanoma. J. Clin. Invest. (2016). doi:10.1172/JCI80071
48. Eggermont, A. M. M. et al. Adjuvant ipilimumab versus placebo after complete resection of high-risk stage III melanoma (EORTC 18071): a randomised, double-blind, phase 3 trial. Lancet Oncol. 16, 522-530 (2015).
49. Eggermont, A. M. M. et al. Prolonged Survival in Stage III Melanoma with Ipilimumab Adjuvant Therapy. N. Engl. J. Med. 375, 1845-1855 (2016).
50. Rusakiewicz, S. et al. NKp30 isoforms and NKp30 ligands are predictive biomarkers of response to imatinib mesylate in metastatic GIST patients. OncoImmunology 00-00 (2016). doi:10.1080/2162402X.2015.1137418
51. Obeid, J. M. et al. PD-L1, PD-L2 and PD-1 expression in metastatic melanoma: Correlation with tumor-infiltrating immune cells and clinical outcome. Oncoimmunology 5, e1235107 (2016).
52. Berghoff, A. S. et al. Tumour-infiltrating lymphocytes and expression of programmed death ligand 1 (PD-L1) in melanoma brain metastases. Histopathology 66, 289-299 (2015).
53. Benci, J. L. et al. Tumor Interferon Signaling Regulates a Multigenic Resistance Program to Immune Checkpoint Blockade. Cell 167, 1540-1554.e12 (2016).
54. Klebanoff, C. A. et al. Memory T cell-driven differentiation of naive cells impairs adoptive immunotherapy. J. Clin. Invest. 126, 318-334 (2016).
55. Gajate, C. & Mollinedo, F. Lipid raft-mediated Fas/CD95 apoptotic signaling in leukemic cells and normal leukocytes and therapeutic implications. J. Leukoc. Biol. 98, 739-759 (2015).
56. Motz, G. T. et al. Tumor endothelium FasL establishes a selective immune barrier promoting tolerance in tumors. Nat. Med. 20, 607-615 (2014).
57. Hodi, F. S. et al. Combined nivolumab and ipilimumab versus ipilimumab alone in patients with advanced melanoma: 2-year overall survival outcomes in a multicentre, randomised, controlled, phase 2 trial. Lancet Oncol. 17, 1558-1568 (2016).
58. Antonia, S. et al. Safety and antitumour activity of durvalumab plus tremelimumab in non-small cell lung cancer: a multicentre, phase 1b study. Lancet Oncol. (2016). doi:10.1016/S1470-2045(15)00544-6
59. Postow, M. A. et al. Nivolumab and ipilimumab versus ipilimumab in untreated melanoma. N. Engl. J. Med. 372, 2006-2017 (2015).
60. Weber, J. et al. Phase I/II Study of Metastatic Melanoma Patients Treated with Nivolumab Who Had Progressed after Ipilimumab. Cancer Immunol. Res. 4, 345-353 (2016).
61. Wei, H. et al. Combinatorial PD-1 Blockade and CD137 Activation Has Therapeutic Efficacy in Murine Cancer Models and Synergizes with Cisplatin. PLoS ONE 8, e84927 (2013).
62. Martens, A. et al. Baseline peripheral blood biomarkers associated with clinical outcome of advanced melanoma patients treated with ipilimumab. Clin. Cancer Res. Off. J. Am. Assoc. Cancer Res. (2016). doi:10.1158/1078-0432.CCR-15-2412
63. Delyon, J. et al. Experience in daily practice with ipilimumab for the treatment of patients with metastatic melanoma: an early increase in lymphocyte and eosinophil counts is associated with improved survival. Ann. Oncol. Off. J. Eur. Soc. Med. Oncol. ESMO 24, 1697-1703 (2013).
64. Koguchi, Y. et al. Serum Immunoregulatory Proteins as Predictors of Overall Survival of Metastatic Melanoma Patients Treated with Ipilimumab. Cancer Res. 75, 5084-5092 (2015).
65. Sade-Feldman, M. et al. Clinical significance of circulating CD33+CD11b+HLA-DR− myeloid cells in Stage-IV melanoma patients treated with ipilimumab. Clin. Cancer Res. Off. J. Am. Assoc. Cancer Res. (2016). doi: 10.1158/1078-0432.CCR-15-3104
66. Riaz, N. et al. Recurrent SERPINB3 and SERPINB4 mutations in patients who respond to anti-CTLA4 immunotherapy. Nat. Genet. 48, 1327-1329 (2016).
67. Weide, B. et al. Baseline biomarkers for outcome of melanoma patients treated with pembrolizumab. Clin. Cancer Res. Off. J. Am. Assoc. Cancer Res. (2016). doi:10.1158/1078-0432.CCR-16-0127
68. Kruit, W. H. J. et al. Phase ½ study of subcutaneous and intradermal immunization with a recombinant MAGE-3 protein in patients with detectable metastatic melanoma. Int. J. Cancer J. Int. Cancer 117, 596-604 (2005).
69. Escudier, B. et al. Vaccination of metastatic melanoma patients with autologous dendritic cell (DC) derived-exosomes: results of the first phase I clinical trial. J. Transl. Med. 3, 10 (2005).
70. Nikhil I. Khushalani et al. Adjuvant nivolumab (NIVO) plus ipilimumab (IPI) for resected high-risk stages IIIC/IV melanoma (MEL). ASCO Meet. Abstr. (2016).

1-21. (canceled)

22. An in vitro method of predicting, assessing or monitoring the sensitivity of a subject having a cancer to an immunotherapy selected from anti-PD-1 monoclonal antibody, anti-PD-L1 monoclonal antibody, anti-CTLA-4 monoclonal antibody, anti-CD137 monoclonal antibody, anti-CD137L monoclonal antibody, anti-TIM3 monoclonal antibody, IFNα2a (ROF), IL-2, a combination of anti-PD-1 and anti-CTLA-4 monoclonal antibodies, a combination of anti-PD-1 monoclonal antibody and ROF, a combination of anti-CTLA-4 monoclonal antibody and ROF, and a combination of anti-PD-1 and anti-TIM3 monoclonal antibodies, which method comprises a step a) of determining, in a biological sample from said subject which is a blood sample or a sample comprising tumor cells, the presence, absence or expression level of at least one biomarker selected from PD-1⁺CD4⁺ T cells, CD8+ T cells and CD25⁺CD127⁻CD4⁺ T cells, CD95⁺CD4⁺ T cells, CD95⁺CD8⁺ T cells, PD-L1⁺CD4⁺ T cells, PD-L1⁺CD8⁺ T cells, CLA⁺CD8⁺TEM cells, CD137L⁺CD4⁺ T cells, CD137L⁺CD8⁺ T cells, CD137⁺CD4⁺ T cells and CD137⁺CD8⁺ T cells, and, when the expression level is determined, a step b) of comparing said at least one expression level to a reference expression level or to a reference expression ratio, thereby predicting, assessing or monitoring whether the subject having a cancer is responsive or resistant to the immunotherapy.

23. The method according to claim 22, wherein the step of determining the presence, absence or expression level of the at least one biomarker in a biological sample of the subject is performed before any immunotherapeutic treatment step, and optionally after at least partial tumor resection in the subject.

24. The method according to claim 22, wherein the cancer is selected from melanoma, lung, renal cancer, head and neck cancer, bladder cancer.

25. The method according to claim 22, wherein the immunotherapy is anti-PD-1 monoclonal antibody and the method comprises a step a) of determining, in a blood sample of the subject, the expression level of PD-1⁺CD4⁺ T cells, and a step b) of comparing said PD-1⁺CD4⁺ T cells level to a PD-1⁺CD4⁺ T cells reference expression level, an expression level of PD-1⁺CD4⁺ T cells above the PD-1⁺CD4⁺ T cells reference expression level being indicative of sensitivity of the subject to the immunotherapy and an expression level of PD-1⁺CD4⁺ T cells below the PD-1⁺CD4⁺ T cells reference expression level being indicative of resistance of the subject to the immunotherapy, and/or a step a′) of determining, in a blood sample of the subject, the expression levels of CD8⁺ T cells and of CD25⁺CD127⁻CD4⁺ T cells, and a step b′) of determining the ratio of CD8⁺ T cells/CD25⁺CD127⁻CD4⁺ T cells, a ratio above the reference expression ratio being indicative of sensitivity of the subject to the immunotherapy and a ratio below the reference expression ratio being indicative of resistance of the subject to the immunotherapy,

26. The method according to claim 25, wherein the PD-1⁺CD4⁺ T cells reference expression level is the percentage of CD4⁺ T cells expressing PD-1, an expression level of PD-1⁺CD4⁺ T cells in the subject corresponding to a percentage of CD4⁺ T cells expressing PD-1 above 21.06% being indicative of sensitivity of the subject to the immunotherapy, and an expression level of PD-1⁺CD4⁺ T cells in the subject corresponding to a percentage of CD4⁺ T cells expressing PD-1 below 7.45% being indicative of resistance of the subject to the immunotherapy.

27. The method according to claim 25, wherein a ratio above 5.4 is indicative of sensitivity of the subject to the immunotherapy and a ratio below 2.8 is indicative of resistance of the subject to the immunotherapy.

28. The method according to claim 22, wherein the immunotherapy is anti-CTLA-4 monoclonal antibody and the method comprises a step a) of determining, in a biological sample of the subject, the expression level of CD95⁺CD4⁺ T cells, of determining in a blood sample of the subject the expression level of CD95⁺CD8⁺ T cells, of determining in a blood sample of the subject the expression level of PD-L1⁺CD4⁺ T cells, and/or of determining in a blood sample of the subject the expression level of PD-L1⁺CD8⁺ T cells, and a step b) of comparing said levels to their respective reference expression levels, an expression level above the reference expression level being indicative of resistance of the subject to the immunotherapy, and an expression level below the reference expression level being indicative of sensitivity of the subject to the immunotherapy.

29. The method according to claim 28, wherein the CD95⁺CD4⁺ T cells reference expression level is the percentage of CD4⁺ T cells expressing CD95, an expression level of CD95⁺CD4⁺ T cells in the subject corresponding to a percentage of CD4⁺ T cells expressing CD95 above 70.80% in a sample comprising tumor cells or above 68.1% in a blood sample being indicative of resistance of the subject to the immunotherapy, and an expression level of CD95⁺CD4⁺ T cells in the subject corresponding to a percentage of CD4⁺ T cells expressing CD95 below 43.79% in a sample comprising tumor cells or below 48.5% in a blood sample being indicative of sensitivity of the subject to the immunotherapy.

30. The method according to claim 28, wherein the CD95⁺CD8 T cells reference expression level is the percentage of CD8⁺ T cells expressing CD95, an expression level of CD95⁺CD8⁺ T cells in the subject corresponding to a percentage of CD8⁺ T cells expressing CD95 above 74.48% being indicative of resistance of the subject to the immunotherapy, and an expression level of CD95⁺CD8⁺ T cells in the subject corresponding to a percentage of CD8⁺ T cells expressing CD95 below 44.13% being indicative of sensitivity of the subject to the immunotherapy.

31. The method according to claim 28, wherein the PD-L1⁺CD4⁺ T cells reference expression level is the percentage of CD4⁺ T cells expressing PD-L1, an expression level of PD-L1⁺CD4⁺ T cells in the subject corresponding to a percentage of CD4⁺ T cells expressing PD-L1 above 27.76% being indicative of resistance of the subject to the immunotherapy, and an expression level of PD-L1⁺CD4⁺ T cells in the subject corresponding to a percentage of CD4⁺ T cells expressing PD-L1 below 6.66% being indicative of sensitivity of the subject to the immunotherapy, and the PD-L1⁺CD8⁺ T cells reference expression level is the percentage of CD8⁺ T cells expressing PD-L1, an expression level of PD-L1⁺CD8⁺ T cells in the subject corresponding to a percentage of CD8⁺ T cells expressing PD-L1 above 21.45% being indicative of resistance of the subject to the immunotherapy, and an expression level of PD-L1⁺CD8⁺ T cells in the subject corresponding to a percentage of CD8⁺ T cells expressing PD-L1 below 2.53% being indicative of sensitivity of the subject to the immunotherapy.

32. The method according to claim 28, wherein the method comprises a step of determining the expression levels of CD95⁺CD4⁺ T cells and PD-L1⁺CD8⁺ T cells in a blood sample of the subject, an expression level of CD95⁺CD4⁺ T cells in the subject corresponding to a percentage of CD4⁺ T cells expressing CD95 above 70% together with an expression level of PD-L1⁺CD8⁺ T cells in the subject corresponding to a percentage of CD8⁺ T cells expressing PD-L1 above 11% being indicative of resistance of the subject to the immunotherapy.

33. The method according to claim 22, wherein the immunotherapy is anti-CTLA-4 monoclonal antibody and the method comprises a step a) of determining, in a blood sample of the subject three weeks after the first injection of the anti-CTLA4 monoclonal antibody, the percentage and/or absolute number of CLA⁺CD8⁺ TEM cells, and a step b) of comparing said percentage and/or absolute number with a reference percentage and/or absolute number of CLA⁺CD8⁺ TEM cells, a percentage and/or absolute number above the reference percentage and/or absolute number being indicative of sensitivity of the subject to the immunotherapy, and a percentage and/or absolute number below the reference percentage and/or absolute number being indicative of resistance of the subject to the immunotherapy.

34. The method according to claim 33, wherein a percentage of CLA⁺CD8⁺ TEM cells above 26.9 and/or absolute number above 33 cells per mm³ is indicative of sensitivity of the subject to the immunotherapy and a percentage of CLA⁺CD8⁺ TEM cells below 6 and/or absolute number below 14 cells per mm³ is indicative of resistance of the subject to the immunotherapy.

35. The method according to claim 22, wherein the immunotherapy is a combination of anti-PD-1 and anti-CTLA-4 monoclonal antibodies, and the method comprises a step a) of determining, in a blood sample of the subject, the expression level of CD137L⁻CD4⁺ T cells, CD137L⁺CD8⁺ T cells, CD137⁺CD4⁺ T cells and/or CD137⁺CD8⁺ T cells, and a step b) of comparing said level(s) to their respective reference expression level(s), an expression level of CD137L⁺CD4⁺ T cells and/or CD137L⁺CD8⁺ T cells above the reference expression level being indicative of resistance of the subject to the immunotherapy, and an expression level of CD137L⁺CD4⁺ T cells and/or CD137L⁺CD8⁺ T cells below the reference expression level being indicative of sensitivity of the subject to the immunotherapy, and an expression level of CD137⁺CD4⁺ T cells and/or CD137⁺CD8⁺ T cells below the reference expression level being indicative of resistance of the subject to the immunotherapy, and an expression level of CD137⁺CD4⁺ T cells and/or CD137⁺CD8⁺ T cells above the reference expression level being indicative of sensitivity of the subject to the immunotherapy.

36. The method according to claim 35, wherein the CD137L⁺CD4⁺ T cells reference expression level is the percentage of CD4⁺ T cells expressing CD137L, an expression level of CD137L⁺CD4⁺ T cells in the subject corresponding to a percentage of CD4⁺ T cells expressing CD137L above 25.19% being indicative of resistance of the subject to the immunotherapy, and an expression level of CD137L⁺CD4⁺ T cells in the subject corresponding to a percentage of CD4⁺ T cells expressing CD137L below 9.01% being indicative of sensitivity of the subject to the immunotherapy, and the CD137L⁺CD8⁺ T cells reference expression level is the percentage of CD8⁺ T cells expressing CD137L, an expression level of CD137L⁺CD8⁺ T cells in the subject corresponding to a percentage of CD8⁺ T cells expressing CD137L above 16.65% being indicative of resistance of the subject to the immunotherapy, and an expression level of CD137L⁺CD8⁺ T cells in the subject corresponding to a percentage of CD8⁺ T cells expressing CD137L below 7.86% being indicative of sensitivity of the subject to the immunotherapy.

37. The method according to claim 22 wherein the biological sample comprising tumor cells is selected from a tumor biopsy, a whole tumor piece, a tumor bed sample, and a metastatic lymph node cells sample.

38. The method according to claim 22, wherein the anti-CTLA-4 monoclonal antibody is selected from ipilimumab and tremelimumab.

39. The method according to claim 22, wherein the anti-PD-1 monoclonal antibody is selected from nivolumab and pembrolizumab.

40. A method of selecting an appropriate chemotherapeutic treatment for a subject having a cancer, which method comprises a step of predicting or assessing the sensitivity of a subject having a cancer to an immunotherapy using a method according to claim 22.

41. A kit for predicting, assessing or monitoring the sensitivity of a subject having a tumor to a cancer therapy, wherein the kit comprises, as detection means, at least two antibodies selected from the group consisting of an antibody specific to PD-1⁺CD4⁺ T cells, CD8+ T cells and CD25⁺CD127⁻CD4⁺ T cells, CD95⁺CD4⁺ T cells, CD95⁺CD8⁺ T cells, PD-L1⁺CD4⁺ T cells, PD-L1⁺CD8⁺ T cells, CLA⁺CD8⁺ TEM, CD137L⁺CD4⁺ T cells, CD137L⁺CD8⁺ T cells, CD137⁺CD4⁺ T cells and CD137⁺CD8⁺ T cells, and, optionally, a leaflet providing the corresponding reference expression levels.

42. An assay (“mLN assay”) for determining whether a patient is sensitive or resistant to a cancer therapy, wherein the assay comprises:
a first step wherein suspensions of metastatic lymph nodes samples are incubated ex vivo in duplicate wells, each well of each set of the duplicate being in contact with medium, with a control antibody, or with a test immunotherapeutic antibody defining a cancer therapy, said antibody being selected from an anti-PD-1 monoclonal antibody, an anti-PD-L1 monoclonal antibody, an anti-CTLA-4 monoclonal antibody, an anti-CD137 monoclonal antibody, an anti-CD137L monoclonal antibody, an anti-TIM3 monoclonal antibody, an IFNα2a (ROF), an IL-2, a combination of anti-PD-1 and anti-CTLA-4 monoclonal antibodies, a combination of anti-PD-1 monoclonal antibody and ROF, a combination of anti-CTLA-4 monoclonal antibody and ROF, or a combination of anti-PD-1 and anti-TIM3 monoclonal antibodies, the first set of wells being incubated for 18h-24h, and the second set of wells being incubated for 4 to 5 days,
a second step of measuring T cells, NK cells and/or Treg cells parameters, said parameters consisting in cell biomarker(s) expression, cytokine cell release, interferon γ cell release, chemokine cell release and/or interleukin cell release in the first set of wells, and Ki67 cell expression and Treg cell proportion in the second set of wells, and
a third step of comparing measures obtained in each well with the corresponding measure obtained from the medium and control wells, a 1.5 fold variation of at least two parameters indicating that the patient is sensitive to the cancer therapy.
